(19) United States
(12) Patent Application Publication
(10) Pub. No.: US 2024/0041830 A1
(43) Pub. Date: Feb. 8, 2024

(54) CIRCULATING BIOMARKERS OF RESPONSE TO PD-1/PD-L1 BLOCKADE AND GSK-3 INHIBITION

(71) Applicant: Brown University, Providence, RI (US)

(72) Inventors: Wafik S. EL-DEIRY, Barrington, RI (US); Kelsey E. HUNTINGTON, Providence, RI (US); Benedito A. CARNEIRO, East Greenwich, RI (US)

(21) Appl. No.: 18/365,964

(22) Filed: Aug. 4, 2023

Related U.S. Application Data
(60) Provisional application No. 63/370,483, filed on Aug. 4, 2022.

(51) Int. Cl. A61K 31/407; G01N 33/574; A61P 35/00; A61K 39/395; C07K 16/28

(52) U.S. Cl. CPC A61K 31/407; G01N 33/57419; A61P 35/00; A61K 39/3955; C07K 16/2827; A61K 2039/505

(57) ABSTRACT

A method of treating cancer, where the biomarkers are predictive of response to PD-1/PD-L1 blockade and GSK-3 inhibition, and which can be used in the method of treatment.

### CROSS-REFERENCE TO RELATED APPLICATIONS

This patent application claims priority under 35 U.S.C. § 119(e) to the provisional patent applications U.S. Ser. No. 63/370,483, filed Aug. 4, 2022.

### TECHNICAL FIELD OF THE INVENTION

This invention generally relates to generally relates to heterocyclic compounds containing both one or more hetero rings having oxygen atoms as the only ring hetero atoms, and one or more rings having nitrogen as the only ring hetero atom containing three or more hetero rings, and specifically to GSK-3 inhibitors belong to a class of maleimide derivatives containing a maleimide group.

### BACKGROUND OF THE INVENTION

Globally, colorectal cancer (CRC) ranks third in terms of incidence and second in terms of mortality. Patients with microsatellite stable (MSS) colorectal cancer represent about 85% of the total patient population. The percentage of patients with MSS colorectal cancer increases to ˜96% in Stage IV disease. Gupta, Sinha, & Paul, Curr. Probl. Cancer, 42, 548-559 (2018). Treatment options include surgery, chemotherapy, radiation therapy, targeted therapy, and immunotherapy. Immune checkpoint blockade (ICB) has now entered into clinical care for colorectal cancer with the recent U.S. Food & Drug Administration approvals of checkpoint inhibitors nivolumab and pembrolizumab for microsatellite instability-high (MSI-H) colorectal cancer cases after chemotherapy. Borelli et al., Cancers, 14, 4974 (2022). Immune checkpoint blockade therapy shows impressive efficacy in microsatellite instability positive (MSI+) colorectal cancer but has shown disappointing results in MSS colorectal cancer. Thus, there remains a substantial unmet need in the ˜85% of patients with MSS colorectal cancer in whom immune checkpoint blockade is less effective. Gupta, Sinha, & Paul, Curr. Probl. Cancer, 42, 548-559 (2018).

Recent scientific literature characterizes the immunomodulatory roles of glycogen synthase kinase 3 (GSK-3) in the context of anti-tumor immunity. GSK-3 is a serine/threonine kinase with key roles in many biological processes, including tumor progression. The GSK3β/beta-catenin axis activates vascular endothelial growth factor (VEGF) signaling in endothelial cells to promote angiogenesis. VEGF is known to be immunosuppressive. VEGF dampens the immune cell response by promoting recruitment of tumor associated macrophages (TAMs). Inhibition of GSK-3 using a small-molecule elraglusib has shown promising preclinical antitumor activity in several tumor types.

The response rate to immune checkpoint blockade in MSS colorectal cancer patients is very limited, especially as the tumor stage advances, thus, there is a clear need in the biomedical art for improved treatment strategies for this patient population.

### SUMMARY OF THE INVENTION

In a first embodiment, the invention provides a method of treatment, where the biomarkers are predictive. Among the relevant biomarkers provided in this specification that are predictive of response to PD-1/PD-L1 blockade and GSK-3 inhibition and which can be used in the method of treatment are the following:

Elevated pre-pharmacokinetic (PK) plasma concentrations of IL-12, Fas Ligand, IL-8, M-CSF, IL-2, IL-15, CCL7, and CCLII correlated with improved progression-free survival (PFS) in days.

Decreased plasma concentrations of CXCLII and VEGF correlated with improved progression-free survival.

At the 8-hour post-PK timepoint, increased IL-8 concentrations correlated with improved progression-free survival.

At the 24-hour post-PK timepoint, increased levels of IL-12, IL-1 beta, IL-21, IL-8, IFN-alpha, IFN-gamma, M-CSF, CCL4, Fas Ligand, IL-2, IL-10, CCLII, IL-15, IL-4, and Granzyme B were correlated with improved progression-free survival.

Reduced CXCLII plasma concentrations correlated with worsened progression-free survival.

Elevated IL-8, CCLII, IFN-alpha, Fas Ligand, TRAIL R2, and IL-1 beta were correlated with improved overall survival.

Decreased levels of CXCLII and TNF-alpha were correlated with improved overall survival.

At the 8-hour post-PK timepoint CCL22 and IL-8 levels were positively correlated with overall survival.

At the 24-hour post-PK timepoint IFN-alpha, Fas Ligand, TRAIL R2, and CCLII levels were positively correlated with overall survival.

Complete and partial responders, regardless of treatment group, are more likely to have lower serum concentrations of BAFF, CCL7, CCL12, VEGF, VEGFR2, and CCL21 compared to non-responders.

Complete and partial responders have higher serum concentrations of CCL4, TWEAK, GM-CSF, CCL22, and IL-12p70 compared to non-responders.

In a second embodiment, the PD-1/PD-L1 blockade part of the method of treatment is anti-PD-L1. The inventors found that anti-PD-L1 in combination with elraglusib was more efficacious than the combination of anti-PD-1 and elraglusib.

In a third embodiment, key biomarkers of response were evaluated at baseline in treatment-naïve patients and monitored longitudinally and assist in the evaluation of tumor response to treatment and guide therapeutic decisions. These biomarkers are response markers to immune checkpoint blockade and GSK-3 inhibition.

Evaluating the combination of immune checkpoint blockade with small molecules in oncology is one of the ways the biomedical art can improve the efficacy of immune checkpoint blockade in microsatellite stable colorectal cancer patients. The inventors focused on the small-molecule inhibitor of GSK-3 elraglusib (9-ING-41). The inventors characterized several immunomodulatory mechanisms that provide a clinical rationale for the combination of GSK-3 inhibitors such as elraglusib in combination with immune checkpoint blockade. The inventors evaluated the immunomodulatory impact of small molecule GSK-3 inhibitor elraglusib (9-ING-41) in combination with immune checkpoint blockade in a syngeneic murine model as well as in patients with refractory solid tumors of multiple tissue origins enrolled in a Phase 1 clinical trial investigating the GSK-3 inhibitor (NCT03678883). The inventors found that murine and human plasma or serum concentrations of several circulating factors were predictive of response to PD-1/PD-L1 blockade and GSK-3 inhibition.

The inventors showed that elraglusib treatment increased tumor cell PD-L1 expression, downregulated angiogenic and immunosuppressive signaling pathways, and increased anti-tumor immune responses in vitro and in vivo.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 is a set of drawings and charts that shows that elraglusib enhances pyroptosis and sensitizes tumor cells to increase immune-mediated cytotoxicity in a co-culture model. Co-cultures were treated with drug concentrations as indicated. A 1:1 effector to target (E:T) ratio was used with a 24-hour co-culture duration. EthD-1 was used to visualize dead cells; 10× magnification; scale bar indicates 100 μm. FIG. 1A is a set of six representative SW480 and TALL-104 T cell co-culture assay images at the 24-hour timepoint; twenty-four tumor cell pre-treatment with 5 μM elraglusib, followed by 24-hour co-culture. FIG. 1B is a chart showing the quantification of the co-culture experiment using the percentage of dead cells out of total cells (N=3). FIG. 1C is a chart showing the quantification normalized by cell death observed with drug treatment alone (N=3). FIG. 1D is a set of six representative SW480 and donor-derived CD8+ T cell co-culture assay images at the 24-hour timepoint; 24-hour tumor cell pre-treatment with 5 μM elraglusib, followed by a 24-hour co-culture. FIG. 1 is a chart showing the quantification of the co-culture experiment using the percentage of dead cells out of total cells (N=3). FIG. 1F is a chart showing the quantification normalized by cell death observed with drug treatment alone (N=3). FIG. 1G is a chart showing the number of HCT 116 GFP+ cells were quantified after forty-eight hours of culture with DMSO, 5 μM elraglusib, or 5000 TALL-104 cells (N=3). FIG. 1H is a chart showing the number of HT-29 GFP+ cells was quantified after forty-eight hours of culture with DMSO, 5 μM elraglusib, or 5000 TALL-104 cells (N=3). FIG. 1I is a chart showing the number of HCT 116 GFP+ cells was quantified after forty-eight hours of culture with DMSO, 5 μM elraglusib, or 5000 NK-92 cells (N=3). FIG. 1J is a chart showing the number of HT-29 GFP+ cells was quantified after forty-eight hours of culture with DMSO, 5 μM elraglusib, or 5000 NK-92 cells (N=3). FIG. 1K is a set of eight representative 40× images of HCT-116 GFP+ or HT-29 GFP+ colorectal cancer and TALL-104 T cell co-cultures. Black arrows indicate pyroptotic events. FIG. 1L and FIG. 1M are representative images of western blot analysis of (L) HCT-116 colorectal cancer cells and (M) HT-29 colorectal cancer cells for expression of indicated proteins after treatment with indicated cytokines or drugs. FIG. 1N and FIG. 1O are charts showing the quantification of IFN-γ secretion (pg/mL) post-DMSO or elraglusib treatment for twenty-four hours in (N) TALL-104 cells and (O) NK-92 cells (N=3). Error bars represent the mean +/31 standard deviation. Statistical test: one-way ANOVA with Tukey's test for multiple comparisons. p-value legend: * p<0.05, ** p<0.01, *** p<0.001, **** p<0.0001.

FIG. 2 is a set of images showing elraglusib treatment induces PD-L1 expression and suppresses survival pathways in tumor cells. FIG. 2A is the set of representative images of Western blot analysis of HCT-116 colorectal cancer cells and HT-29 colorectal cancer cells for expression of indicated proteins after increasing durations of elraglusib treatment (0-72 hours). FIG. 2A is the set of representative images of western blot analysis of Mcl-1 expression in HCT-116 CRC cells after increasing durations of elraglusib treatment. Colorectal cancer cells were treated with 1 μM elraglusib for twenty-four hours and treated versus untreated samples were compared in triplicate. The inventors later visualized the microarray analysis results using volcano plots for HCT-116, HT-29, and KM12C colorectal cancer cell lines (N=3). The results were calculated using a fold-change cutoff of >1.5, <−1.5, and a p-value of <0.05. The number of genes up-regulated or down-regulated in each of the three cell lines within several signaling pathways of interest (N=3). The inventors preventors prepared a Venn Diagram to compare the 3124 genes that were differentially expressed post-treatment with elraglusib in the three colon cancer (HCT-116, HT-29, KM12C) cell lines (N=3). Tumor cells (HCT-116, HT-29) were treated with 1 μM elraglusib for forty-eight hours and cell culture supernatant was analyzed with the Luminex 200. Tumor cells (HCT-116, HT-29) were treated with 5 μM elraglusib for forty-eight hours and cell culture supernatant was analyzed with the Luminex 200.

FIG. 3 is a set of images showing elraglusib treatment increases effector molecule secretion and induces an energetic shift in cytotoxic immune cells. FIG. 3A is the set of representative images of Western blot analysis of TALL-104. FIG. 3B is the set of representative images of Western blot analysis of HT-29 cytotoxic immune cells for expression of indicated proteins after increasing durations of elraglusib treatment (0-72 hours). FIG. 3C is a diagram of the proposed model for non-canonical NF-κB pathway activation: increased NIK expression indicates non-canonical NF-κB pathway activation which enhances the expression of chemokines and cytokines (CCL11, TNF-α, GM-CSF) and subsequently leads to increased recruitment and proliferation of cytotoxic immune cells (CD8+T, CD4+T, NK cells). Immune cells were treated with 1 μM elraglusib for twenty-four hours and treated versus untreated samples were compared in triplicate. The inventors visualized microarray analysis using volcano plots for NK-92 and TALL-104 immune cell lines (N=3). The inventors prepared a Venn diagram to compare the 124 genes that were differentially expressed post-treatment with elraglusib in the two immune (TALL-104, NK-92) cell lines (N=3). The inventors performed 10× single-cell sequencing analysis of immune cells treated with elraglusib. TALL-104 and NK-92 cells were treated with 1 μM elraglusib for twenty-four hours and aggregate data were visualized using a t-SNE plot. Immune cells show differential expression of mitochondria-encoded genes (MD and ribosomal genes (RB) after elraglusib treatment. Immune cells (TALL-104, NK-92) were treated with 1 μM elraglusib for forty-eight hours and cell culture supernatant was analyzed with the Luminex 200.

FIG. 4 is a set of charts showing that elraglusib enhances immune-cell tumor-infiltration to prolong survival in combination with anti-PD-L1 therapy in a syngeneic murine model of MSS colon carcinoma. FIG. 4A is a diagram of an overview of the syngeneic murine colon carcinoma BALB/c murine model using MSS cell line CT-26. FIG. 4B is a Kaplan-Meier estimator curves for all treatment groups as indicated. Statistical significance was determined using a Log-rank (Mantel-Cox) test. FIG. 4C is an overview of cell lineage markers used for flow cytometric immunophenotyping analysis. Two weeks after treatment initiation, immune cell subpopulations were analyzed in the spleen and tumor. FIG. 4D is a chart for splenic T cells. FIG. 4E is a chart for tumor-infiltrating T cells. FIG. 4F is a chart for splenic NK cells, FIG. 4G is a chart for tumor-infiltrating NK cells were compared between responders (R, N=3) and non-responders (NR, N=18), regardless of treatment group. NK cell subsets based on the expression of

CD11b and CD27 were compared in the spleen and visualized via (FIG. 4H) bar graph and (FIG. 4I) pie chart. NK cell subsets based on the expression of CD11b and CD27 were also compared in the tumor and visualized via (FIG. 4J) bar graph and (FIG. 4K) pie chart. T cell ratios were compared in the (FIG. 4L) spleen and (FIG. 4M) tumor. Statistical significance was determined using two-tailed unpaired T tests. p-value legend: * p<0.05, ** p<0.01, *** p<0.001, **** p<0.0001. ns: non-significant.

FIG. 5 is a set of charts showing that responders have a more immunostimulatory tumor microenvironment as compared to non-responders. Immunohistochemistry analysis of tumors two weeks post-treatment initiation or tumors from long-term mice. Non-responders (NR, N=18) and responders (R, N=3) were compared. 20× images, scale bar represents 100 μm. (A,B) CD3, (C,D) Granzyme B, (E,F) Ki67, (G,H) PD-L1, and (I,J) cleaved-caspase 3 (CC3) were compared at the two weeks after treatment initiation timepoint, and the long-term timepoint, respectively. Statistical significance was determined using two-tailed unpaired T tests (N=6). Serum from long-term mice sacrificed was analyzed via cytokine profiling for (K) CCL21, (L) VEGFR2, (M) CCL7, (N) CCL12, (0) BAFF, (P) VEGF, (Q) IL-1 β, (R) IL-6, (S) CCL22, (T) GM-CSF, (U) CCL4, (V) TWEAK, and (W) CCL2. Responders (red, N=3) and non-responders (black, N=18) were compared. A Kruskal-Wallis test was used to calculate statistical significance, followed by a Benjamin-Hochberg correction for multiple comparisons. p values are shown for analytes that were significantly different between responders and non-responders and are ordered by significance. p-value legend: * p<** p<0.01, *** p<0.001. ns: no significance.

FIG. 6 is a table (TABLE 1) showing patient plasma concentrations of cytokines correlate with progression-free survival (PFS), overall survival (OS), and in vivo response to therapy results. Plasma samples from human patients with refractory solid tumors of multiple tissue origins enrolled in a Phase 1 clinical trial investigating a GSK-3 inhibitor elraglusib (NCT03678883) were analyzed using a Luminex 200 (N=19). TABLE 1 summarizes patient demographics. Baseline and 24-hour post-elraglusib plasma concentrations of cytokines, chemokines, and growth factors were plotted against PFS and overall survival. Simple linear regressions were used to calculate significance. p values less than 0.05 were reported as statistically significant. Cytokines grouped by function. Fold-change is shown where green indicates a negative (<0) fold-change compared to the baseline (pre-dose) value and red indicates a positive (>0) fold-change.

FIG. 7 shows the spatial profiling of patient tumor biopsies reveals a more immunostimulatory tumor microenvironment after elraglusib treatment. Patient samples were analyzed using NanoString GeoMx Digital Spatial Profiling (DSP) technology (N=12 biopsies). (A) Pie charts showing biopsy timepoint, primary tumor type, metastatic biopsy tissue type, and paired/unpaired biopsy sample information breakdowns. (B) A representative region of interest (ROI) showing PanCK+ and CD45+ masking. Green indicates CK, red indicates CD45, and blue indicates DAPI staining. (C) A Sankey diagram was used to visualize the study design where the width of a cord in the figure represents how many segments are in common between the two annotations they connect. The scale bar represents 50 segments. Blue cords represent CD45+segments and yellow cords represent panCK+ segments. (E) PanCK+ ROI CD39 expression plotted against time-on-study (TOS). Points are color-coded by time on study (TOS)/time on treatment with darker blue points indicating a shorter TOS or time on treatment. (F) CD45+ ROI CD163 expression plotted against TOS.

FIG. 8. Elraglusib increases immune-mediated cytotoxicity in a co-culture model with colorectal cancer cells. (A) Representative SW480 and TALL-104 T cell co-culture assay images at the 24-hour timepoint. 24-hour tumor cell pre-treatment with 5 μM elraglusib, followed by 24-hour co-culture. EthD-1 was used to visualize dead cells, 10× magnification, scale bar indicates 100 μm. (B) Quantification of coculture experiment using the percentage of dead cells out of total cells (N=3). (C) Quantification normalized by cell death observed with drug treatment alone (N=3). (D) Representative SW480 and donor-derived CD8+ T cell co-culture assay images at the 24-hour timepoint. (E) Quantification of co-culture experiment using the percentage of dead cells out of total cells (N=3). (F) Quantification normalized by cell death observed with drug treatment alone (N=3). A one-way ANOVA followed by a post-hoc Dunnett's multiple comparisons test was used to calculate statistical significance.

FIG. 9 shows that colorectal cancer cell lines selected represent diverse mutational backgrounds and exhibit varying elraglusib IC-50 values. Tumor cell lines (HCT-116, HT-29, SW480) and immune cell lines (NK92, TALL-104) were treated for 24-hour or 72-hour cell viability was assessed to determine IC so values (N=3). TABLE 2 of colorectal cancer cell lines included in the study and their diverse mutational profiles.

FIG. 10. Internal controls for tumor cell microarray analysis. (A) PCA mapping demonstrated clear mapping of HCT-116, HT-29, and KM12C cells (N=3). (B) Quality control was determined satisfactory for further analysis. (C) Hybridization controls. (D) Labeling controls. (E) Pos vs Neg Area Under the Curve (AUC). (F) Signal box plot.

FIG. 11. Internal controls for immune cell microarray analysis. (A) PCA mapping demonstrated clear mapping of NK-91 and TALL-104 cells (N=3). (B) Quality control was determined satisfactory for further analysis. (C) Hybridization controls. (D) Labeling controls. (E) Pos vs Neg Area Under the Curve (AUC). (F) Signal box plot.

FIG. 12. Syngeneic murine colon carcinoma BALB/c murine model with MSS cell line CT-26 Kaplan Meier curves and mouse body weights grouped by treatment. Individual Kaplan Meier curves for isotype control (N=12) compared to (A) elraglusib (N=12), (B) anti-PD-1 (N=12), (C) anti-PD-L1 (N=12), (D) elraglusib+anti-PD-1 (N=12), and (E) elraglusib+anti-PD-L1 (N=12). (F) Bar graph indicating the percentage of responders (R) and non-responders (NR) per treatment group. Individual body weight plots for (G) Isotype control (N=12), (H) elraglusib (N=12), (I) anti-PD-1 (N=12), (J) anti-PD-L1 (N=12), (K) elraglusib+anti-PD-1 (N=12), and (L) elraglusib+anti-PD-L1 (N=12). P-value legend: * p<0.05, ** p<0.01.

### DETAILED DESCRIPTION OF THE INVENTION

### Industrial Applicability

The biomarkers identified in this disclosure are predictive of response to therapy. The biomarkers can be used by clinicians to determine which patients would be most likely to benefit from these types of therapy and to determine how the patient is responding to current therapy. These circulating biomarkers could be used as a non-invasive tool by clinicians to improve treatment strategies.

The inventors identified several key biomarkers of response that can be evaluated at baseline in treatment-naive patients and monitored longitudinally and assist in the evaluation of tumor response to treatment and guide therapeutic decisions. These biomarkers are response markers to immune checkpoint blockade and GSK-3 inhibition that could provide significant benefit as a tool used by clinicians and expand the therapeutic benefit of these interventions to a broader patient population diagnosed with malignancies.

These biomarkers could also guide the therapeutic development of GSK-3 inhibitors and identify patients more likely to benefit from these inhibitors when used alone or in combination with other anti-cancer therapies.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, the technical and scientific terms have the same meaning as commonly understood by a person having ordinary skill in the biomedical art. A term's meaning provided in this specification shall prevail if any apparent discrepancy arises between the meaning of a definition provided in this specification and the term's use in the biomedical art.

A or an means at least one or one or more unless the context indicates otherwise.

About has the plain meaning of approximately. The term about encompasses the measurement errors inherently associated with the relevant testing. When used with percentages, about means ±1%. About or approximately when referring to a value or parameter means to be within a range of normal tolerance in the art, e.g., within two standard deviations of the mean. A description referring to about X includes the description of X.

Adult T-cell leukemia/lymphoma (ATLL) has the biomedical art-recognized art meaning. Adult T-cell leukemia/lymphoma (ATLL) is a rare and aggressive mature T cell neoplasm associated with human T-cell lymphotropic virus (HTLV-1) infection. Expansion of these T-cells results from the expression of the viral oncoprotein Tax-1, which in turn activates transcription factors, e.g., cAMP-dependent transcription factor, nuclear factor kappa-B (NF-κB), inhibits apoptosis (e.g., repression of p53), disrupts cell cycle control, and interferes with genetic stability (DNA polymerase β, proliferating cell nuclear antigen, and the mitotic spindle-assembly checkpoint protein MAD1). See Proietti, Carneiro-Proietti, Catalan-Soares, & Murphy, Oncogene, 24,6058-6068 (2005).

Cancer therapy has the biomedical art-recognized meaning of anticancer treatment to cure or prolong the life of a mammal with cancer, especially a human with cancer. Among the cancer therapies known in the medical art include the following: Some therapies treat tumors with mutated p53. Some chemotherapies involve administering 5-fluorouracil (5-FU), irinotecan, etoposide, gemcitabine, oxaliplatin, carboplatin, paclitaxel, or a combination thereof to the subject who has cancer. Some radiotherapies involve administering radiation to the subject who has cancer. Some chemotherapies involve administering PARP inhibitors. Some therapies target. DNA repair-deficient cancers that may have defective repair of replicating DNA. Examples of DNA repair-deficient cancers include BRCA1-deficient cancers. Some chemotherapies involve administering immune checkpoint therapy, such as anti-PD-1, anti-PD-L1, or anti-CTLA4 antibodies. Some chemotherapies are targeted cancer therapies that involve administering anti-ATM, anti-ATR, anti-Chk1, anti-Chk2, anti-EGFR, anti-alk, anti-Her2, anti-NTRK, anti-BRAF, anti-KRAS antibodies to the subject who has cancer.

Circulating biomarkers of response to GSK-3 inhibition has the meaning described in this disclosure.

Contacting has the medical chemical art-recognized meaning of bringing together two compounds, molecules, or entities in an in vitro system or an in vivo system.

Effective Amount and Therapeutically Effective Amount include an amount sufficient to prevent or ameliorate a manifestation of the disease or medical condition, such as colorectal cancer. Many ways are known in the biomedical art to determine the effective amount for a given application. For example, pharmacological methods for dosage determination can be used in the therapeutic context. In the context of therapeutic or prophylactic applications, the amount of a composition administered to the subject depends on the type and severity of the disease and on the characteristics of the individual, such as general health, age, sex, body weight, and tolerance to drugs, and on the degree, severity, and type of disease. The skilled artisan can determine appropriate dosages depending on these and other factors. The compositions can also be administered in combination with one or more additional therapeutic compounds.

Elraglusib (9-ING-41) has the biomedical art-recognized art meaning of CAS No. 1034895-42-5; PubChem number ; CID 150974278; IUPAC Name: 3-(5-fluoro-1-benzofuran-3-yl)-4-(5-methyl-[1,3]dioxolo[4,5-f]indo1-7-yppyrrole-2,5-dione. Elraglusib is a maleimide-based ATP-competitive and selective inhibitor of glycogen synthase kinase-3 (GSK-3) that stimulates natural killer cells and T cells, reduces levels of vascular endothelial growth factor as well as other cytokine, chemokine, and growth factors in colorectal cancer cell supernatant, and stimulates killing of colon cancer cells. Elraglusib induces apoptosis and cell cycle arrest at prophase by targeting centrosomes and microtubule-bound GSK-3β. Elraglusib is commercially available from Selleck Chemicals, Houston, TX, USA (Catalog No. S9602). See Ugolkov et al., Anticancer Drugs, 29(8), 717-724 (September 2018). Elraglusib was licensed to Actuate Therapeutics.

Glycogen synthase kindase-3 (GSK-3) is a ubiquitously expressed protein kinase that exists in two isoforms (α, β), and is constitutively active. GSK-3 promotes the growth of some cancers, such as pancreatic cancers and colorectal cancers. See Ding et al., Clin. Cancer Res., 25, 6452-6462 (2019); Li J et al., Gastroenterology, 128, 1907-1918 (2005). GSK-3 is a positive regulator of NF-κB. GSK-3 promotes cancer cell survival and proliferation by facilitating chemoresistance. Medunjanin et al., Sci. Rep. 6, 38553 (2016). GSK-3 functions both as a proto-oncogene and as a tumor suppressor. Keith et al., International Journal of Cell Biology (2012). GSK-3 can phosphorylate β-catenin, triggering β-catenin destabilization and degradation, and maintaining low levels of β-catenin in the cytosol and nucleus. Inhibition of GSK-3 can lead to stabilization and activation of β-catenin, which could activate proliferative, tumorigenic pathways. Li J et al., Gastroenterology, 128, 1907-1918 (2005). GSK-3 is necessary for NF-κB signaling through modulation of NEMO phosphorylation. GSK-3 inhibition could suppress this inflammatory pathway. Medunjanin et al., Sci. Rep. 6, 38553 (2016). GSK-3 regulates the expression of checkpoint ligands in both immune and cancer cells. In CD8+ T cells, GSK-3 inhibition regulates PD-1 transcription and enhances T cell function. Taylor et al., Immunity, 44, 274-286 (2016).

Granzyme B has the biomedical art-recognized art meaning of the pro-apoptotic protease that is expressed by both cytotoxic lymphocytes and natural killer cells. Activation of CD8+ T cells induces the expression of granzyme B which is delivered, along with perforin, to target cells and induces apoptosis. Carneiro & EI-Deiry, Nature Rev. Clin. Oncol., 17, 395-417 (2020). elraglusib also increased the production of TRAIL by CD8+ T cells.

IFN-γ has the biomedical art-recognized art meaning of a key effector cytokine in immunity. IFN-γ upregulates major histocompatibility complex (MHC) molecules and the machinery involved in antigen processing and presentation. Castro et al., Front. Immunol., 9, 847 (2018). This upregulation of cell surface MHC class I by IFN-γ is essential for cytotoxic T cell activation, and thus, the host response to tumor cells. Maraskovsky, Chen, & Shortman, J. Immunol., 143, 1210-1214 (1989).

Immune Checkpoint Blockade (ICB) has the biomedical art-recognized art meaning. Immune checkpoint blockade (ICB) has now entered into clinical care for colorectal cancer with the recent U.S. Food & Drug Administration approvals of checkpoint inhibitors nivolumab and pembrolizumab for microsatellite instability-high (MSI-H) colorectal cancer cases after chemotherapy. Borelli et al., Cancers, 14, 4974 (2022).

Immunotherapy has the biomedical art-recognized art meaning.

p53 pathway restoration has the biomedical art-recognized meaning of a medical intervention effort to restore p53 activity as an anticancer therapeutic approach. See Martinez, Restoring p53 tumor suppressor activity as an anticancer therapeutic strategy. Future Oncol. 6(12), 1857-1862 (December 2010). The S-phase DNA damage response pathway is characterized by the increase in p-ATR(Thr1989). This increase ultimately leads to a delay in S-phase cells. This S-phase perturbation may contribute to cancer cell death.

p53, a tumor-suppressor, prevents cancer development via initiating cell-cycle arrest, cell death, repair, or anti-angiogenesis processes. Over 50% of human cancers harbor cancer-causing mutations in p53. p53 mutations not only abrogate its tumor-suppressor function, but also endow mutant p53 with a gain of function (GOF), creating a proto-oncogene that contributes to tumorigenesis, tumor progression, and chemotherapy or radiotherapy resistance. Targeting mutant p53 or restoring a wild-type p53 signaling pathway provides an attractive strategy for cancer therapy.

Pharmaceutically acceptable has the medical chemical art-recognized meaning that the compounds, materials, compositions, or dosage forms are within the scope of sound medical judgment and are suitable for contact with tissues of humans and other animals. The pharmaceutically acceptable compounds, materials, compositions, or dosage forms result in no persistent detrimental effect on the subject or the general health of the treated subject. Still, transient effects, such as minor irritation or a stinging sensation, are common with the administration of medicament and are consistent with the composition, formulation, or ingredient (e.g., excipient) in question. Guidance as to what is pharmaceutically acceptable is provided by comparable compounds, materials, compositions, or dosage forms listed in the US Pharmacopeia or another generally recognized pharmacopeia for use in animals, and more particularly in humans.

Pharmaceutically acceptable salts include, but are not limited to, salts of acidic or basic groups. Basic compounds can form a wide variety of salts with various inorganic and organic acids. Compounds that include an amino moiety may form pharmaceutically acceptable salts with various amino acids. Acidic compounds can form base salts with different pharmacologically acceptable cations. Salts include quaternary ammonium salts of the compounds described herein, where the compounds have one or more tertiary amine moiety.

Prevention or preventing has the medical chemical art-recognized meaning of reducing the risk of acquiring a disease, condition, or disorder.

Pyroptosis has the biomedical art-recognized art meaning of a lytic and pro-inflammatory type of programmed cell death that results in cell swelling and membrane perforation. Although the role of pyroptosis in cancer is controversial, it has been suggested that pyroptosis may contribute to anti-tumor immunity. See Lu, Guo, & Zhang, Cancers, 13, 3620 (2021).

Subject or patient has the biomedical art-recognized meaning of an organism, typically a mammal, e.g., a human. A subject can be susceptible to a disease, disorder, or condition, display one or more symptoms or characteristics of a disease, disorder or condition, or be someone with one or more features characteristic of susceptibility to or risk of a disease, disorder, or condition, but can also be a normal, healthy human.

Subject That Has A Cancer or Subject That Has A Tumor is a subject having objectively measurable cancer cells present in the subject's body. Included in this definition are malignant, actively proliferative cancers and potentially dormant tumors or micrometastases. Cancers that migrate from their original location and seed other vital organs can eventually lead to the death of the subject through the functional deterioration of the affected organs. Hemopoietic cancers, such as leukemia, can out-compete the regular hemopoietic compartments in a subject, thereby leading to a hemopoietic failure (in the form of anemia, thrombocytopenia, and neutropenia), ultimately causing death. A subject can have been previously diagnosed with or identified as suffering from or having a condition in need of treatment (e.g., a cancer) or one or more complications related to such a condition, and optionally, but need not have already undergone treatment for a condition or the one or more complications related to the condition. A subject can also not have been previously diagnosed as having a condition in need of treatment or one or more complications related to such a condition. For example, a subject can exhibit one or more risk factors for a condition, or one or more complications related to a condition or a subject who does not exhibit risk factors.

TRAIL has the biomedical art-recognized art meaning. Expression of TRAIL by immune cells induces apoptosis in tumor cells. Carneiro & EI-Deiry, Nature Rev. Clin. Oncol., 17, 395-417 (2020). In fact, GSK-3 inhibition has been shown to enhance both tumor necrosis factor-alpha- and TRAIL-induced apoptosis in pancreatic cell lines. Zhang et al., Cell Death Dis., 5, e1142 (2014).

Therapeutically effective amount has the medical chemical art-recognized meaning of the amount of active compound or pharmaceutical agent that elicits the biological or medicinal response that is being sought in a tissue, system, animal, individual or human by a researcher, veterinarian, medical doctor, or another clinician. The therapeutic effect is dependent upon the disorder being treated or the biological effect desired. As such, the therapeutic effect can be a decrease in the severity of symptoms associated with the disorder or inhibition (partial or complete) of progression of the disorder, or improved treatment, healing, prevention or elimination of a disorder, or side-effects. The amount needed to elicit the therapeutic response can be based on, for example, the age, health, size, and sex of the subject. Optimal amounts can also be determined based on monitoring of the subject's response to treatment.

Treat, treated, or treating has the medical chemical art-recognized meaning of both treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of the condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of the condition, disorder or disease. Treatment includes eliciting a clinically significant response, optionally without excessive levels of side effects. Treatment also includes prolonging survival as compared to expected survival if not receiving treatment.

Treat, Treatment, Treating, or Amelioration Of A Disease, Disorder Or Medical Condition means therapeutic treatments for a condition. The object is to reverse, alleviate, ameliorate, inhibit, slow down or stop the progression or severity of a symptom or condition. The term treating includes reducing or alleviating at least one adverse effect or symptom of a condition. Treatment is generally effective if one or more symptoms or clinical markers are reduced. Alternatively, treatment is effective if the progression of a condition is reduced or halted. That is, treatment includes not just the improvement of symptoms or markers but also a cessation or at least slowing of progress or worsening of symptoms that would be expected in the absence of treatment.

Unless otherwise defined, scientific and technical terms used with this application shall have the meanings commonly understood by persons having ordinary skill in the biomedical art. This invention is not limited to the method, protocols, reagents, etc., described herein and can vary.

This specification does not concern a process for cloning humans, methods for modifying the germ line genetic identity of humans, uses of human embryos for industrial or commercial purposes, or procedures for modifying the genetic identity of animals likely to cause them suffering with no substantial medical benefit to humans or animals resulting from such processes.

Guidance from Materials and Methods

Persons having ordinary skill in the biomedical art can use these materials and methods as guidance to predictable results when making and using the invention.

Cell culture maintenance. Human colorectal cancer cells SW480 (RRID: CVCL_0546), HCT-116 (RRID: CVCL_0291), HT-29 (RRID: CVCL_0320), and KM12C (RRID: CVCL_9547) were used in this study. SW480 cells were cultured in Dulbecco's Modified Eagle Medium (DMEM) supplemented with 10% FBS and 1% penicillin-streptomycin HCT-116 and HT-29 were cultured in McCoy's 5A (modified) Medium supplemented with 10% FBS and 1% penicillin-streptomycin. KM12C cells were cultured in Eagle's Minimal Essential Medium supplemented with 10% FBS and 1% penicillin-streptomycin. Human immune cells NK-92 (RRID: CVCL_2142), TALL-104 (RRID: CVCL_2771), and patient-derived CD8+ T cells were also used in this analysis. NK-92 cells were cultured in Alpha Minimum Essential medium supplemented with 2 mM L-glutamine, 1.5 g/L sodium bicarbonate, 0.2 mM inositol, 0.1 mM 2-mercaptoethanol, 0.02 mM folic acid, 12.5% horse serum, and 12.5% FBS. TALL-104 cells (CD2+; CD3+; CD7+; CD8+; CD56+; CD4−; CD16−) and patient-derived T cells (CD3+; CD8+) were cultured in RPMI-1640 containing 20% FBS, 100 U/mL penicillin, and 100 μg/mL streptomycin. Recombinant human IL-2 (Miltenyi cat #130-097744) with a final concentration of 100 units/mL was added to all immune cell culture media. All cell lines were incubated at 37° C. in a humidified atmosphere containing 5% CO₂. Cell lines were authenticated and tested to ensure the cultures were free of mycoplasma infection.

Measurement of cell viability. Cells were seeded at a density of 3×10³ cells per well in a 96-well plate (Greiner Bio-One, Monroe, NC, USA). Cell viability was assessed using the CellTiter Glo assay (Promega, Madison, WI, USA). Cells were mixed with 25 μL of CellTiter-Glo reagents in 100 μL of culture volume, and bioluminescence imaging was measured using the Xenogen IVIS imager (Caliper Life Sciences, Waltham, MA). The percent of cell viability was determined by normalizing the luminescence signal to control wells. Dose-response curves were generated, and the half-maximal inhibitory concentration (IC₅₀) was calculated using Graph-Pad Prism (RRID: SCR_002798) version 9.2.0. For IC₅₀ generation, concentrations were log-transformed, and data were then normalized to control, and a log (inhibitor) versus response (three parameters) test was used.

Pyroptosis assay. Recombinant Human TNF-α (Cat #300-01A, PeproTech, Rocky Hill, NJ, USA) and Recombinant Human IFN-γ (Cat #300-02, PeproTech, Rocky Hill, NJ, USA) were purchased for use in western blot analysis while rhTRAIL was generated in-house. See Kim et al., J. Biol. Chem., 279, 40044-40052 (2004).

TABLE 4

| Reagent | Source | Identifier | Dilution |
|---|---|---|---|
| Vinculin (E1E9V) XP ® | Cell Signaling | Cat# 13901 | 1:1000 |
| Rabbit mAb | | | |
| Anti-GSDMB antibody | Sigma-Aldrich | Cat# HPA052407 | 1:1000 |

Isolation of donor-derived CD8+ T cells. An Easy Step Human CD8+ T Cell Isolation Kit was used to isolate CD8+ T cells from a donor PBMC sample via negative selection (Cat #, 17913, Stem Cell Technologies, Vancouver, Canada).

Collection of cell culture supernatants used in cytokine measurements. Cells were plated at 3.5×10⁴ cells in a 48-well plate (Thermo Fisher Scientific, Waltham, MA, USA) in complete medium and incubated at 37° C. with 5% CO2. At twenty-four hours after plating, almost all the tumor cells were adherent to the bottom of the flask and the complete medium was replaced with the drug-containing medium. Subsequently, the culture supernatants were collected after forty-eight hours of incubation and were frozen at −80° C. until the measurement of cytokines was performed. On the day of analysis, samples were thawed and centrifuged to remove cellular debris.

Human cytokine profiling. Human cell line culture supernatants were analyzed using an R&D systems Human Premixed Multi-Analyte Kit (R&D Systems, Inc., Minneapolis, MN, USA) and a Luminex 200 (RRID: SCR_018025) Instrument (LX200-XPON-RUO, Luminex Corporation, Austin, TX, USA) according to the manufacturer's instructions. Sample levels of TNF-α, 4-1BB/TNFRSF9/CD137, IL-8/CXCL8, Ferritin, IFN-β, IL-10, CCL2/JE/MCP-1, VEGF, CXCL13/BLC/BCA-1, IFN-γ, CCL20/MIP-3 α, CCL3/MIP-1 α, CCL22/MDC, CCL4/MIP-1 β, Fas Ligand/TNFSF6, IL-17/1L-17A, IL-2, BAFF/BLyS/TNFSF13B, GM-CSF, CXCLS/ENA-78, TRANCE/TNFSF11/RANK L, CXCL9/MIG, G-CSF, IFN-γ R1/CD119, VEGFR3/Flt-4, C-Reactive Protein/CRP, CXCL11/I-TAC, IL-21, CXCL14/BRAK, IL-6, Fas/TNFRSF6/CD95, TRAIL R3/TNFRSF10C, IL-4, CCL5/RANTES, PD-L1/137-H1, CCL7/MCP-3/MARC, Chitinase 3-like 1, CXCL10/IP-10/CRG-2, IL-1 μ/IL-1F2, IL-7, Prolactin, CCL8/MCP-2, TRAIL R2/TNFRSF10B, M-CSF, IL-15, Granzyme B, IFN-α, TREM-1, IL-12/1L-23 p40, TRAIL/TNFSF10, CCL11/Eotaxin, and 1L-18/1L-1F4. Quantitative analysis with 6 standards and a minimum of 50 counts per bead region was used with the Luminex to generate analyte values reported as picograms/milliliter (pg/mL). Sample concentrations less than the lower limit of detection for each particular analyte were recoded as the lower limit value divided by 10. Sample concentrations above the upper limit of detection for a particular analyte were recoded as the upper limit of detection.

Murine cytokine profiling. Whole blood from mice was collected, allowed to clot, and serum was isolated using a serum separator tube (SST) according to manufacturer instructions. Murine serum samples were analyzed using an R&D systems Murine Premixed Multi-Analyte Kit (R&D Systems, Inc., Minneapolis, MN, USA) and a Luminex 200 (RRID: SCR_018025) Instrument (LX200-XPON-RUO, Luminex Corporation, Austin, TX, USA) according to the manufacturer's instructions. Sample levels of GM-CSF, IL-7, IL-12 p70, CCL2/JE/MCP-1, IL-1 β/IL-1F2, VEGF, IL-2, IL-4, VEGFR2/KDR/Flk-1, IL-6, IL-10, IL-13, IFN-γ, IL-3, IL-16, CXCL10/IP-10/CRG-2, CCL5/RANTES, CCL7/MCP-3/MARC, CCL12/MCP-5, Prolactin, M-CSF, CCL3/MIP-1 α, IL-1 α/IL-1F1, CCL20/MIP-3 α, CCL4/MIP-1 β, TWEAK/TNFSF12, CXCL12/SDF-1 α, BAFF/BLyS/TNFSF13B, Granzyme B, CCL21/6Ckine, CCL11/eotaxin, and CCL22/MDC. Sample values are reported in picograms per milliliter (pg/mL). Quantitative analysis with six standards and a minimum of fifty counts per bead region was used with the Luminex to generate analyte values reported as picograms/milliliter (pg/mL). Sample concentrations less than the lower limit of detection for each particular analyte were recoded as the lower limit value divided by 10. Sample concentrations above the upper limit of detection for a particular analyte were recoded as the upper limit of detection. Data analysis and visualization were generated using R (RRID: SCR_001905) software (R Development Core Team, 2020). When comparing responders and non-responders, a Kruskal-Wallis test was used to calculate statistical significance, followed by a Benjamini-Hochberg correction for multiple comparisons.

GFP+ cell line generation. A total of 50,000 HT-29 or HCT 116 cells were seeded in a 12-well tissue culture plate and allowed to adhere overnight. They were then transduced with lentivirus containing the plasmid pLenti_CMV_GFP_Hygro (pLenti CMV GFP Hygro (656-4) was a gift from Eric Campeau & Paul Kaufman (Addgene viral prep #17446-LV; RRID: Addgene_17446)) at a multiplicity of infection of ten with 8 μg/mL polybrene (hexadimethrine bromide (Cat #107689, Sigma Aldrich, St. Louis, MO, USA) for twenty-four hours, before washing with PBS and replacing with fresh medium. Campeau et al., PLoS ONE, 4, e6529 (2009). The cells were then sorted for GFP-positivity using a BD FACSAria™ III Cell Sorter (RRID: SCR_016695).

Multicolor immune cell co-culture experiments. A total of 10,000 HCT-116, SW480, or HT-29 colorectal cancer cells were plated per well in a clear-bottom, black-walled 48-well tissue culture plate and were allowed to adhere overnight. Cells were subsequently treated with DMSO, 5 μM or 10 μM elraglusib, or 10,000 TALL-104, donor-derived CD8+ T cells, or NK-92 cells (for an effector-to-tumor ratio of 1:1) for twenty-four hours. Immune cell monocultures were treated with DMSO, 5 μM or 10 μM elraglusib and cell viability was monitored to ensure that the concentrations of drug used were not cytotoxic to immune cells. Colorectal cancer cells were labeled using CellTracker™ Green CMFDA (5-chloromethyl fluorescein diacetate), immune cells (NK-92, TALL-104) were labeled using CellTracker™ Blue CMAC Dye (7-amino-4-chloromethylcoumarin). Ethidium homodimer-1 (EthD-1) was used as a marker of cell death (Invitrogen, Waltham, MA, USA). 10× images were captured using a Nikon Ti-U Inverted Fluorescence Microscope and NIS-Elements F Package imaging software 3.22.00 Build 710 (Nikon Instruments Inc., New York, NY, USA). The number of red/green color cells in random fields was determined using thresholding and particle analysis in the Fiji modification (RRID: SCR_002285) of ImageJ and expressed as a dead/live cell ratio. Normalization was carried out by subtracting the percentage of cell death due to drug or vehicle control (DMSO) from the percentage of dead cells observed in the co-culture of tumor and immune cells treated with the drug. At least one hundred cells were evaluated per sample, with three independent replicates. Statistical analysis was carried out using GraphPad Prism 9 (RRID: SCR_002798).

Single-color immune cell co-culture experiments. A total of 5000 HT-29 GFP+ or HCT 116 GFP+ cells were plated per well in a clear-bottom, black-walled 96-well tissue culture plate and were allowed to adhere overnight. Cells were subsequently treated with DMSO, 5 μM elraglusib, or 5000 TALL-104 or NK-92 cells (for an effector-to-tumor ratio of 1:1) for forty-eight hours. Nine images were taken per well at 10× magnification using a Molecular Devices ImageXpress® Confocal HT.ai High-Content Imaging System and quantified for the number of GFP+ objects using the MetaXpress (RRID: SCR_016654) software (Molecular Devices, San Jose, CA, USA). 40× images were also taken at twenty-four hours for representative images of cellular morphology changes. Statistical analysis was carried out using GraphPad Prism 9 (RRID: SCR_002798).

Generation of single-cell suspensions. Spleens were strained, filtered, and washed while tumors were collected, washed, and digested before lymphocytes were collected using a Percoll gradient (Cat #P1644-100ML, Sigma Aldrich, St. Louis, MO, USA).

Flow cytometry. Flow cytometry viability staining was conducted by suspending murine spleen and tumor single cell suspensions in Zombie Violet fixable viability kit (Cat #423114, BioLegend, San Diego, CA, USA) according to manufacturer instructions for thirty minutes at room temperature. Staining for membrane surface proteins was conducted using conjugated primary antibodies for one hour on ice, according to the manufacturer's instructions. Cells were fixed and permeabilized using the eBioscience™ Foxp3/Transcription Factor Staining Buffer Set according to manufacturer instructions (Cat #00-5523-00, Invitrogen, Waltham, MA, USA). Cells were resuspended in Flow Cytometry Staining Buffer (R&D Systems, Minneapolis, MN, USA) and analyzed using a BD Biosciences LSR II (RRID: SCR_002159) and FlowJo (RRID: SCR_008520) version 10.1 (FlowJo, Ashland, OR, USA).

TABLE 5

| Reagent | Source | Identifier | Concentration |
|---|---|---|---|
| Zombie Violet ™ Fixable | BioLegend | 423114 | 1:1000 |
| Viability Kit | | | |
| CD45 monoclonal antibody | eBioscience ™ | 83-0451-42 | 5 μL/test |
| (30-F11), eVolve 605 | | | |
| PE rat anti-mouse CD3 | BD | 555275 | 0.125 μg/test |
| molecular complex, clone | Biosciences | | |
| 17A2 (RUO) | | | |
| CD335 (NKp46) monoclonal | eBioscience ™ | 17-3351-82 | 0.125 μg/test |
| antibody (29A1.4), APC | | | |
| APC/Cy7 anti-mouse/human | BioLegend | 101226 | 0.125 μg/test |
| CD11b, clone: M1/70 | | | |
| Cd27 monoclonal antibody | eBioscience ™ | 11-0271-82 | 0.5 μg/test |
| (LG.7F9), FITC | | | |
| Klrg1 monoclonal antibody | eBioscience ™ | 25-5893-82 | 0.25 μg/test |
| (2F1), PE-Cyanine7 | | | |
| Anti-mouse CD45, | Invitrogen | 83-0451-42 | 0.5 μg/test |
| eBioscience, eVolve 605, | | | |
| clone: 30-F11 | | | |
| APC-Cy ™ 7 rat anti-mouse | BD | 560590 | 0.125 μg/test |
| CD3 molecular complex, | Biosciences | | |
| clone 17A2 | | | |
| CD4 monoclonal antibody | Invitrogen | 25-0042-82 | 0.25 μg/test |
| (RM4-5), PE-Cyanine7 | | | |
| PE rat anti-mouse CD8a, | BD | 553032 | 0.125 μg/test |
| clone 53-6.7 (RUO) | Biosciences | | |
| CD69 monoclonal antibody | eBioscience ™ | 11-0691-81 | 0.5 μg/test |
| (H1.2F3), FITC | | | |
| FOXP3 monoclonal | Manufacturer | 17-5773-82 | 1 μg/test |
| antibody (FJK-16s), APC | | | |

Natural killer cell immunophenotyping. The natural killer cell flow cytometry panel included the following directly conjugated primary antibodies: anti-mouse CD45, eBioscience eVolve 605 clone: 30-F11 (Ref #83-0451-42, Invitrogen), PE anti-mouse CD3 molecular complex (17A2) (mat. #: 555275, BD biosciences), anti-mouse NKp46 APC (Ref #17-3351-82), APC/Cy7 anti-mouse/human CD11 b clone: M1/70 (cat #101226, BioLegend), anti-Cd27 monoclonal antibody (LG.7F9) FITC (eBioscience™, Thermo Scientific, cat #11-0271-82), and (Klrg1 monoclonal antibody (2F1) PE-Cyanine7 (eBioscience, Thermo Scientific, cat #25-5893-82). Gating strategies are as follows:
NK cell: live/CD45/CD3−/NK1.1+
Mature NK cell: live/CD45/CD3−/NK1.1+/KRLG1+
Activated NK cell: live/CD45/CD3−/NK1.1+/CD11b+
NK cell subset 1: live/CD45/CD3−/NK1.1+/CD11b−CD27−
NK cell subset 2: live/CD45/CD3−/NK1.1+/CD11b−CD27+
NK cell subset 3: live/CD45/CD3−/NK1.1+/CD11b+CD27+
NK cell subset 4: live/CD45/CD3−/NK1.1+/CD11b+CD27−

T cell immunophenotyping. The T cell flow cytometry panel included the following directly conjugated primary antibodies: anti-mouse CD45 superbright 600 clone: 30-511 (ref #63-0451-82, eBioscience), anti-CD3 APC-Cy7 clone 17A2(BD Biosciences, cat #560590), eBioscience anti-mouse CD4 PE-Cy7 clone: RM4-5 (Ref #Invitrogen), PE anti-mouse CD8a (Ly-2)(53-6.7) (cat #553032, BD), anti-mouse CD69 FITC clone: H1.2F3 (Ref#11-0691-81, eBioscience), and Foxp3 (FJK-16s) APC (eBioscience). Gating strategies are as follows:
CD4+ T cell: live/CD45+/CD3+/CD4+/Foxp3−
CD8+T cell: live/CD45+/CD3+/CD8+
Treg: live/CD45+/CD3+/CD4+/Foxp3+
Activated CD8+ T cell: live/CD45+/CD3+/CD8+/CD69+

Western blot analysis. Cells were plated in a 6-well plate and incubated overnight before the spent media was replaced with drugged media. Drug treatment lasted for indicated durations. Protein was extracted using radioimmunoprecipitation (RIPA) assay buffer (Cat #R0278, Sigma-Aldrich, St. Louis, MO, USA) containing cOmplete™, Mini, EDTA-free Protease Inhibitor Cocktail (Cat #4693159001, Roche, Basel, Switzerland) from sub-confluent cells. Denaturing sample buffer was added, samples were boiled at 95° C. for ten minutes, and an equal amount of protein lysate was electrophoresed through NuPAGE™ 4 to 12%, bis-Tris, 1.5 mm, Mini Protein Gels (Invitrogen, Waltham, MA, USA) then transferred to PVDF membranes. The PVDF membrane was blocked with 5% non-fat milk (Sigma-Aldrich, St. Louis, MO, USA) in 1× TTBS. Primary antibodies were incubated with the transferred PVDF membrane in blocking buffer at 4° C. overnight. Secondary antibodies included Goat anti-Rabbit IgG (H+L) Secondary Antibody, HRP (Cat #31460, Invitrogen, Waltham, MA, USA), and Goat anti-Mouse IgG (H+L) Secondary Antibody, HRP (Cat #31430, Invitrogen, Waltham, MA, USA). The signal was detected using Pierce™ ECL Western Blotting Substrate (Cat #32106, Thermo Scientific, Waltham, MA, USA) and a Syngene Imaging System (RRID: SCR_015770).

TABLE 6

| Reagent | Source | Identifier | Concentration |
|---|---|---|---|
| PARP Antibody | Cell Signaling | Cat# 9542S | 1:1000 |
| Mcl-1 (D2W9E) Rabbit mAb | Cell Signaling | Cat# 94296S | 1:1000 |
| NF-κB p65 (L8F6) Mouse | Cell Signaling | Cat# 6956 | 1:1000 |
| mAb | | | |
| PD-L1 (E1L3N ®) XP ® | Cell Signaling | Cat# 13684 | 1:1000 |
| Rabbit mAb | | | |
| Bcl-2 (D55G8) Rabbit mAb | Cell Signaling | Cat# 4223S | 1:1000 |
| Survivin (71G4B7) Rabbit | Cell Signaling | Cat# 2808S | 1:1000 |
| mAb | | | |
| Mouse Anti-Ran | BD | Cat# 610341 | 1:5000 |
| | Biosciences | | |
| NIK Antibody | Cell Signaling | Cat# 4994 | 1:1000 |

In vivo studies. The experimental in vivo protocol (Protocol #19-01-003) was approved by the Institutional Animal Care and Use Committee of Brown University (Providence, RI, USA). Six-week to seven-week-old female BALB/c mice (RRID: IMSR_JAX:000651) were purchased from Taconic. A total of 50,000 cells were suspended in 50 μL ice-cold PBS and 50 μL Matrigel (Catalog #354234, Corning, New York, NY, USA), and 100 μL was injected subcutaneously into the rear flanks. Once tumor volume reached at least 100 mm³, mice were randomly assigned to one of seven groups (12 mice/group): Control (isotype), elraglusib, elraglusib+Isotype, anti-PD-1, anti-PD-L1, elraglusib+anti-PD-1, and elraglusib+anti-PD-L1. All treatments were delivered by IP injection using the following dosing schedule: Isotype (70 mg/kg, twice a week), elraglusib (70 mg/kg, twice a week), anti-PD-1 (10 mg/kg, twice a week), anti-PD-L1 (10 mg/kg, twice a week). Treatment dosing was determined based on previous studies of elraglusib and anti-PD-1/anti-PD-L1. Ugolkov et al., Anticancer Drugs, 29, 717-724 (2018); McClanahan et al., Blood, 126, 203-211 (2015); Bernardo et al., Oncoimmunology, 10, 1881268 (2021). The solvent for elraglusib was DMSO and the formulation buffer for elraglusib was made using 75% PEG400, 7% Tween 80, and 18% Ethanol. The treatment continued until mice developed signs of discomfort from excessive tumor growth. Mice were weighed once a week to monitor signs of drug toxicity. The length (L) and width (W) of the masses were measured three times per week with a digital caliper, and the tumor volume was calculated by applying the formula: 0.5LW2 Collection of whole blood and serum was performed by cardiac puncture and sent to Antech Diagnostics GLP (Morrisville, NC, USA) for blood cell count and chemistry tests, or in-house cytokine profiling. Tumors and organs were dissected and harvested for analysis by immunohistochemistry and flow cytometry.

Immunohistochemistry. Excised tissues were fixed with 10% neutral buffered formalin and paraffin-embedded. Five-micrometer tissue sections were cut with a microtome and mounted on glass microscope slides for staining. Hematoxylin and eosin staining was completed for all tumor specimens. Paraffin embedding and sectioning of slides were performed by the Brown University Molecular Pathology Core Facility. Slides were dewaxed in xylene and subsequently hydrated in ethanol at decreasing concentrations. Antigen retrieval was carried out by boiling the slides in 2.1 g citric acid (pH 6) for 10 minutes. Endogenous peroxidases were quenched by incubating the slides in 3% hydrogen peroxide for five minutes. After nuclear membrane permeabilization with Tris-buffered saline plus 0.1% Tween 20, slides were blocked with horse serum (Cat #MP-7401-15, Vector Laboratories, Burlingame, CA, USA), and incubated with primary antibodies overnight in a humidified chamber at 4° C. After washing with PBS, a secondary antibody (Cat #MP-7401-15 or MP-7402, Vector Laboratories, Burlingame, CA, USA) was added for 30 minutes, followed by diaminobenzidine application (Cat #NC9276270, Thermo Fisher Scientific, Waltham, MA, USA) according to the manufacturer's protocol. Samples were counterstained with hematoxylin, rinsed with distilled water, dehydrated in an increasing gradient of ethanol, cleared with xylene, and mounted with Cytoseal mounting medium (Thermo Fisher Scientific, catalog no. 8312-4). Images were recorded on a Zeiss Axioskop microscope (RRID: SCR_014587), using QCapture (RRID: SCR_014432). QuPath software (RRID: SCR_018257) was used to automatically count positive cells. For each immunohistochemistry marker, five 20× images per group were analyzed, and results were represented as the absolute number of positive cells per 20× field. The signal was quantified by converting randomly sampled 20× images into 16-bit images and then utilizing Fiji to employ MaxEntropy thresholding.

TABLE 7

| Reagent | Source | Identifier | Concentration |
|---|---|---|---|
| CD4 (D7D2Z) Rabbit mAb | Cell Signaling | 25229S | 1:200 |
| CD8α (D4W2Z) XP ® | Cell Signaling | 98941 | 1:800 |
| Rabbit mAb (mouse specific) | | | |
| Anti-TRAIL antibody | Abcam | ab231265 | 20 μg/ml |
| NKp46 (CD335) Polyclonal | Invitrogen | PA5-79720 | 1 μg/mL |
| Antibody | | | |
| FoxP3 (D6O8R) Rabbit | Cell Signaling | 12653 | 1:800 |
| mAb | | | |
| Granzyme B (E5V2L) | Cell Signaling | 44153 | 1:200 |
| Rabbit mAb | | | |
| Ki-67 (D3B5) Rabbit mAb | Cell Signaling | 12202 | 1:800 |
| (Mouse Preferred; IHC | | | |
| Formulated) | | | |
| PD-1/CD279 polyclonal | Proteintech | 18106-1-AP | 1:1000 |
| antibody | | | |
| PD-L1/CD274 monoclonal | Proteintech | 66248-1-Ig | 1:5000 |
| antibody | | | |
| Cleaved Caspase-3 (Asp175) | Cell Signaling | 9661 | 1:400 |
| antibody | | | |
| VEGF monoclonal antibody | Invitrogen | MA5-13182 | 1:20 |
| (JH121) | | | |
| TGF beta 2-specific | Proteintech | 19999-1-AP | 1:500 |
| polyclonal antibody | | | |

Microarrays. A total of 0.5×10⁶ tumor cells (HCT-116, HT-29, KM12C) were plated in a 6-well plate and allowed to adhere overnight before 24-hour treatment, as indicated. Then, 1×10 6 immune cells (NK92, TALL-104) were plated and treated with elraglusib as indicated for twenty-four h. RNA was isolated from cell pellets in batches of six using a RNeasy Plus Mini Kit (Cat #74134, Qiagen, Hilden, Germany). Acceptable RNA concentration and quality were verified with Nanodrop and Bioanalyzer measurements. GeneChip™ Human Transcriptome Array 2.0 assays were conducted according to manufacturer instructions in two batches using randomized samples to limit batch effects (Cat #902162, Applied Biosystems, Waltham, MA, USA). Applied Biosystems Transcriptomic Analysis Console (TAC) software (RRID: SCR_016519) was used to calculate fold changes in gene expression relative to the untreated control cells. Values were considered statistically significant for p values<0.05.

Single-cell RNA sequencing. Single cells were captured and 3′ single-cell gene expression libraries were conducted (Next GEM v3.1) using the 10× Genomics Chromium system by SingulOmics (SingulOmics, New York, NY, USA). Gene expression libraries were sequenced with ˜200 million PE150 reads per sample on Illumina (RRID: SCR_016387) NovaSeq (Illumina, Inc., San Diego, CA, USA). After sequencing, clean reads were then analyzed with human reference genome GRCh38 using Cell Ranger v6.1.2 (RRID: SCR_017344, 10× Genomics, Pleasanton, CA, USA). Data were analyzed and visualized using Loupe Browser (RRID: SCR_018555, 10× Genomics, Pleasanton, CA, USA).

Digital spatial profiling. An Agilent Technologies hybridization oven was used for baking tissue onto slides (Agilent, Santa Clara, CA, USA). A NanoString GeoMx® Digital Spatial Profiler (DSP) instrument (NanoString, Seattle, WA, USA) was used to scan slides, identify regions of interest (ROIs), and collect photocleavable barcodes according to manufacturer instructions. A custom panel was designed to include the following proteins: Ms IgG1, Ms IgG2a, Rb IgG, GAPDH, Histone H3, S6, Beta-2-microglobulin, CD31, CD45, Ki-67, ARG1, CD11 b, CD11 c, CD14, CD163, CD39, CD40, CD68, HLA-DR, GZMB, CD20, CD3, CD34, CD4, CD56, CD66b, CD8, Foxp3, Fibronectin, 4-1BB, B7-H3, CTLA4, GITR, IDO1, LAG3, OX40L, STING, Tim-3, VISTA, Bcl-2, ER-α, EpCAM, Her2, MART1, NY-ESO-1, PR, PTEN, PanCk, SMA, CD127, CD25, CD27, CD44, CD45RO, CD80, ICOS, PD-1, PD-L1, and PD-L2. An Eppendorf MasterCycler Gradient Thermal Cycler was used to generate the Illumina sequencing libraries from the photocleaved tags. (Eppendorf, Hamburg, Germany). An Agilent Fragment Analyzer (RRID: SCR_019417) was used for library size distribution analysis with a high-sensitivity NGS Fragment Kit (Cat #DNF-474-0500, Agilent, Santa Clara, CA, USA). qPCR for quantification was run using an Illumina-compatible KAPA Library Quantification Kits (ROX Low) (cat #KK4873) on an Applied Biosystems ViiA 7 Real-Time qPCR/PCR Thermal Cycler System (Applied Biosystems, San Francisco, CA, USA) and was analyzed using QuantStudio software (RRID: SCR_018712). Sequencing was performed using a NextSeq 500/550 High Output Kit v2.5 (75 Cycles) kit (cat #20024906) on an Illumina Sequencing NextSeq 550 System (RRID: SCR_016381, Illumina, San Diego, CA, USA). The initial annotated dataset went through quality control (QC) to check if housekeeper genes and background (isotype) control molecules were themselves correlated with the predictors of interest. Every ROI was tested for raw sequencing reads (segments with <1000 raw reads were removed), % sequencing saturation, defined as (1-deduplicated reads/aligned reads)%, segments below ˜50% were not analyzed), and nuclei count per segment, for which >100 nuclei per segment is generally recommended. Both immunoglobulins (lgGs) and housekeeper genes were highly correlated with one another. Signal to noise (SNR) ratio was calculated using background probes and all probes were detected above the background in at least one ROI. data were normalized based on background IgG expression and all normalization factors were well distributed. Data analysis and visualization were generated using R (RRID: SCR_001905; R Development Core Team, 2020).

Clinical specimens. Archival tumor specimens and peripheral blood samples were collected from patients enrolled in the Phase I study of elraglusib (Clinicaltrials.gov NCT03678883) who received treatment at the Lifespan Cancer Institute (Providence, RI, USA). The analysis was conducted in accordance with the Declaration of Helsinki and the International Conference on Harmonization Good Clinical

Practice guidelines. The study protocol was approved by the Institutional Review Board (IRB) of Rhode Island Hospital under protocol number 1324888-120. The patients also participated in a Lifespan Cancer Institute research protocol designed to investigate molecular and genetic features of tumors and mechanisms of resistance (Rhode Island Hospital IRB protocol number 449060-38). All patients provided written informed consent.

Statistical analysis. GraphPad Prism (RRID: SCR_002798) version 9.5.0 was used for statistical analyses and graphical representation (GraphPad, San Diego, CA, USA). Data are presented as means ±standard deviation (SD) or standard error of the mean (SEM). The relations between groups were compared using two-tailed, paired Student's T tests or one-way ANOVA tests. Survival was analyzed with the Kaplan-Meier method and was compared with the log-rank test. For multiple testing, Tukey's or Benjamini-Hochberg's methods were employed. Statistical significance is reported as follows: p≤0.05: *, p≤0.01: **, and p≤0.001: ***

The following EXAMPLES are provided to illustrate the invention and should not be considered to limit its scope.

### EXAMPLE 1

### GSK-3β Inhibition Elraglusib Decreases VEGF and Other Cytokines, and Boosts NK and T Cell-Mediated Killing of Colorectal Tumor Cells

A panel of human colorectal cell lines was chosen to provide a varied mutational background (TP53, KRAS, BRAF, TRK, APC, PIK3CA). Cells were treated with elraglusib at doses up to 50 μM for seventy-two hours to determine IC₁₀, IC₃₀, IC₅₀, and IC₇₀. Cytokine, chemokine, and growth factor levels were analyzed in the tumor cell culture supernatants after treatment at IC₁₀, IC₃₀, IC₅₀, and IC₇₀ for forty-eight hours using a Luminex 200 multiplexing instrument. Co-culture experiments were conducted with GFP+ SW480 colorectal cancer cells and either NK-92 natural killer cells or TALL-104 T cells at various effector/target ratios in a 48-well plate, in the presence or absence of elraglusib.

Overall cytokine levels showed a decreasing trend in response to increasing doses of elraglusib. Among the most prominently decreased growth factors in the profile was VEGF. In an immune cell-killing co-culture assay the inventors observed a significant increase in natural killer (NK-92) cell and T cell (TALL-104) killing of the colorectal cancer cells in response to treatment with elraglusib as compared to controls without drug treatment. Follow-up experiments compared the effect of pre-treating either the effector or the target cell population with elraglusib before the co-culture experiment was started. Pre-treatment of the target tumor cells, but not the effector immune cells, bolstered cell-killing, implying that the drug is sensitizing the tumor cells to killing by the immune cells. the inventors saw an increase in the expression of chemokine CXCL14 (BRAK) in the tumor cell culture supernatant with increasing doses of elraglusib. BRAK is known to stimulate activated NK cell migration and could have a beneficial therapeutic effect by increasing NK cell migration into the tumor microenvironment. The inventors saw a decrease in macrophage colony-stimulating factor (M-CSF) which, along with VEGF, has been associated with recruitment of TAMs. An elraglusib-mediated decrease of VEGF in conjunction with an elraglusib-mediated increase of BRAK secreted by the tumor cells may increase the capacity of NK-cell and T cell-mediated killing of the tumor cells. Using a compound such as elraglusib could be a way to increase the host's anti-tumor immune response to decrease tumor burden in conjunction with other therapeutic agents.

The inventors performed cell titer glo analysis to determine IC doses after treatment up to 50 μM for seventy-two hours. See TABLE below of IC₅₀ values for the three cell lines.

TABLE 8

| Cell line | 9-ING-41; IC₅₀ (μM) |
|---|---|
| HCT116 | 0.37 |
| HT29 | 0.41 |
| KM12C | 0.20 |

### EXAMPLE 2

### Circulating Biomarkers of Response to PD-1/PD-L1 Blockade and GSK-3 Inhibition

The inventors evaluated the immunomodulatory impact of elraglusib in combination with immune checkpoint blockade in a syngeneic murine model as well as in patients with refractory solid tumors of multiple tissue origins enrolled in a Phase 1 clinical trial (NCT03678883). the inventors found that murine and human plasma or serum concentrations of several circulating factors were predictive of response to PD-1/PD-L1 blockade and GSK-3 inhibition.

Using a syngeneic murine model of microsatellite colorectal cancer, the inventors evaluated GSK-3 inhibition and immune checkpoint blockade (anti-PD-1, anti-PD-L1). The inventors included the evaluation of the anti-PD-L1 therapy based on earlier preclinical observations.

The inventors found that anti-PD-L1 in combination with elraglusib was more efficacious than the combination of anti-PD-1 and elraglusib.

The inventors then performed cytokine profiling on murine serum samples. Complete and partial responders, regardless of treatment group, were more likely to have lower serum concentrations of BAFF, CCL7, CCL12, VEGF, VEGFR2, and CCL21 compared to non-responders. By contrast, complete and partial responders had higher serum concentrations of CCL4, TWEAK, GM-CSF, CCL22, and IL-12p70 compared to non-responders.

The inventors also used other methods to evaluate biomarkers, including flow cytometry (FC) and immunohistochemistry (IHC). Significant differences between responders and non-responders in intratumoral and splenic natural killer (NK) and T cell subsets 14-days post-treatment initiation were shown by multi-color flow cytometry. Compared to non-responders, regardless of treatment group, responders had lower percentages of splenic CD4+(p=0.0145) and CD8+ T cells (p=0.0001), increased percentages of splenic CD69+activated T cells (p=0.0070) and FOXP3+ regulatory T cells (p=0.001), and increased percentages of tumor-infiltrating CD3+ (p=0.0006) and CD4+ T cells (p<0.0001). Responders had lower splenic CD8+/Treg (p=0.0007) and

CD4+/Treg (p=0.001) ratios and higher intra-tumoral CD8+/Treg (p=0.0032) and CD4+/Treg (p=0.0001) ratios. Using immunohistochemistry, the inventors observed that Ki67 and TGFJ3 expression is lower in the tumors of partial responders while Granzyme B, CC3, CD4, and PD-L1 expression is higher as compared to the tumors of non-responders. Tissue-based biomarker assessment showed that partial responders had lower levels of markers of immunosuppression and tumor cell proliferation and had higher levels of markers of tumor cell apoptosis, T cell infiltration, and immune cell activation compared to non-responders.

In human samples, preliminary studies revealed that elevated pre-pharmacokinetic (PK) plasma concentrations of IL-12, Fas Ligand, IL-8, M-CSF, IL-2, IL-15, CCL7, and CCLII correlated with improved progression-free survival (PFS) in days. By contrast, decreased plasma concentrations of CXCLII and VEGF correlated with improved progression-free survival.

At the 8-hour post-PK timepoint, increased IL-8 concentrations correlated with improved progression-free survival.

At the 24-hour post-PK timepoint, increased levels of IL-12, IL-1 beta, IL-21, IL-8, IFN-alpha, IFN-gamma, M-CSF, CCL4, Fas Ligand, IL-2, IL-10, CCLII, IL-15, IL-4, and Granzyme B were correlated with improved progression-free survival.

Reduced CXCLII plasma concentrations correlated with worsened progression-free survival.

The inventors also compared pre-PK plasma concentrations of analytes with overall survival (OS). Elevated IL-8, CCLII, IFN-alpha, Fas Ligand, TRAIL R2, and IL-1 beta were correlated with improved overall survival. Decreased levels of CXCLII and TNF-alpha were correlated with improved overall survival.

At the 8-hour post-PK timepoint CCL22 and IL-8 levels were positively correlated with overall survival.

At the 24-hour post-PK timepoint IFN-alpha, Fas Ligand, TRAIL R2, and CCLII levels were positively correlated with overall survival.

### EXAMPLE 3

### Small-Molecule Inhibition of Glycogen Synthase Kinase-3 (GSK-3) Increases the Efficacy of Anti-PD-L1 Therapy in a Murine Model of Microsatellite Stable Colorectal Cancer. Therapeutic Response Correlates with T Cell Ratios and Serum Cytokine Profiles in Mice.

This EXAMPLE characterizes the effects of elraglusib in combination with immune checkpoint blockade in vivo. In a syngeneic murine colon carcinoma BALB/c model using microsatellite stable cell line CT-26, the inventors compared isotype, elraglusib (70 mg/kg 2×/week), αPD-1 (10 mg/kg 2×/week), αPD-L1 (10 mg/kg 2×/week), elraglusib+αPD-1, and elraglusib+αPD-L1 treatment groups. The median overall survival was 19 days (d) with elraglusib (hazard ratio for death (HR) 0.12; 95% confidence interval (CI), 0.03-0.54; p=0.008), 16 d for elraglusib+isotype (HR 0.40; 95% CI, 0.08-1.9; p=0.14), 20.5 d for αPD-1 (HR 0.15; 95% CI, 0.03-0.67; p=0.0185), 18 d for αPD-L1 (HR 0.21; 95% CI, 0.05-0.89; p=0.0559), 17 d for elraglusib+αPD-1 (HR 0.37; 95% CI, 0.09-0.1.5; p=0.29), and 45.5 d for elraglusib+αPD-L1 (HR 0.08; 95% CI, 0.02-0.36; p=0.0019), compared to 16 d for the isotype control group. Tumor response rates observed were 33.3% in the elraglusib+αPD-L1 group, 16.6% in the αPD-1 group, and a 0% for all other treatment groups.

Elraglusib-mediated upregulation of PD-L1 in colorectal cancer cells (HCT-116, HT-29) may be observed via both flow cytometry (FC). Western blot analysis may contribute to the increased efficacy of combination therapy with αPD-L1, as compared to αPD-1. Significant differences between responders and non-responders in intratumoral and splenic natural killer (NK) and T cell subsets 14-days post-treatment initiation were shown by multi-color FC. Compared to non-responders, regardless of treatment group, responders had lower percentages of splenic CD4+ (p=0.0145) and CD8+ T cells (p=0.0001), increased percentages of splenic CD69+ activated T cells (p=0.0070) and FOXP3+ regulatory T cells (p=0.001), and increased percentages of tumor-infiltrating CD3+ (p=0.0006) and CD4+ T cells (p<0.0001). Responders had lower splenic CD8+/Treg (p=0.0007) and CD4+/Treg (p=0.001) ratios and higher intra-tumoral CD8+/Treg (p=0.0032) and CD4+/Treg (p=0.0001) ratios. Murine serum cytokine profiling showed that responders had lower concentrations of tumorigenic cytokines (BAFF, CCL7, CCL12, VEGF, VEGFR2, CCL21) and higher concentrations of immunomodulatory cytokines (CCL4, TWEAK, GM-CSF, CCL22, IL-12p70) compared to non-responders. These results demonstrate that small-molecule inhibition of GSK-3 with elraglusib may increase the anti-tumor effects of immune checkpoint blockade and improve response in patients with microsatellite stable colorectal cancer via modulation of anti-tumor immunity and cytokine signaling.

### EXAMPLE 4

### Clinical activity of Elraglusib, a Small Molecule Selective Glycogen Synthase Kinase-3 Beta (GSK-3f3) Inhibitor, in Refractory Adult T-Cell Leukemia/Lymphoma

This EXAMPLE discloses a case of adult T-cell leukemia/lymphoma (ATLL) treated with elraglusib. A 43-year-old male patient developed diffuse lymphadenopathy. A biopsy of his axillary lymph node showed acute-type ATLL. Peripheral blood flow cytometry revealed a circulating clonal T cell population. CSF was positive for ATLL involvement.

After disease progression on the 3rd line of treatment, the patient started treatment with elraglusib monotherapy in a clinical trial (NCT03678883). CT imaging after seven months showed a partial response. Sustained reduction of peripheral blood ATLL cells lasted fifteen months. Treatment of patient-derived CD8+ T cells with elraglusib increased the secretion of IFN-γ, granzyme B, and tumor necrosis factor-related apoptosis-inducing ligand (TRAIL). In conclusion, treatment of a patient with refractory ATLL with the GSK-3β inhibitor elraglusib resulted in a prolonged response.

At eight months of therapy, a positron emission tomography (PET) scan showed new cervical, axillary, abdominal, inguinal and iliac lymphadenopathy suggestive of progressive disease which was confirmed with a biopsy of a left inguinal lymph node. Next-generation sequencing (NGS) showed a DNMT3A mutation (2.8% variant allele frequency (VAF). Peripheral blood flow cytometry confirmed persistent involvement of his ATLL (0.15×109/L).

Twenty-two months after the diagnosis of ATLL was confirmed, as a fourth-line therapy in the presence of actively progressing disease, the patient was enrolled a Phase I/II study investigating the safety and efficacy of elraglusib, a selective small molecule inhibitor of glycogen synthase kinase-3 beta (GSK-3β) as monotherapy or in combination with chemotherapy in refractory solid tumors and hematological malignancies (NCT03678883). The patient started treatment with single-agent elraglusib (12.37 mg/kg IV twice weekly: 21-day cycle). Peripheral flow cytometry prior to enrollment showed persistent involvement of ATLL (0.25×10⁹/L). The patient tolerated the treatment well without significant adverse effects.

CT imaging after two cycles showed stable disease. This stability was confirmed on CT imaging after five and seven cycles of therapy. CT imaging after ten cycles showed partial response by RECIST Criteria and continued response after nineteen cycles of treatment. Response to the treatment was also demonstrated by marked decrease in the serum concentration of soluble IL-2 receptor (sIL-2 r), which has been associated clinical activity of ATLL and can serve as a surrogate for response to therapy. Araki et al., Clin. Exp. Immunol., 119, 259-263 (2000). sIL-2 r serum concentration one month before treatment with elraglusib was 9,043 ng/ml and decreased to 2,094 ng/ml after nineteen cycles of treatment.

The patient passed away approximately six weeks from time of disease progression. Next-generation sequencing (NGS) of peripheral blood specimen showed a new TP53 mutation (c.637C>T; 70.3% VAF) and copy number analysis showed deletion of ATM (on chromosome 11q) and TP53 (chromosome 17q) and gain of NOTCH1, AKL1 (chromosome 9q).

In summary, the patient with refractory ATLL experienced a significant and durable (fifteen months) response to elraglusib following three previous lines of treatments Disease progression was associated with a newly acquired TP53 mutation.

The inventors collected peripheral blood during the patient's best response at fifteen months, corresponding with his lowest concentration of sIL-2 r in the peripheral blood, and isolated CD8+, non-ATLL T cells for further analysis. The inventors then treated these CD8+ cells ex vivo with elraglusib and assessed changes in cytokine, chemokine, and growth factor profiles in the cell culture supernatant using Luminex (LX200) technology and a custom cytokine panel described by Huntington, Louie, Zhou, & EI-Deiry, A high-throughput customized cytokinome screen of colon cancer cell responses to small-molecule oncology drugs. Oncotarget, 12, 1980-1991 (2021).

In agreement with the aforementioned observation and supporting the activation of CD8+ T cells, treatment of patient-derived CD8+ T cells with elraglusib increased the concentration of IFN-γ, granzyme B, and tumor necrosis factor-related apoptosis-inducing ligand (TRAIL) in comparison to control groups treated with DMSO.

Treatment of patient-derived CD8+ T cells with elraglusib increases granzyme B, TRAIL, and IFN-gamma secretion while decreasing VEGF, TNF-alpha, and CCL5/RANTES concentrations in cell culture supernatant in comparison to control groups. Patient-derived CD8+ cytotoxic T cells were treated with elraglusib for forty-eight hours (1 NM) or control (DMSO) and cytokine concentration was measured in cell culture supernatants. (Statistical significance was calculated with GraphPad Prism 9.3.1 using unpaired t tests and is denoted on each plot as follows: P>0.05=n.s., P≤0.05=*, P≤0.01=**, P≤0.001=***, and P≤0.0001=**** (N=3).

In view of this patient's exceptional response and preclinical results suggesting that elraglusib promotes immune activation of CD8+ T cells, an expansion cohort of patients with refractory ATLL was added to the ongoing Phase 1/2 study of elraglusib.

### EXAMPLE 5

### Plasma Cytokine Profiles and Survival Outcomes in the 1801 phase 1/2 Clinical Trial of 9-ING-41 (Elraglusib) in Patients with Advanced Cancer

Peripheral plasma samples from fifty-nine patients enrolled in the ACT1801 phase 1/2 trial of elraglusib were assayed for forty circulating cytokine levels. Patients advanced solid tumors refractory to standard treatments and received either elraglusib (1.0-15.0 mg/kg) as a single agent or in combination with other chemotherapies. Samples were taken from time points before elraglusib administration (pre-dose), after cycle 1, or after cycle 2.

Each cytokine time point combination was used to stratify patients into high and low categories using an optimal cut-point determined using maximally selected rank statistics. Kaplan Meier and Cox Proportional Hazards analysis was used to determine the efficacy of each cytokine as a biomarker. The change in cytokine values between pre-dose and cycle 1 or pre-dose and cycle 2 was evaluated in a similar manner.

A total of 142 samples spanning eight different tumor histologies were collected and analyzed. A total of 240 cytokine-timepoint combinations were evaluated. Fifty-three cytokine-timepoint combinations significantly stratified patients by overall survival (p-value, 0.05). Thirty-five of these showed that higher levels of the given cytokine correlated with a survival benefit. Eighteen showed a survival decrease.

Notable among the cytokines demonstrating survival benefits were Granzyme B at Cycle 1 Day 4 (HR: 9.54, p-value:0.015) and TGF b at Cycle 1 Day 4 (HR: 8.48, p-value:0.0063). Strong hazard ratios were also observed for cytokines that correlated with survival decreases including TRAIL-R2 at Cycle 1 Day 2 (HR: 9.56e-02, p-value:0.0001), and IL-10 at Cycle 1 Day 4 (HR: 8.77e-02, p-value:0.007).

The delta between timepoints was also able to stratify patients by overall survival.

Plasma cytokine profiling is a promising approach for discovery and validation of biomarkers prognostic for elraglusib clinical benefit.

Several cytokines measured pre-dose stratified patients into significant survival groups suggesting a potential strategy for patient enrichment in clinical trials of elraglusib.

### EXAMPLE 6

### Multiplex Digital Spatial Profiling (DSP) of Proteins in the Tumor Microenvironment in Response to GSK-3 Inhibition by Elraglusib Correlates with Immunostimulatory Effects Observed In Vivo

To determine the translational relevance of the inventors' results that showed that several circulating factors were predictive of response to PD-1/PD-L1 blockade and GSK-3 inhibition in a murine model of colorectal cancer, the inventors evaluated tumor biopsies and plasma samples from patients with refractory solid tumors of multiple tissue origins enrolled in a Phase 1 clinical trial investigating elraglusib (NCT03678883).

Plasma samples were collected from patients at baseline and twenty-four hours post-IV administration of elraglusib and were analyzed using Luminex technology. Paired FFPE tumor biopsies from patients with colorectal or pancreatic cancer before and after treatment were selected to analyze the tumor microenvironment using NanoString GeoMx DSP technology. The region of interest (ROI) selection strategy focused on mixed tumor and immune cell segments and ROIs were segmented using panCK+ and CD45+ morphology stains. Cytokine analysis revealed that elevated baseline plasma levels of IL-1 beta and reduced levels of VEGF correlated with improved progression-free survival (PFS) and overall survival (OS). Progression-free survival was also found to be positively correlated with elevated plasma levels of immunostimulatory analytes such as Granzyme B, IFN-gamma, and IL-2 at twenty-four hours post-treatment with elraglusib. CD45+ tumor-infiltrating immune cells had lower expression of VISTA, PD-1, and IDO-1 inhibitory checkpoint proteins and higher expression of OX4OL and B7-H3 stimulatory checkpoint proteins in post-treatment biopsies as compared to pre-treatment biopsies. time-on-study length negatively correlated with CD39 expression in PanCK+ segments and positively correlated with CD163 expression in CD45+ segments.

This analysis is a digital spatial analysis of tumor biopsies from patients treated with elraglusib.

These circulating biomarkers of response to GSK-3 inhibition provide significant clinical utility.

The spatial proteomics data provide insights into the immunomodulatory mechanisms of GSK-3 inhibition.

### EXAMPLE 7

### The Immunostimulatory Effect of Elraglusib in Sarcomas

Saos-2 osteosarcoma and 93T449 liposarcoma cell lines were pretreated at IC₅₀ with elraglusib. The cells were harvested for Western blot analysis after 48 hours of treatment. Both cancer cell lines as well as TALL-104 T-cells and NK-92 NK cells were treated with 0.5 μM elraglusib for twenty-four hours and harvested for Luminex cytokine profiling.

The Western blots demonstrated an increase in cPARP, an apoptotic marker, in both cancer cell lines after elraglusib treatment. An increase in PD-L1 expression was observed. The cytokine analysis revealed stimulation of immune cell activity in response to elraglusib. Treated T-cells had an increase in CXCL11, which is associated with T-cell recruitment, as well as an increased level of IL-18, which is shown to induce increased IFN-γ in Th1 cells. NK-92 cells demonstrated an increase in IL-8 chemokine and an increase in soluble TRAIL (TRAIL/TNFSF10). The cancer cell lines showed a homogenous increase in growth factor TGF-α, however, only the Saos-2 osteosarcoma cell line demonstrated an increase of IL-6. The increase in immunostimulatory cytokines as well as the increased expression of PD-L1 suggest a rationale for combining elraglusib with immune checkpoint blockade therapy. The potential synergistic effect of these two therapies is currently under investigation with co-culture experiments of sarcoma and immune cells. The treatment cohorts for these experiments include elraglusib combined with either anti-PD-L1, anti-PD-1, or anti-CTLA-4 immune checkpoint inhibitors.

### EXAMPLE 8

### Elraglusib Enhances Tumor-Infiltrating Immune Cell Activation in Tumor Biopsies and Synergize with Anti-PD-L1 in a Murine Model of Colorectal Cancer

The results of this EXAMPLE introduce several immunomodulatory mechanisms of GSK-3 inhibition using elraglusib. These results provide a rationale for the clinical evaluation of elraglusib in combination with immunotherapy.

The inventors characterized the effects of elraglusib in vitro on tumor and immune cells. The characterization included cell killing and cytokine profiling in combination with checkpoint inhibitors, in a syngeneic murine colon carcinoma BALB/c model using microsatellite stable cell line CT-26, and in human tumor biopsies and plasma samples from patients with refractory solid tumors of multiple tissue origins enrolled in a Phase 1 clinical trial investigating elraglusib (NCT03678883). The inventors used human plasma samples from patients treated with elraglusib, paired pre-treatment and post-treatment tumor biopsies and performed digital spatial proteomics.

Elraglusib promoted immune cell-mediated tumor cell killing, enhanced tumor cell pyroptosis, decreased tumor cell NF-κB-regulated survival protein expression and increased immune cell effector molecule secretion. The inventors observed synergy between elraglusib and anti-PD-L1 in an immunocompetent murine model of colorectal cancer. Murine responders had more tumor-infiltrating T-cells, fewer tumor-infiltrating Tregs, lower tumorigenic circulating cytokine concentrations, and higher immunostimulatory circulating cytokine concentrations. Murine responders had lower serum concentrations of BAFF, CCL7, CCL12, VEGF, VEGFR2, and CCL21, and higher serum concentrations of CCL4, TWEAK, GM-CSF, CCL22, and IL-12p70 as compared to non-responders. The inventors used human plasma samples from patients treated with elraglusib and correlated cytokine profiles with survival. Elevated baseline plasma levels of proteins such as IL-1 β and reduced levels of proteins such as VEGF correlated with improved progression-free survival and overall survival. Progression-free survival was also found to be positively correlated with elevated plasma levels of immunostimulatory analytes such as Granzyme B, IFN-γ, and IL-2 at twenty-four hours post-treatment with elraglusib. Several of these secreted proteins correlated with results from the in vivo analysis where expression of proteins such as IL-1 β, CCL22, CCL4, and TWEAK was positively correlated with improved response to therapy while expression of proteins such as BAFF and VEGF negatively correlated with response to therapy. Using paired tumor biopsies, the inventors found that CD45+ tumor-infiltrating immune cells had lower expression of inhibitory immune checkpoints and higher expression of T-cell activation markers in post-elraglusib patient biopsies.

### EXAMPLE 9

### Elraglusib Enhances Tumor-Infiltrating Immune Cell Activation in Tumor Biopsies and Synergize with Anti-PD-L1 in a Murine Model of Colorectal Cancer

The inventors characterized the effects of elraglusib in vitro on tumor and immune cells including cell killing and cytokine profiling, in vivo in combination with checkpoint inhibitors in a syngeneic murine colon carcinoma BALB/c model using MSS cell line CT-26, and in human tumor biopsies and plasma samples from patients with refractory solid tumors of multiple tissue origins enrolled in a Phase 1 clinical trial (NCT03678883). The inventors used human plasma samples from patients treated with elraglusib, paired pre- and post-treatment tumor biopsies and performed digital spatial proteomics.

Elraglusib promoted immune cell-mediated tumor cell killing, enhanced tumor cell pyroptosis, decreased tumor cell NF-κB-regulated survival protein expression, and increased immune cell effector molecule secretion. Synergy was observed between elraglusib and anti-PD-L1 in an immunocompetent murine model of colorectal cancer. Murine responders had more tumor-infiltrating T-cells, fewer tumor-infiltrating Tregs, lower tumorigenic circulating cytokine concentrations, and higher immunostimulatory circulating cytokine concentrations. Murine responders had lower serum concentrations of BAFF, CCL7, CCL12, VEGF, VEGFR2, and CCL21, and higher serum concentrations of CCL4, TWEAK, GM-CSF, CCL22, and IL-12p70 as compared to non-responders. The inventors used human plasma samples from patients treated with elraglusib and correlated cytokine profiles with survival. Elevated baseline plasma levels of proteins such as IL-1 β and reduced levels of proteins such as VEGF correlated with improved progression-free survival and overall survival. Progression-free survival was also found to be positively correlated with elevated plasma levels of immunostimulatory analytes such as Granzyme B, IFN-γ, and IL-2 at twenty-four hours post-treatment with elraglusib. Several of these secreted proteins correlated with results from the in vivo study where expression of proteins such as IL-1 β, CCL22, CCL4, and TWEAK was positively correlated with improved response to therapy while expression of proteins such as BAFF and VEGF negatively correlated with response to therapy. Using paired tumor biopsies, the inventors found that CD45+ tumor-infiltrating immune cells had lower expression of inhibitory immune checkpoints and higher expression of T-cell activation markers in post-elraglusib patient biopsies.

### EXAMPLE 10

### GSK-3 Inhibitor Elraglusib Enhances Tumor-Infiltrating Immune Cell Activation in Tumor Biopsies and Synergizes with Anti-PD-L1 in a Murine Model of Colorectal Cancer

Elraglusib promotes immune cell-mediated tumor cell killing of microsatellite stable colorectal cancer (CRC) cells. Mechanistically, elraglusib sensitized colorectal cancer cells to immune-mediated cytotoxicity and enhanced immune cell effector function. Using Western blots, the inventors found that elraglusib decreased colorectal cancer cell expression of NF-κB p65 and several survival proteins. Using microarrays, the inventors discovered that elraglusib upregulated the expression of proapoptotic and antiproliferative genes and downregulated the expression of cell proliferation, cell cycle progression, metastasis, TGFβ signaling, and anti-apoptotic genes in colorectal cancer cells.

Elraglusib reduced colorectal cancer cell production of immunosuppressive molecules such as VEGF, GDF-15, and sPD-L1. Elraglusib increased immune cell IFN-γ secretion, which upregulated colorectal cancer cell adermin B expression to potentially enhance pyroptosis. Elraglusib enhanced immune effector function resulting in augmented granzyme B, IFN-γ, TNF-α, and TRAIL production.

Using a syngeneic, immunocompetent murine model of microsatellite stable colorectal cancer, the inventors evaluated elraglusib as a single agent or combined with immune checkpoint blockade (anti-PD-1/L1) and observed improved survival in the elraglusib and anti-PD-L1 group. Murine responders had increased tumor-infiltrating T cells, augmented granzyme B expression, and fewer regulatory T cells. Murine responders had reduced immunosuppressive (VEGF, VEGFR2) and elevated immunostimulatory (GM-CSF, IL-12p70) cytokine plasma concentrations.

To determine the clinical significance, the inventors then used elraglusib-

treated patient plasma samples to find that reduced VEGF and BAFF and elevated IL-1 beta, CCL22, and CCL4 concentrations correlated with improved survival. Using paired tumor biopsies, the inventors found that tumor-infiltrating immune cells had a reduced expression of inhibitory immune checkpoints (VISTA, PD-1, PD-L2) and an elevated expression of T-cell activation markers (CTLA-4, OX4OL) after elraglusib treatment.

The results of this EXAMPLE address a significant gap in knowledge concerning the immunomodulatory mechanisms of GSK-3 inhibitor elraglusib, provide a rationale for the clinical evaluation of elraglusib in combination with immune checkpoint blockade, and are expected to have an impact on additional tumor types, besides colorectal cancer.

Elraglusib sensitizes tumor cells to immune-mediated cytotoxicity. A co-culture of fluorescently labeled SW480 microsatellite stable colorectal cancer cells and TALL-104 CD8+ T cells treated with elraglusib led to an increase in tumor cell death after twenty-four hours. FIG. 8A. TALL-104 cells are a human leukemic T cell line. Treatment doses were significantly less than the 24-hour IC so and 72-hour IC₅₀ calculated for all cell lines evaluated in the co-culture to ensure the majority of tumor cell death was immune-cell-mediated.

The inventors observed limited tumor cell death in SW480 monocultures treated with drug only. In the co-culture with tumor and immune cells in the absence of the drug, the baseline percentage of dead cells out of total cells was approximately 45%, after normalization. Co-cultures of tumor cells and TALL-104 T cells treated with 5 μM elraglusib had an average of 60% dead cells, while co-cultures treated with 10 μM of elraglusib had an average of 65% dead cells.

The inventors next determined the relevancy of these results using normal T cells. Donor-derived CD8+ T cells were isolated from a donor blood sample in accordance with an IRB-approved protocol. A co-culture of fluorescently labeled SW480 tumor cells and CD8+ donor-derived CD8+ T cells was then treated with elraglusib and the percentage of dead cells out of total cells was quantified after twenty-four hours. The inventors observed limited tumor cell death in SW480 monocultures treated with drug only.). The data was then normalized, as previously described. the inventors noted even more robust immune cell-mediated tumor cell death in the co-cultures treated with elraglusib. Co-cultures of tumor cells and donor-derived CD8+ T cells treated with 5 μM elraglusib had an average of 65% dead cells, while co-cultures treated with 10 μM of elraglusib had an average of 75% dead cells.

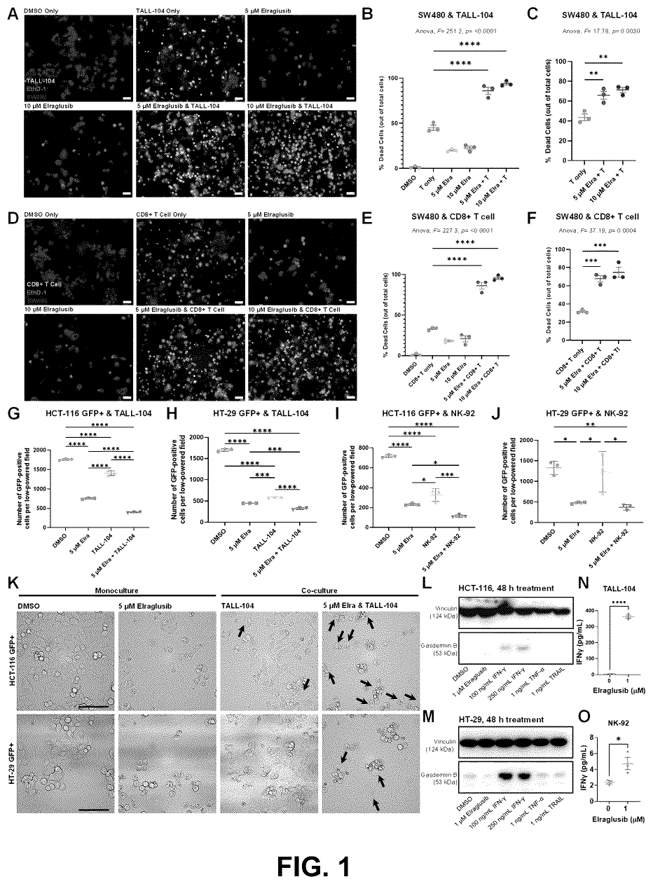

To determine if the increased amount of immune cell-mediated tumor cell killing was due to the drug's impact on the tumor cells or the immune cells, the inventors next pre-treated tumor cells with elraglusib for twenty-four hours before the co-culture with immune cells began. The inventors observed that pre-treatment with elraglusib sensitized SW480 tumor cells to TALL-104 cell-mediated tumor cell killing. FIG. 1A. The inventors used the raw percentages of cell death to normalize the data and observed minimal amounts of drug cytotoxicity at the concentration and duration of treatment used. FIG. 1B. Tumor cells pre-treated with 5 μM elraglusib for twenty-four hours and then co-cultured with TALL-104 T cells had an average of 65% dead cells. FIG. 1C. The inventors confirmed these co-culture results using donor-derived CD8+ T cells instead of TALL-104 cells. FIG. 1D. The inventors observed similar results with the CD8+ T cells where elraglusib pre-treatment of tumor cells led to a statistically significant increase in tumor cell death after twenty-four hours of co-culture. FIG. 1E. Tumor cells pre-treated with 5 μM elraglusib for twenty-four hours and then co-cultured with donor-derived CD8+ T cells had an average of 65% dead cells, while co-cultures treated with 10 μM of elraglusib for twenty-four hours and then co-cultured with donor-derived CD8+ T cells had an average of 70% dead cells. FIG. 1F.

The inventors repeated these experiments using a high-throughput GFP+ co-culture system with additional colorectal cancer cell lines HCT-116 and HT-29.The inventors evaluated both HCT-116 and HT-29 colorectal cancer cells in this co-culture model to determine if the elraglusib-mediated increase in immune cell-mediated SW480 cell killing could be reproduced in additional colorectal cancer cell lines. These cell lines were selected based on their varied mutational profiles, with both MSI-hour and MSS statuses reflected. When HCT-116 GFP+ cells were co-cultured with TALL-104 cells in the presence or absence of 5 μM elraglusib, the inventors noted a significant decrease in GFP+ cells per low-powered field in the 5 μM elraglusib only, TALL-104 only, and combination therapy groups, as compared to the DMSO only control group (FIG. 1G).

The inventors noted a significant decrease in the number of GFP+ cells per field in the combination therapy group of TALL-104 and 5 μM elraglusib co-culture condition as compared to TALL-104, which recapitulated the results observed in the first co-culture system. The inventors observed a similar trend in the HT-29 cell line, where the combination therapy group showed increased tumor cell death as compared to the drug-only or T cell-only groups (FIG. 1H).

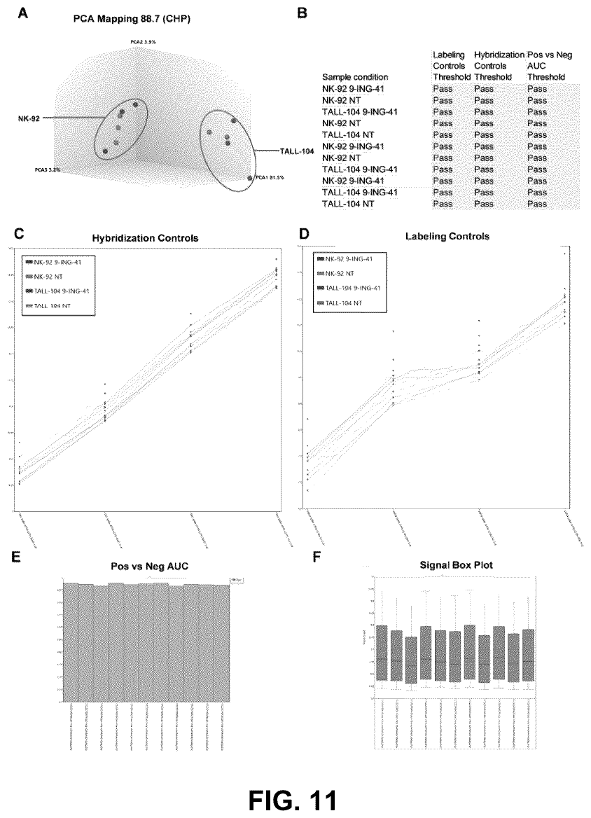

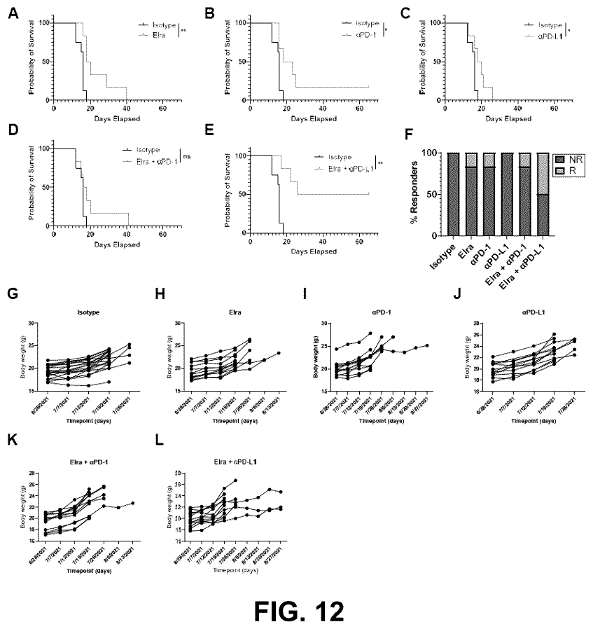

The inventors repeated the co-culture experiments with a natural killer cell line, NK-92. The inventors observed similar trends in the co-culture of NK-92 cells with HCT-116 cells, where the combination of 5 μM elraglusib and NK-92 cells showed increased tumor cell death as compared to the drug-only treatment or immune-cell-only treatment (FIG. 11). In the HT-29 cells, the inventors noted increased tumor cell death in the combination therapy group as compared to immune cells only and as compared to DMSO only. The combination therapy group did not show statistical significance when compared with elraglusib only (FIG. 1J).

Elraglusib enhances tumor cell pyroptosis in a co-culture of colorectal cancer cells and immune cells. To determine if pyroptosis-mediated immune cell activity played a role in the co-culture results, the inventors examined higher-power co-culture images for evidence of pyroptosis. The inventors observed some pyroptotic events in the co-cultures involving tumor cells and TALL-104 cells only (FIG. 1K). The inventors did not observe any pyroptotic events in the DMSO or drug-only conditions, suggesting that tumor cell pyroptosis was mediated by an immune cell-secreted molecule as it was only observed in the co-culture wells with immune cells. Interestingly, in the co-culture of colorectal cancer (HCT-116, HT-29) and TALL-104 cells in the presence of 5 μM elraglusib treatment, the inventors noted a significant increase in pyroptotic events. To determine what immune-cell-secreted molecules were most likely contributing to tumor cell pyroptosis, the inventors probed for a downstream mediator of pyroptotic death gasdermin B expression in tumor cells treated with a vehicle-only control (DMSO), 1 μM elraglusib, 100 ng/mL IFN-γ, 250 ng/mL IFN-γ, 1 ng/mL TNF-α, and 1 ng/mL TRAIL (FIG. 1L). The inventors observed an increase in gasdermin B expression with both concentrations of IFN-γ, used in both the HCT-116 cells and the HT-29 cells, suggesting that IFN-γ secreted by immune cells was a major contributor to the observed pyroptotic events (FIG. 1M). To test whether immune cells secrete more IFN-γ post-treatment with elraglusib, the inventors treated immune cell lines (TALL-104, NK-92) with elraglusib for twenty-four hours and noted a significant increase in IFN-γ post-treatment in cell culture supernatants, although this effect was significantly greater in the TALL-104 cell line as compared to the NK-92 cell line (FIG. 1N-O).

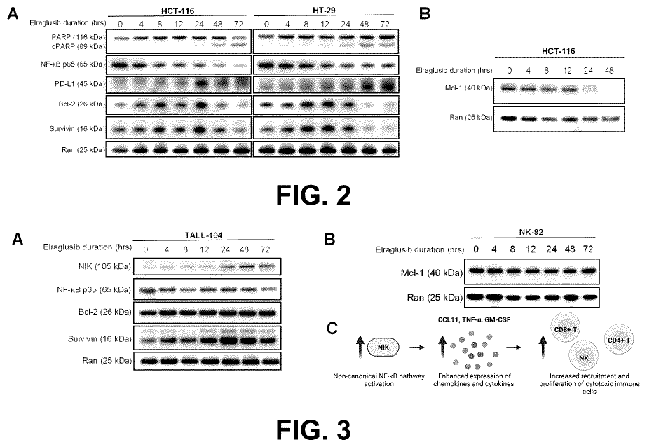

Elraglusib uprequlates tumor cell PD-L1 and proapoptotic pathway expression as well as downregulates immunosuppressive/angiogenic protein expression and pro-survival pathways. The inventors performed Western blot analyses on colorectal cancer cells (HCT-116, HT-29) treated with elraglusib over a 72-hour time course. Using the same low dose of elraglusib utilized in the co-culture assays, the inventors observed little to no cleaved PARP (cPARP) in both cell lines analyzed until the 48-hour timepoint, confirming that the tumor cell death observed in the co-culture assays was not a product of drug cytotoxicity (FIG. 2A). Because GSK-3 is a known regulator of NF-κB signaling pathways, the inventors also probed for NF-κB p65 and noted a decreased expression as the time course progressed. The inventors observed increases in PD-L1 expression as the treatment duration increased. To further elucidate the elraglusib-mediated effects on tumor cell survival, the inventors probed for survival factors Bcl-2 and Survivin and noted decreases in protein expression in both cell lines, especially at the later timepoints (forty-eight hours, seventy-two hours). In HCT-116 cells, the inventors also probed for survival factor Mcl-1 and again noted marked decreases in protein expression by the 24-hour timepoint (FIG. 2B). Although GSK-3 plays a role in the regulation of β-catenin, the inventors did not focus on elraglusib-mediated effects on β-catenin because colon cancers often harbor mutations in β-catenin or adenomatous polyposis coli (APC), thus nullifying any impact GSK-3 inhibition would have on β-catenin expression. HCT116 cells are heterozygous for β-catenin, harboring one wild-type allele and one mutant allele with inactivation of one of the residues phosphorylated by GSK-3β that is frequently mutated in tumors. Kaler, Augenlicht, & Klampfer, PLoS ONE, 7, e45462 (2012). HT-29, KM12C, and SW480 cells harbor APC mutations. Rowan et al., Proc. Natl. Acad. Sci. USA, 97, 3352 (2000). See TABLE 2.

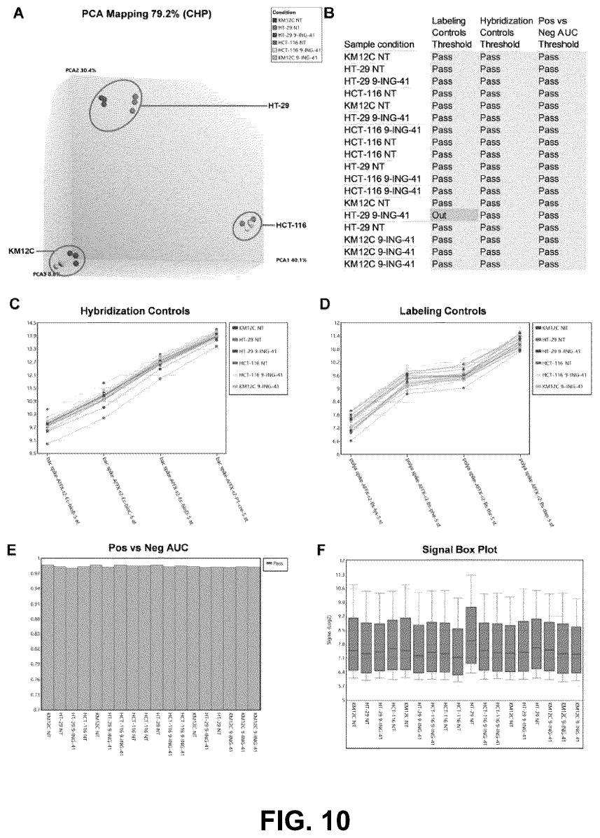

The inventors then utilized microarray analysis to gain insights into gene expression changes in colorectal cancer cell lines post-GSK-3 inhibition with elraglusib. Several colorectal cancer cell lines (HCT-116, HT-29, KM12C) were treated with elraglusib at IC-50 concentrations or DMSO as vehicle control for twenty-four hours. Treated versus untreated samples were compared in triplicate using microarray analysis FIG. 10. Results were calculated using a fold change (FC) cutoff of >1.5, <−1.5, and a minimum p-value of<0.05. HCT-116 cells had 340 differentially expressed genes post-treatment. FIG. 2C. The top differentially expressed genes of interest that were upregulated in HCT-116 cells included many anti-proliferative (BTG2, TP531NP1, LYZ, GADD45A, CDKN1A, ATF3, SESN1, SUSD6) and proapoptotic (DRAM1, FAS, BLOC1S2, TNFRSF10B, KLLN, PLK3, MXD1, GADD45B, TRIM31, TP53I3, TNFRSF10A, BAK1) genes.

TABLE 9

| Gene | Fold | | | |
|---|---|---|---|---|
| Symbol | Change | P-val | Description | Function |
| EGR1 | 5.41 | 3.81E−12 | early growth response 1 | Activates tumor supressor p53 |
| B2M | 2.41 | 6.88E−07 | beta-2-microglobulin | May shape immune landscape |
| AEN | 2.33 | 1.76E−07 | apoptosis enhancing nuclease | Proapoptotic |
| TNFRSF12A | 1.9 | 9.60E−03 | tumor necrosis factor receptor | Proapoptotic |
| | | | superfamily, member 12A | |
| TRAIP | 1.83 | 4.43E−05 | TRAF interacting protein | Regulator of NF-kappa-B |
| NCR3LG1 | 1.78 | 2.00E−03 | natural killer cell cytotoxicity | Triggers NCR3-dependent NK |
| | | | receptor 3 ligand 1 | cell activation |
| SOCS7 | 1.74 | 1.74E−06 | suppressor of cytokine | Anti-proliferative |
| | | | signaling 7 | |
| CDKN1A | 1.72 | 2.00E−04 | cyclin-dependent kinase | Anti-proliferative |
| | | | inhibitor 1A (p21, Cip1) | |
| SMAD3 | 1.69 | 7.01E−06 | SMAD family member 3 | Anti-proliferative |
| MDM4 | 1.68 | 8.02E−07 | MDM4, p53 regulator | Contributes to TP53 reguation |
| BCCIP | 1.61 | 1.24E−05 | BRCA2 and CDKN1A | Anti-proliferative |
| | | | interacting protein | |
| CCAR1 | 1.56 | 1.40E−03 | cell division cycle and | Proapoptotic |
| | | | apoptosis regulator 1; small | |
| | | | nucleolar RNA, C/D box 98 | |
| SFN | 1.54 | 1.06E−02 | stratifin | Anti-proliferative/proapoptotic |
| CRLF3 | 1.5 | 1.20E−03 | cytokine receptor-like factor 3 | Anti-proliferative |
| TNIK | −1.51 | 9.00E−04 | TRAF2 and NCK interacting | Promotes cell proliferation |
| | | | kinase | |
| BRAF | −1.53 | 2.70E−03 | B-Raf proto-oncogene, | Promotes cell proliferation |
| | | | serine/threonine kinase | |
| CD276 | −1.56 | 1.00E−04 | CD276 molecule | Suppresses antitumor activity |
| BTN3A2 | −1.58 | 4.70E−05 | butyrophilin, subfamily 3, | Inhibits the release of IFNG |
| | | | member A2 | from activated T- |
| EAPP | −1.58 | 8.36E−07 | E2F-associated | Promotes cell proliferation |
| | | | phosphoprotein | |
| JAK1 | −1.58 | 2.94E−05 | Janus kinase 1 | Promotes cell proliferation |
| PDS5B | −1.58 | 5.10E−03 | PDS5 cohesin associated | Promotes cell proliferation |
| | | | factor B | |
| MCIDAS | −1.61 | 1.46E−05 | multiciliate differentiation and | Promotes cell cycle progression |
| | | | DNA synthesis associated cell | |
| | | | cycle protein | |
| FADD | −1.66 | 5.79E−05 | Fas (TNFRSF6)-associated | Regulator of NF-kappa-B |
| | | | via death domain | |
| HIP1 | −1.67 | 6.00E−04 | huntingtin interacting protein | Antiapoptotic |
| | | | 1 | |
| IL17RA | −1.68 | 1.02E−05 | interleukin 17 receptor A | Regulator of NF-kappa-B |
| MYD88 | −1.68 | 2.00E−04 | myeloid differentiation | Regulator of NF-kappa-B |
| | | | primary response 88 | |
| CDCA3 | −1.7 | 3.00E−04 | cell division cycle associated | Promotes cell proliferation |
| | | | 3 | |
| DYNC1H1 | −1.7 | 9.20E−05 | dynein, cytoplasmic 1, heavy | Promotes cell cycle progression |
| | | | chain 1 | |
| ERBB2IP | −1.7 | 1.81E−02 | erbb2 interacting protein | Regulator of NF-kappa-B |
| FZD7 | −1.71 | 8.02E−05 | frizzled class receptor 7 | Component of the Wnt |
| | | | | signaling pathway |
| CDC45 | −1.75 | 6.40E−06 | cell division cycle 45 | Promotes cell cycle progression |
| PIM1 | −1.75 | 2.36E−05 | Pim-1 proto-oncogene, | Antiapoptotic |
| SGK1 | −1.75 | 5.24E−08 | serine/threonine kinase | Antiapoptotic |
| | | | serum/glucocorticoid | |
| | | | regulated kinase 1 | |
| UHRF1 | −1.75 | 5.00E−04 | ubiquitin-like with PHD and | Promotes cell cycle progression |
| | | | ring finger domains 1 | |
| MTA3 | −1.84 | 4.07E−09 | metastasis associated 1 family | Promotes EMT |
| | | | member 3 | |
| ITGB6 | −1.91 | 3.26E−07 | integrin beta 6 | Regulator of TGF-β Signaling |
| IL17RB | −2.03 | 6.33E−06 | interleukin 17 receptor B | Regulator of NF-kappa-B |
| AGGF1 | −2.21 | 2.00E−04 | angiogenic factor with G- | Promotes EMT |
| | | | patch and FHA domains 1 | |
| CDK2 | −2.23 | 6.11E−09 | cyclin-dependent kinase 2 | Promotes cell cycle progression |
| TNFSF15 | −2.32 | 2.58E−07 | tumor necrosis factor (ligand) | Regulator of NF-kappa-B |
| | | | superfamily, member 15 | |
| CDC25C | −2.45 | 3.43E−08 | cell division cycle 25C | Promotes cell cycle progression |
| CCNE1 | −2.6 | 1.13E−08 | cyclin E1 | Promotes cell cycle progression |
| CD14 | −2.63 | 2.82E−07 | CD14 molecule | Regulator of NF-kappa-B |
| NFKBIZ | −2.68 | 6.65E−10 | nuclear factor of kappa light | Regulator of NF-kappa-B |
| | | | polypeptide gene enhancer in | |
| | | | B-cells inhibitor, zeta | |
| CDK1 | −3.05 | 5.79E−07 | cyclin-dependent kinase 1 | Promotes cell cycle progression |
| E2F8 | −3.1 | 7.74E−07 | E2F transcription factor 8 | Promotes EMT |
| BCL6 | −3.43 | 8.65E−11 | B-cell CLL/lymphoma 6 | Antiapoptotic |
| E2F7 | −3.82 | 1.32E−07 | E2F transcription factor 7 | Promotes EMT/antiapoptotic |
| TGFBR3 | −4.82 | 1.01E−11 | transforming growth factor | Regulates TGF-β Signaling |
| | | | beta receptor III | |
| BARD1 | −5.06 | 2.65E−10 | BRCA1 associated RING | Promotes cell cycle progression |
| | | | domain 1 | |
| NFKBIA | −5.25 | 3.00E−10 | nuclear factor of kappa light | Regulator of NF-kappa-B |
| | | | polypeptide gene enhancer in | |
| | | | B-cells inhibitor, alpha | |
| CCNE2 | −5.83 | 6.53E−10 | cyclin E2 | Promotes cell cycle progression |
| MAP3K1 | −6.1 | 2.49E−13 | mitogen-activated protein | Regulator of NF-kappa-B |
| | | | kinase kinase kinase 1, E3 | |
| | | | ubiquitin protein ligase | |
| TRIB1 | −9.14 | 8.08E−12 | tribbles pseudokinase 1 | Antiapoptotic |

TABLE 9 description. HT-29 CRC cell microarray analysis. HT-29 colorectal cancer cells were treated with 1 μM elraglusib for 24 hours and treated versus untreated control samples were compared in triplicate via microarray analysis (N=3). Table showing differentially expressed genes of interest with their corresponding fold changes, p values, descriptions, and functions. Genes highlighted in yellow are known p53 targets. Genes are ordered by fold change and were calculated using a fold change cutoff of >1.5, <−1.5, and a minimum p value of <0.05.

Several of the upregulated genes are known p53 targets (BTG2, MDM2, TP531NP1, DRAM1, GADD45A, CDKN1A, PMAIP1, ATF3, FAS, SESN1, TNFRSF10D, TNFRSF10B, AEN, PLK3, TP5313, SUSD6, GDF15). Fischer, Census and evaluation of p53 target genes. Oncogene, 36, 3943-3956 (2017). Many of the downregulated genes included those that promote cell cycle progression (CDC25C, PRC1, ANLN, BARD1, PDK1, DHX32, CCNF, PRR11, TTK, FANCD2, AURκB, UHRF1), EMT (ENO2, MST1R) or cellular proliferation (FASN, ARHGEF39, FOXC1, CDCA3, MK167). Another upregulated (1.78-FC) gene of interest was PPP1R1C, and increased expression may increase tumor cell susceptibility to TNF-induced apoptosis. Zeng et al., Biotechnol. Appl. Biochem., 54, 231-238 (2009).

CMTM4 expression was downregulated (−1.84-FC) post-treatment. CMTM4 expression protects PD-L1 from being polyubiquitinated and targeted for degradation. Mezzadra et al., Nature, 549, 106-110 (2017). NEK2 was downregulated (−2.21-FC) post-treatment and NEK2 protein inhibition is known to sensitize PD-L1 blockade. Zhang et al., Nature Commun., 12, 4536 (2021).

In HT-29 cells, the inventors observed 2307 differentially expressed genes post-treatment (FIG. 2D). Many of the upregulated genes post-treatment were proapoptotic (AEN, TNFRSF12A, CCAR1, SFN) or anti-proliferative (SOCS7, CDKN1A, SMAD3, BCCIP, CRLF3). Many of the downregulated genes were involved with the promotion of cellular proliferation (TNIK, BRAF, EAPP, JAK1, PDSSB, CDCA3), cell cycle progression (MCIDAS, DYNC1H1, CDC45, UHRF1, CDK2, CDC25C, CCNE1, CDK1, BARD1, CCNE2), EMT (MTA3, AGGF1, E2F8, E2F7), or have antiapoptotic functions (PIM1, SGK1, BCL6, E2F7, TRIB1)

TABLE 10

| Gene | Fold | | | |
|---|---|---|---|---|
| Symbol | Change | P-val | Description | Function |
| IL32 | 1.55 | 4.00E−03 | interleukin 32 | Regulator of NF-kappa-B |
| GZMA | 1.53 | 2.16E−02 | granzyme A | Triggers pyroptosis |
| TNFRSF12A | 1.53 | 3.13E−02 | tumor necrosis factor receptor | Proapoptotic |
| | | | superfamily, member 12A | |
| TRAIP | 1.53 | 9.80E−03 | TRAF interacting protein | Regulator of NF-kappa-B |
| BIK | 1.51 | 1.30E−03 | BCL2-interacting killer (apoptosis- | Proapoptotic |
| | | | inducing) | |
| CDCA2 | −1.51 | 1.70E−03 | cell division cycle associated 2 | Promotes cell cycle |
| | | | | progression |
| IGF2BP2 | −1.51 | 5.00E−04 | insulin-like growth factor 2 mRNA | Promotes cell cycle |
| | | | binding protein 2 | progression/antiapoptotic |
| TRAF5 | −1.52 | 5.60E−03 | TNF receptor-associated factor 5 | Regulator of NF-kappa-B |
| CXCL1 | −1.53 | 2.00E−04 | chemokine (C-X-C motif) ligand 1 | Promotes EMT |
| MKI67 | −1.53 | 7.50E−03 | marker of proliferation Ki-67 | Promotes cell proliferation |
| TNFRSF10A | −1.54 | 6.50E−03 | tumor necrosis factor receptor | Proapoptotic/Regulator of |
| | | | superfamily, member 10a | NF-kappa-B |
| BCL9 | −1.55 | 3.49E−02 | B-cell CLL/lymphoma 9 | Promotes the Wnt signaling |
| | | | | pathway |
| CDC25C | −1.55 | 5.00E−04 | cell division cycle 25C | Promotes cell cycle |
| | | | | progression |
| TNFRSF1B | −1.56 | 2.65E−02 | tumor necrosis factor receptor | Antiapoptotic |
| | | | superfamily, member 1B | |
| CCNE1 | −1.57 | 5.79E−05 | cyclin E1 | Promotes cell cycle |
| | | | | progression |
| TRAF6 | −1.57 | 1.00E−04 | TNF receptor-associated factor 6, | Regulator of NF-kappa-B |
| BRD3 | −1.58 | 1.00E−04 | E3 ubiquitin protein ligase | Antiapoptotic |
| | | | bromodomain containing 3 | |
| CCND2 | −1.58 | 7.90E−03 | cyclin D2 | Promotes cell cycle |
| | | | | progression |
| MTBP | −1.58 | 2.92E−02 | MDM2 binding protein | Contributes to TP53 reguation |
| TNIK | −1.59 | 4.00E−04 | TRAF2 and NCK interacting | Promotes the Wnt signaling |
| | | | kinase | pathway |
| AGGF1 | −1.6 | 1.23E−02 | angiogenic factor with G-patch | Promotes EMT |
| | | | and FHA domains 1 | |
| IRF2BP2 | −1.6 | 3.00E−04 | interferon regulatory factor 2 | Promotes EMT |
| | | | binding protein 2 | |
| MDM2 | −1.61 | 1.20E−03 | MDM2 proto-oncogene, E3 | Contributes to TP53 reguation |
| | | | ubiquitin protein ligase | |
| TAB3 | −1.63 | 5.00E−04 | TGF-beta activated kinase | Regulator of NF-kappa-B |
| | | | 1/MAP3K7 binding protein 3 | |
| TNFRSF11A | −1.64 | 1.23E−05 | tumor necrosis factor receptor | Regulator of NF-kappa-B |
| | | | superfamily, member 11a, NFKB | |
| | | | activator | |
| BRCA1 | −1.71 | 8.00E−04 | breast cancer 1, early onset | Promotes cell cycle |
| | | | | progression |
| IL27RA | −1.72 | 7.00E−04 | interleukin 27 receptor, alpha | Binds immunomodulatory |
| | | | | cytokine IL-27 |
| MTDH | −1.74 | 8.48E−05 | metadherin | Regulator of NF-kappa-B |
| FZD3 | −1.76 | 8.90E−05 | frizzled class receptor 3 | Component of the Wnt |
| | | | | signaling pathway |
| MET | −1.79 | 1.77E−05 | MET proto-oncogene, receptor | Promotes EMT |
| | | | tyrosine kinase | |
| ERBB2IP | −1.88 | 9.00E−04 | erbb2 interacting protein | Regulator of NF-kappa-B |
| IL6ST | −1.88 | 8.13E−05 | interleukin 6 signal transducer | Component of the IL-6 |
| | | | | signaling pathway |
| NRP1 | −1.88 | 1.70E−05 | neuropilin 1 | Promotes EMT |
| MAP3K1 | −1.92 | 1.31E−05 | mitogen-activated protein kinase | Regulator of NF-kappa-B |
| | | | kinase kinase 1, E3 ubiquitin | |
| | | | protein ligase | |
| CDK1 | −2 | 1.00E−04 | cyclin-dependent kinase 1 | Promotes cell cycle |
| | | | | progression |
| JMY | −2.03 | 1.15E−05 | junction mediating and regulatory | Contributes to TP53 reguation |
| | | | protein, p53 cofactor | |
| BRAF | −2.05 | 2.00E−04 | B-Raf proto-oncogene, | Promotes cell proliferation |
| | | | serine/threonine kinase | |
| CD109 | −2.09 | 3.00E−04 | CD109 molecule | Regulates TGF-β Signaling |
| TGFBR2 | −2.11 | 7.17E−07 | transforming growth factor beta | Regulates TGF-β Signaling |
| | | | receptor II | |
| LTBP1 | −2.24 | 4.70E−06 | latent transforming growth factor | Regulates TGF-β Signaling |
| | | | beta binding protein 1 | |
| TLR3 | −2.25 | 6.24E−06 | toll-like receptor 3 | Regulator of NF-kappa-B |
| GDF15 | −2.29 | 6.00E−04 | growth differentiation factor 15 | Promotes EMT |
| TRIB1 | −2.37 | 8.89E−06 | tribbles pseudokinase 1 | Antiapoptotic |
| E2F7 | −2.5 | 9.13E−05 | E2F transcription factor 7 | Promotes EMT/antiapoptotic |
| NFKBIA | −2.51 | 3.25E−07 | nuclear factor of kappa light | Regulator of NF-kappa-B |
| | | | polypeptide gene enhancer in B- | |
| | | | cells inhibitor, alpha | |
| CCNE2 | −2.64 | 5.46E−07 | cyclin E2 | Promotes cell cycle |
| | | | | progression |
| BARD1 | −3.03 | 1.59E−07 | BRCA1 associated RING domain | Promotes cell cycle |
| | | | 1 | progression |
| E2F8 | −3.16 | 3.26E−07 | E2F transcription factor 8 | Promotes EMT |
| MAP2K6 | −3.66 | 1.13E−09 | mitogen-activated protein kinase | Antiapoptotic |
| | | | kinase 6 | |
| TGFBR3 | −3.72 | 7.53E−10 | transforming growth factor beta | Regulates TGF-β Signaling |
| | | | receptor III | |

TABLE 10 description. KM12C colorectal cancer cell microarray analysis. KM12C colorectal cancer cells were treated with 1 μM elraglusib for 24 hours and treated versus untreated control samples were compared in triplicate via microarray analysis (N=3). Table showing differentially expressed genes of interest with their corresponding fold changes, p values, descriptions, and functions. Genes highlighted in yellow are known p53 targets. Genes are ordered by fold change and were calculated using a fold change cutoff of >1.5, <−1.5, and a minimum p value of <0.05.

NCR3LG1 (B7-H6) was upregulated (1.78-FC) post-treatment. NCR3LG1 triggers NCR3-dependent NK cell activation and cytotoxicity. Brandt et al., J. Exp. Med., 206, 1495-1503 (2009).

KM12C cells had 1032 differentially expressed genes post-treatment (FIG. 2E). The inventors observed an upregulation of proapoptotic genes (TNFRSF12A, BIK). The inventors observed a downregulation of genes involved in the promotion of EMT (CXCL1, AGGF1, IRF2BP2, MET, NRP1, GDF15, E2F8), the promotion of cell cycle progression (CDCA2, IGFBP2, CDC25C, CCNE1, CCND2, CDK1, CCNE2, BARD1), cellular proliferation (MK167, BRAF), and the regulation of TGFβ signaling (TGFBR2, LTBP1, TGFBR3, CD109) in KM12C cells post-treatment as compared to control.

The inventors noticed an upregulation (1.53-FC) of GZMA (granzyme A) expression post-treatment, which is known to cleave gasdermin B to induce pyroptosis. Zhou et al., Science, 368, 2-7 (2020).

Several relevant signaling pathways had differentially expressed genes post-elraglusib in all three cell lines including the VEGFA-VEGFR2, TGFβ, IL-18, CCL18, EGF/EGFR, miR-targeted genes in lymphocytes, apoptosis, and cell cycle signaling pathways (FIG. 2F). The most significant commonly downregulated signaling pathway was VEGFA-VEGFR2, which had 29 downregulated genes in HCT-116 cells, 37 downregulated genes in HT-29 cells, and forty-eight downregulated genes in KM12C cells. A Venn diagram was used to compare the 3124 genes that were differentially expressed post-treatment with elraglusib as compared to control in the three colon cancer cell lines (HCT-116, HT-29, KM12C) (FIG. 2G). HCT-116 cells had 241 (7.7%), HT-29 cells had 1805 (57.8%) and KM12C cells had 549 (17.6%) differentially expressed genes post-treatment as compared to control. HCT-116 and HT-29 cells shared 46 differentially expressed genes (1.5%), HCT-116 and KM12C shared 27 (0.86%), KM12C and HT-29 shared 430 (13.8%), and all three cell lines shared 26 (0.83%). All three cell lines showed a post-treatment differential expression of NF-κB regulators with increased expression of many negative regulators of NF-κB (NFκBIA, TNFAIP3, TRAIP, IL32) and decreased expression of several positive regulators of NF-κB (IRAK1BP1, FADD, IL17RA, MYD88, ERBB2IP, IL17RB, TNFSF15, NFκBIZ, NFκBIA, MAP3K1, TRAF5, TRAF6, TAB3, TNFRSF11A, MTDH, TLR3).

The inventors previously found that elraglusib treatment of human colorectal cancer cell lines (HCT-116, HT-29, KM12C) with varied mutational profiles modified cytokine, chemokine, and growth factor secretion into cell culture media. Huntington, Louie, Zhou, & EI-Deiry, Oncotarget, 12, 1980-1991 (2021). The inventors in this EXAMPLE treated tumor cells (HCT-116, HT-29) with 1 μM or 5 μM elraglusib for forty-eight hours and subsequently analyzed the cell culture supernatant using Luminex 200 technology (FIG. 2H,I). Several cytokines, chemokines, and growth factors associated with angiogenesis or EMT were downregulated in both cell lines (HCT-116, HT-29) at both concentrations of elraglusib. Notably, GDF-15, GM-CSF, and VEGF all had decreased secretion post-treatment in both cell lines and at both concentrations of elraglusib. Several cytokines, chemokines, and growth factors associated with immunosuppression were also downregulated post-treatment, including CCL5/RANTES, DcR3, Fas, and soluble PD-L1 (sPD-L1).

Elraglusib enhances immune cell effector function. the inventors next analyzed immune cell lines (TALL-104, NK-92) using western blot analysis. Interestingly, when the inventors probed for the same proteins in the cytotoxic immune cell lysates, the inventors observed many opposing trends to those observed in the tumor cells. In TALL-104 cells, the inventors did not notice significant changes in NF-κB or survival protein BcI-2 as treatment duration increased (FIG. 3A).

Because of the differential impact of elraglusib on tumor and immune cells that the inventors observed via Western blot, the inventors compared the levels of another survival protein Mcl-1 in NK-92 natural killer cells. The inventors did not observe a significant decrease in Mcl-1 protein expression through the 72-hour endpoint (FIG. 3B). Surprisingly, the inventors noted increases in survival protein Survivin and NF-κB-inducing kinase (NIK), a protein commonly associated with activation of the non-canonical NF-κB signaling pathway, which led us to create a working model of NIK-mediated immune cell recruitment (FIG. 3C).

Microarray analysis was used to obtain insights into gene expression changes in immune cell lines post-GSK-3 inhibition with elraglusib. Immune cell lines (TALL-104, NK-92) were treated with elraglusib at IC₅₀ concentrations or DMSO as vehicle control for twenty-four hours and treated versus untreated samples were compared in triplicate using microarray analysis. FIG. 11. Results were calculated using a fold-change (FC) cutoff of >1.5, <−1.5, and a minimum p-value of <0.05. NK-92 cells had sixty-one differentially expressed genes post-treatment (FIG. 3D). the inventors observed an increase in genes that promote immune cell proliferation (TNFSF14, RAB38) and control immune cell adhesion and migration (WNK1) post-elraglusib treatment.

TABLE 11

| Gene | Fold | | | |
|---|---|---|---|---|
| Symbol | Change | P-val | Description | Function |
| RNY5 | 3.32 | 0.027 | RNA, Ro-associated Y5 | May modulate NF-κB activity |
| RAB38 | 1.65 | 5.44E− | RAB38, member RAS | Promotes cellular proliferation |
| | | 05 | oncogene family | |
| TNFSF14 | 1.5 | 0.043 | tumor necrosis factor (ligand) | Stimulates T cell proliferation |
| | | | superfamily, member 14 | |
| WNK1 | 1.5 | 0.0119 | WNK lysine deficient protein | Controls immune cell adhesion and |
| | | | kinase 1 | migration |
| NAP1L1 | −1.51 | 0.0278 | nucleosome assembly protein | May modulate NF-κB activity |
| | | | 1-like 1 | |
| MIR186 | −1.52 | 0.0132 | microRNA 186 | Proapoptotic |
| ITGB8 | −1.53 | 0.0059 | integrin beta 8 | Activates latent TGFβ to supress |
| | | | | immune responses |
| S100A12 | −1.78 | 0.0407 | S100 calcium binding protein | Proapototic |
| | | | A12 | |

TABLE 11 description. NK-92 immune cell microarray analysis. NK-92 immune cells were treated with 1 μM elraglusib for 24 hours and treated versus untreated control samples were compared in triplicate via microarray analysis (N=3).

Table showing differentially expressed genes of interest with their corresponding fold changes, p values, descriptions, and functions. Genes are ordered by fold change and were calculated using a fold change cutoff of >1.5, <−1.5, and a minimum p value of <0.05.

The inventors also noted decreases in proapoptotic genes (MIR186, S100A12) and genes involved in the activation of latent TGF to suppress immune cell function (ITGB8). TALL-104 cells had sixty-four differentially expressed genes post-treatment (FIG. 3E). the inventors observed an increased expression of genes involved in the modulation of NF-κB activity (RNY4, RNYS), cytotoxic granule exocytosis (STX19, VAMPS), and anti-apoptotic gene BCL2-related protein A1 (BCL2A1).

TABLE 12

| Gene | Fold | | | |
|---|---|---|---|---|
| Symbol | Change | P-val | Description | Function |
| RNY4 | 2.19 | 0.0218 | RNA, Ro-associated Y4 | May modulate NF-κB activity |
| RNY5 | 1.66 | 0.0416 | RNA, Ro-associated Y5 | May modulate NF-κB activity |
| BCL2A1 | 1.63 | 0.023 | BCL2-related protein A1 | Antiapoptotic |
| CKS1B | 1.6 | 0.013 | CDC28 protein kinase | Promotes cell proliferation |
| STX19 | 1.58 | 0.0004 | regulatory subunit 1B | Involved in cytotoxic granule |
| | | | syntaxin 19 | exocytosis |
| VAMP8 | 1.56 | 0.0193 | vesicle associated membrane | Involved in cytotoxic granule |
| | | | protein 8 | exocytosis |
| KIF7 | 1.56 | 0.0091 | kinesin family member 7 | Required for T cell development and |
| | | | | MHC expression |
| CCL3 | 1.52 | 0.022 | chemokine (C-C motif) ligand | Recruits and enhances proliferation of |
| | | | 3 | CD8+ T cells |
| ORAI3 | 1.51 | 0.0387 | ORAI calcium release- | Promotes cell proliferation |
| | | | activated calcium modulator 3 | |
| CD84 | −1.53 | 0.0318 | CD84 molecule | Regulator of immune cell function |
| PPARA | −1.54 | 0.0074 | peroxisome proliferator- | Regulator of immune cell function |
| | | | activated receptor alpha | |
| PTPN3 | −1.56 | 0.0239 | protein tyrosine phosphatase, | Inhibitory immune checkpoint |
| | | | non-receptor type 3 | |
| ACVR1B | −1.61 | 0.0205 | activin A receptor type IB | Regulates TGFβ signaling |
| PTPN14 | −1.62 | 0.0041 | protein tyrosine phosphatase, | Regulates TGFβ signaling |
| | | | non-receptor type 14 | |
| HSPA1A | −1.63 | 0.0391 | heat shock 70 kDa protein 1B; | Proapoptotic |
| | | | heat shock 70 kDa protein 1A | |
| DUSP6 | −1.66 | 0.0013 | dual specificity phosphatase 6 | Regulator of immune cell function |
| UBE3A | −1.73 | 0.0286 | ubiquitin protein ligase E3A | Proapoptotic |
| CCR8 | −2.14 | 0.0004 | chemokine (C-C motif) | Marker of regulatory T cells |
| | | | receptor 8 | |
| CAMK1D | −2.5 | 0.0256 | calcium/calmodulin-dependent | Key modulator of tumor-intrinsic |
| | | | protein kinase ID | immune resistance |

TABLE 12 description. TALL-104 immune cell microarray analysis. TALL-104 immune cells were treated with 1 μM elraglusib for 24 hours and treated versus untreated control samples were compared in triplicate via microarray analysis (N=3). Table showing differentially expressed genes of interest with their corresponding fold changes, p values, descriptions, and functions. Genes are ordered by fold change and were calculated using a fold change cutoff of >1.5, <−1.5, and a minimum p value of <0.05.

The inventors also saw an upregulation (1.56-FC) of kinesin family member 7 (KIF7), which is required for T cell development and MHC expression, as well as an increased expression (1.52-FC) of chemokine C-C motif ligand 3 (CCL3) which is known to recruit and enhance the proliferation of CD8+ T cells. Honey, Nature Rev. Immunol., 6, 427 (2006). The inventors observed a decreased expression of genes involved in TGF signaling pathways (ACVR1B, PTPN14) and proapoptotic genes (HSPA1A, UBE3A). the inventors also saw a decreased expression (−1.56-FC) of inhibitory immune checkpoint protein tyrosine phosphatase, non-receptor type 3 (PTPN3). In total, there were 124 differentially expressed genes post-treatment and only 1, an unnamed gene (probe set ID TC22000564.hg.1, coding), was shared between both cell lines (FIG. 3F).

To determine if there was any heterogeneity in response to drug treatment, the inventors employed a 10× single-cell sequencing analysis on both immune cell lines (TALL-104, NK-92) treated with low-dose 1 μM elraglusib or vehicle control (DMSO) for twenty-four hours. Samples clustered by cell type when aggregate data were visualized using a t-SNE plot (FIG. 3G). Interestingly, immune cells showed a differential expression of mitochondrial-encoded genes (MD and ribosomal genes (RB) post-treatment with elraglusib, suggesting a metabolic shift in line with the extensive metabolic reprogramming undertaken in immune cells post-activation to support immune cell activities such as cytokine production. See Chou, Rampanelli, Li, & Ting, Cell. Mol. Immunol., 19, 337-351 (2022). (FIG. 3H). Several genes showed the same trends post-treatment in both cell lines (FIG. 3I). In both cell lines, the inventors observed an increase in immune cell activation marker CD69 and a decrease in the immunosuppressive marker CHI3L1. The inventors noted an increase in immune cell attractant CCL4 in the NK-92 cells and an increase in immune cell chemoattractant CXCR4 in the TALL-104 cells.

Because the previously observed non-canonical NF-κB pathway activation enhances the expression of immune cell chemotactic chemokines and cytokines, the inventors determine how elraglusib treatment impacts the immune cell secretome. TALL-104 and NK-92 cells were treated with 1 μM elraglusib for forty-eight hours before cell culture supernatant was collected for cytokine profile analysis. TALL-104 cells treated with elraglusib showed increases in effector molecules IFN-γ, granzyme B, and TRAIL concentrations, as measured in picogram per milliliter (FIG. 3J). NK-92 cells treated with elraglusib showed increases in IFN-γ and TRAIL but decreases in the concentration of secreted granzyme B.

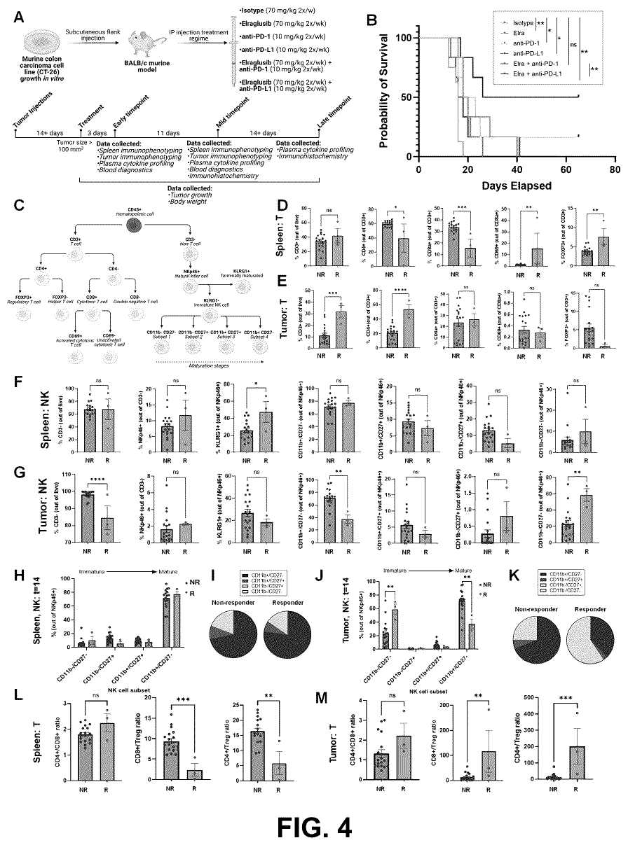

Elraglusib significantly prolongs survival in combination with anti-PD-L1 therapy in a syngeneic microsatellite stable colorectal cancer murine model. The inventors evaluated the potential for elraglusib to increase the efficacy of immune checkpoint blockade and utilized a syngeneic murine colon carcinoma BALB/c murine model using a MSS cell line CT-26 (FIG. 4A). Mice were randomly assigned to one of seven groups: isotype (N=12), elraglusib (N=12), elraglusib+isotype (N=12), anti-PD-1 (N=12), anti-PD-L1 (N=12), elraglusib+anti-PD-1 (N=12), and elraglusib+ anti-PD-L1 (N=12). In this MSS colorectal cancer model, the inventors observed a significantly improved survival curve in the elraglusib and anti-PD-L1 combination therapy group (FIG. 4B). the inventors also observed statistically significant improved survival in the elraglusib, anti-PD-1, and anti-PD-L1 alone groups as compared to the control. See FIG. 12. The inventors saw the most sustained response in the elraglusib and anti-PD-L1 combination therapy group. See FIG. 12. Murine body weights did not differ significantly regardless of the treatment group. See FIG. 12. The mice did not show evidence of significant treatment-related toxicity on complete blood count or serum chemistry analysis.

TABLE 13

| | Isotype | | | | αPD-1 + | αPD-L1 + |
|---|---|---|---|---|---|---|
| Test code | control | Elraglusib | αPD-1 | αPD-L1 | Elraglusib | Elraglusib |
| BUN (mg/dL) | 23 | 27 | 28 | 23 | 23 | 23 |
| CREA (mg/dL) | 0.2 | 0.2 | 0.2 | <0.2 | 0.2 | 0.2 |
| GLU (mg/dL) | 217 | 230 | 340 | 176 | 240 | 292 |
| NA (mmol/L) | 148 | 146 | 146 | 149 | 147 | 147 |
| K (mmol/L) | 6.8 | >10.0 | 6.1 | 6.9 | 6.2 | 5.7 |
| CL (mmol/L) | 110 | 111 | 112 | 116 | 111 | 114 |
| ALP (U/L) | 38 | 4 | 46 | 21 | 36 | 83 |
| ALT (U/L) | 16 | 26 | 23 | 42 | 26 | 36 |
| AST (U/L) | 266 | 198 | 197 | 883 | 217 | 218 |
| TBIL (mg/dL) | 0.2 | 0.5 | 0.1 | 0.4 | 0.2 | 0.1 |
| DBIL (mg/dL) | 0 | 0 | 0 | 0.1 | 0 | 0 |
| LDH (U/L) | 867 | 2230 | 710 | >12000 | 771 | 447 |
| CPK (U/L) | 476 | 1968 | 1396 | 447 | 653 | 1477 |
| GGT (U/L) | 0 | 0 | 0 | 0 | 0 | 0 |
| TPRO (g/dL) | 4.2 | 4.5 | 4.2 | 3.7 | 4.2 | 4.3 |
| ALB (g/dL) | 2.4 | 2.7 | 2.4 | 2 | 2.5 | 2.6 |
| CA (mg/dL) | 10 | 0.4 | 9.6 | 7.3 | 10.2 | 8.6 |
| PHOS (mg/dL) | 8.4 | 8.2 | 11.4 | 6.2 | 7.5 | 8.9 |
| MG (mg/dL) | 2.5 | 1.2 | 2.9 | 2.1 | 2.5 | 2.5 |
| CHOL (mg/dL) | 90 | 101 | 81 | 81 | 83 | 67 |
| TRIG (mg/dL) | 269 | 136 | 159 | 434 | 169 | 75 |
| AMY (U/L) | 265 | 275 | 307 | 241 | 318 | 442 |
| LIP (U/L) | 49 | 59 | 55 | 52 | 42 | 66 |
| WBC (10³/μL) | — | — | 2.96 | 1.8 | 3.75 | 2.68 |
| RBC (10⁶/μL) | — | — | 8.86 | 8.16 | 8.5 | 8.85 |
| HB (g/dL) | — | — | 13 | 11.9 | 12.7 | 13.2 |
| HCT (%) | — | — | 50.6 | 46.9 | 49 | 51 |
| MCV (fL) | — | — | 57.1 | 57.5 | 57.6 | 57.6 |
| MCH (pg) | — | — | 14.6 | 14.6 | 14.9 | 14.9 |
| MCHC (g/dL) | — | — | 25.6 | 25.3 | 25.9 | 25.8 |
| PLT (10³/μL) | — | — | 403 | 691 | 401 | 471 |
| NEU % (%) | — | — | 55.8 | 29.4 | 60.1 | 50.8 |
| NEU (10³/μL) | — | — | 1.65 | 0.53 | 2.26 | 1.36 |
| LYM % (%) | — | — | 34.7 | 57.3 | 33.1 | 39.6 |
| LYM (10³/μL) | — | — | 1.03 | 1.03 | 1.24 | 1.06 |
| MON % (%) | — | — | 3 | 3.3 | 1.2 | 2.2 |
| MON (10³/μL) | — | — | 0.09 | 0.06 | 0.04 | 0.06 |
| EOS % (%) | — | — | 3 | 5.1 | 2.8 | 4.3 |
| EOS (10³/μL) | — | — | 0.09 | 0.09 | 0.11 | 0.11 |
| BAS % (%) | — | — | 0.8 | 0.5 | 0.3 | 1.4 |
| BAS (10³/μL) | — | — | 0.02 | 0.01 | 0.01 | 0.04 |
| LUC % (%) | — | — | 2.6 | 4.4 | 2.5 | 3.2 |
| LUC (10³/μL) | — | — | 0.08 | 0.08 | 0.1 | 0.09 |

TABLE 13 description. Murine serum chemistry analysis does not show treatment-related toxicity. Whole blood from long-term mice sacrificed was submitted for serum chemistry analysis. Results are shown from the complete metabolic panel and complete blood count with differential. ALB: Albumin, ALP: Alkaline Phosphatase, ALT: Alanine aminotransferase, AMY: Amylase, ANIS: Anisocytosis, AST: Aspartate aminotransferase, ATYP: Atypical Lymphs, BAS: Absolute Basophils, BAS%: % Basophils, BUN: Urea Nitrogen, CA: Calcium, CHOL: Cholesterol, CL: Chloride, CPK: Creatine kinase, CPLT: Clumped Platelets, CREA: Creatinine, DBIL: Direct Bilirubin, EOS: Absolute Eosinophils, EOS% : % Eosinophils, GGT: Gamma-glutamyl Transferase, GLU: Glucose, HB: Hemoglobin, HCT: Hematocrit, HJB: Howell-Jolly Bodies, HYPO: Hypochromasia, HYPR: Hyperchromasia, K: Potassium, LDH: Lactate Dehydrogenase, LIP: Lipase, LPLT: Large Platelets, LUC: Absolute Large Unstained Cells, LUC%: % Large Unstained Cells, LYM: Absolute Lymphocytes, LYM%: % Lymphocytes, MAC: Macrocytosis, MCH: Mean Corpuscular Hemoglobin, MCHC: Mean Corpuscular Hemoglobin Count, MCV: Mean Corpuscular Volume, MG: Magnesium, MIC: Microcytosis, MON: Absolute Monocytes, MON%: % Monocytes, NA: Sodium, NEU: Absolute Neutrophils, NEU%: % Neutrophils, PHOS: Inorganic Phosphorus, PLT: Platelet Count, POLK: Poikilocytosis, RBC: Red Blood Cell Count, TBIL: Total Bilirubin, TPRO: Total Protein, TRIG: Triglyceride, WBC: White Blood Cell Count.

Both the elraglusib individual treatment and dual treatment groups maintained normal renal function, as evidenced by normal blood urea nitrogen (BUN) and creatinine and were free of significant electrolyte perturbations. Liver function tests did not reveal any evidence of liver toxicity and the dual-treatment mice did not have any elevations in AST, ALT, or bilirubin. As can be expected in mice with significant tumor burdens, mice across treatment groups had decreased albumin levels and evidence of mild marrow hypoplasia resulting in mild anemia and reduced white blood cell and platelet counts. This effect was independent of the treatment group and likely related to tumor burden at the time of sacrifice.

Murine responders have more T Cell tumor-infiltration and elevated tumoral CD8+/Treg and CD4+/Treg ratios. The inventors used multi-color flow cytometry to characterize the natural killer (NK) and T cell populations two weeks after treatment initiation, and immune cell subpopulations were analyzed in both the spleen and the tumor (FIG. 4C). Two weeks after treatment initiation, mice were grouped as responders (R) or non-responders (NR) based on a tumor volume less than or greater than 100 mm3, respectively. Compared to non-responders, regardless of treatment group, responders two weeks after treatment had statistically significantly reduced levels of splenic CD4+ and CD8+ T cells and had increased percentages of CD69+ activated T cells and Foxp3+ regulatory T cells (Tregs) (FIG. 4D). Responders had increased percentages of tumor-infiltrating CD3+ and CD4+T cells (FIG. 4E). The inventors also observed that responders had increased percentages of splenic KLRG1+ mature NK cells and tumor-infiltrating CD11b−/CD27− immature NK cells, and decreased percentages of tumor-infiltrating CD11b+/CD27− activated NK cells two weeks after treatment initiation (FIG. 4F,G). the inventors did not observe striking differences between non-responders and responders in the splenic immature natural killer cell subsets (CD11b−/CD27−, CD11b−/CD27+, CD11b+/CD27+, CD11b+CD27−) (FIG. 4H,I). The inventors did observe significant differences between non-responders and responders in the tumor-infiltrating immature natural killer cell subsets (FIG. 4J,K). the inventors observed that responders had a greater proportion of immature (CD11b−/CD27−) NK cells and a reduced proportion of mature (CD11b+CD27−) NK cells two weeks after treatment initiation. When comparing the T cell ratios, compared to non-responders, responders had a reduced splenic CD8+/Treg and CD4+/Treg ratio (FIG. 4L). The CD8+/Treg ratio is commonly used as an index of TIL's effector function. Sideras et al., J. Surg. Oncol., 118,68-76 (2018). The responders had an elevated intra-tumoral CD8+/Treg and CD4+/Treg ratio (FIG. 4M). The observed changes in immune cell subsets in responders are consistent with the increased infiltration of cytotoxic immune cells into the tumor.

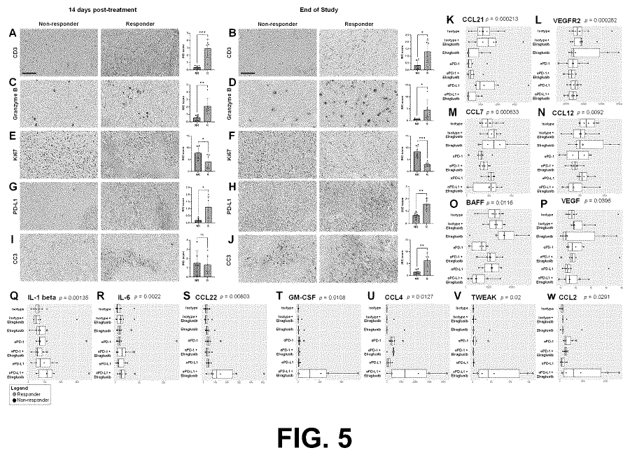

Murine responders show an immunostimulatory tumor microenvironment by immunohistochemistry. The inventors conducted immunohistochemistry (IHC) on tumor sections from the two weeks post-treatment initiation timepoint or from the end-of-study (EOS) timepoint. the inventors compared non-responders (NR) and responders (R) and stained for T cell marker CD3 and observed that responders had significantly more CD3+ T cells as compared to non-responders at both timepoints. (FIG. 5A,B). To determine if there were any differences in immune cell activation, the inventors stained for Granzyme B and again observed that responders had significantly more Granzyme B+ staining at both timepoints as compared to non-responders (FIG. 5C,D). the inventors stained for Ki67 as a marker of tumor cell proliferation and observed that responders had less tumor cell proliferation as compared to responders at both timepoints (FIG. 5E,F). As the inventors found that elraglusib upregulated tumor cell PD-L1 expression and observed an improvement in survival when elraglusib was combined with anti-PD-L1 therapy as compared to anti-PD-1 therapy, the inventors next looked at PD-L1 staining in the tumor sections (FIG. 5G,H). The inventors observed that responders had more PD-L1+ tumor cells than non-responders at both timepoints. To examine tumor cell apoptosis, the inventors then stained for cleaved-caspase 3 (CC3) and noted that there was no difference in CC3 expression at the mid-timepoint (two weeks after treatment initiation); however, responders did have significantly elevated CC3 expression than non-responders at the EOS timepoint (FIG. 51,4 the inventors also analyzed the expression of CD4, CD8, Foxp3+, NKp46, TRAIL, PD-1, VEGF, and TGFβ2 to gain additional insights into the tumor immune microenvironment at both the two weeks after treatment initiation timepoint and the EOS timepoint, respectively. To examine helper T cell presence, the inventors stained for CD4 and observed that responders had more CD4+ T cells than non-responders at both timepoints. Interestingly, the inventors saw the same trends when the inventors examined CD8 expression, where responders had more CD8+ T cells than non-responders, which differed from the flow cytometry results but could be explained by the large variability in CD8a+ T cells the inventors observed using flow cytometry in the non-responder group. the inventors did not observe statistically significant differences in Foxp3+ Treg expression between responders and non-responders at either timepoint. When the inventors examined NK cell tumor-infiltration by immunohistochemistry, the inventors noted more NKp46+ NK cells in responders at the two weeks after treatment initiation timepoint, but this difference was not significant at the EOS timepoint, The inventors examined another cytotoxic mediator, TRAIL, and observed no difference between responders and non-responders at the mid-timepoint. Interestingly, the inventors observed reduced TRAIL expression in the responders as compared to the non-responders at the EOS timepoint. The inventors also examined PD-1 expression and did not note any significant differences between responders and non-responders at either of the timepoints. The inventors noted a similar lack of significance when they examined immunosuppressive and angiogenic VEGF expression. The inventors examined immunosuppressive TGFβ2 expression and noted no differences between responders and non-responders at the mid-timepoint but noted that responders had significantly reduced expression at the EOS timepoint.

Murine responders have reduced tumorigenic and elevated immunomodulatory cytokine concentrations. The inventors next analyzed murine serum samples from EOS mice for cytokine profiles and noted interesting trends between responders and non-responders. Responders were more likely to have reduced serum concentrations of CCL21 (p=0.000213), VEGFR2 (p=0.000282), CCL7 (p=0.000633), CCL12 (p=0.0092), BAFF (p=0.0116), and VEGF (p=0.0396) compared to non-responders (FIG. 5K-P). Responders had elevated serum concentrations of IL-1 β (p=0.00135), IL-6 (p=0.0022), CCL22 (p=0.00803), GM-CSF (p=0.0108), CCL4 (p=TWEAK (p=0.02), and CCL2 (p=0.0291) compared to non-responders (FIG. 5Q-W).

Analytes that were statistically significant between responders and non-responders at both timepoints (two weeks after treatment initiation, EOS) included CCL7/MCP-3/MARC (p=2.19×10⁻⁵), CCL12/MCP-5 (p=0.000606), TWEAK/TNFSF12 (p=0.00112), BAFF/TNFSF13B (p=0.00469), IL-1 β/IL-1F2 (p=0.00507), CCL21/6Ckine (p=0.00539), VEGF (p=0.00646), IFN-γ (p=0.00817), CCL4/MIP-1β (p=0.0133), IL-6 (p=0.229), and GM-CSF (p=0.0257). The entire panel of cytokines, chemokines, and growth factors analyzed by multiplex immunoassay in murine serum from the EOS timepoint included BAFF, MCP-1, MIP-1 α, MIP-1 β, RANTES, MCP-3, Eotaxin, MCP-5, VEGFR2, MIP-3 α, CCL21, MDC, IP-10, CXCL12, GM-CSF, Granzyme B, IFN-γ, IL-1 α, IL-18, IL-2, IL-3, IL-4, IL-6, IL-7, IL-10, IL-12 p70, IL-13, IL-16, VEGF, M-CSF, Prolactin, and TWEAK.

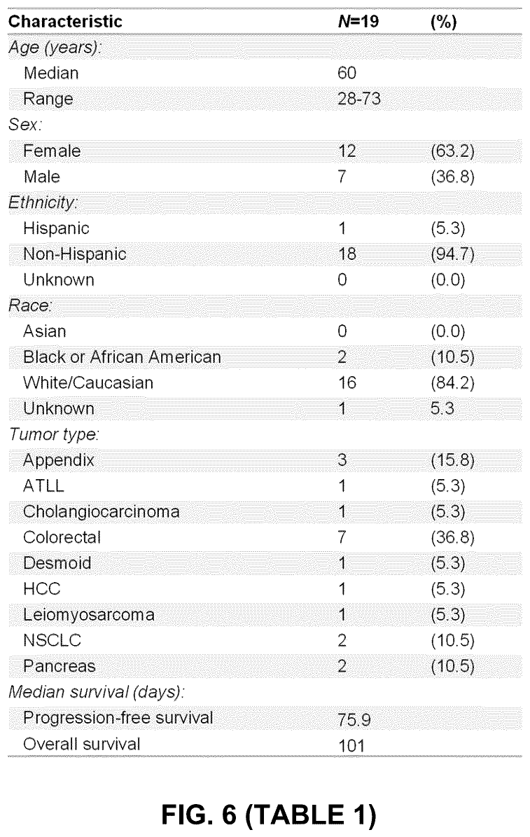

Patient plasma concentrations of cytokines from a Phase 1 clinical trial investigating elraglusib correlate with progression-free survival, overall survival, and in vivo response to therapy results. To determine the clinical relevance of the biomarkers of response identified in our murine model, the inventors next employed Luminex 200 technology to analyze plasma samples from patients with refractory solid tumors of multiple tissue origins enrolled in a Phase 1 clinical trial investigating elraglusib (NCT03678883). Patients included in this analysis represented multiple tumor types, including appendix (N=3, 15.8%), adult T-cell leukemia/lymphoma (ATLL) (N=1, cholangiocarcinoma (N=1, 5.3%), colorectal (N=7, 36.8%), desmoid (N=1, hepatocellular carcinoma (HCC) (N=1, 5.3%), leiomyosarcoma (N=1, 5.3%), non-small cell lung cancer (NSCLC) (N=2, 10.5%), and pancreas (N=2, 10.5%) cancer See FIG. 6. The median progression-free survival was 75.9 days. The median overall survival was 101 days.

TABLE 14

| | | Elraglusib | | | | | | | | |
|---|---|---|---|---|---|---|---|---|---|---|
| | | dose | Elraglusib | Number | Age at | | | | | |
| Subject | Tumor | (mg) | dose | of | enrollment | | | | PFS | OS |
| ID | type | cycle 1, cycle 2 | (mg/kg) | cycles | (years) | Sex | Race | Ethnicity | (days) | (days) |
| 05001 | Pancreas | 67 | 1 | 1 | 61 | F | White/Caucasian | Not | UN | UN |
| | | | | | | | | Hispanic/Latino | | |
| 05002 | Appendix | 47 | 1 | 1 | 59 | F | Other | Hispanic or | UN | UN |
| | | | | | | | | Latino | | |
| 05003 | Colorectal | 58 | 1 | 1 | 56 | F | White/Caucasian | Not | 26 | 26 |
| | | | | | | | | Hispanic/Latino | | |
| 05004 | Colorectal | 76 | 1 | 1 | 60 | M | White/Caucasian | Not | 28 | UN |
| | | | | | | | | Hispanic/Latino | | |
| 05005 | Colorectal | 71, 130 | 1 | 2 | 68 | M | White/Caucasian | Not | 99 | 105 |
| | | | | | | | | Hispanic/Latino | | |
| 05006 | HCC | 205 | 2 | 1 | 59 | M | White/Caucasian | Not | 234 | 234 |
| | | | | | | | | Hispanic/Latino | | |
| 05009 | Cholangiocarcinoma | 144 | 2 | 1 | 60 | M | White/Caucasian | Not | 46 | UN |
| | | | | | | | | Hispanic/Latino | | |
| 05010 | Colorectal | 182 | 2 | 1 | 51 | M | White/Caucasian | Not | UN | UN |
| | | | | | | | | Hispanic/Latino | | |
| 05011 | NSCLC | 105 | 2 | 1 | 51 | F | White/Caucasian | Not | 31 | 39 |
| | | | | | | | | Hispanic/Latino | | |
| 05013 | Colorectal | 235 | 3.3 | 1 | 70 | F | White/Caucasian | Not | 130 | UN |
| | | | | | | | | Hispanic/Latino | | |
| 05016 | Colorectal | 219 | 3.3 | 1 | 33 | F | White/Caucasian | Not | UN | UN |
| | | | | | | | | Hispanic/Latino | | |
| 05017 | Colorectal | 603 | 5 | 1 | 61 | F | Black or African | Not | 83 | UN |
| | | | | | | | American | Hispanic/Latino | | |
| 05018 | NSCLC | 305 | 5 | 1 | 73 | F | White/Caucasian | Not | 41 | UN |
| | | | | | | | | Hispanic/Latino | | |
| 05019 | Appendix | 450 | 7 | 1 | 63 | F | White/Caucasian | Not | UN | UN |
| | | | | | | | | Hispanic/Latino | | |
| 05020 | Desmoid | 741 | 7 | 1 | 28 | F | White/Caucasian | Not | UN | UN |
| | | | | | | | | Hispanic/Latino | | |
| 05024 | Appendix | 354 | 7 | 1 | 71 | F | White/Caucasian | Not | 41 | UN |
| | | | | | | | | Hispanic/Latino | | |
| 05038 | Pancreas | 766 | 9.3 | 1 | 70 | F | White/Caucasian | Not | UN | UN |
| | | | | | | | | Hispanic/Latino | | |
| 05051 | ATLL | 673 | 12.37 | 1 | 45 | M | Black or African | Not | UN | UN |
| | | | | | | | American | Hispanic/Latino | | |
| 05055 | Leiomyosarcoma | 518 | 12.37 | 1 | 67 | M | White/Caucasian | Not | UN | UN |
| | | | | | | | | Hispanic/Latino | | |

TABLE 14 description. Individual patient information for human cytokine analysis. Tumor type, elraglusib dose (mg, mg/kg), number of cycles, age at enrollment, sex, race, ethnicity, median progression free survival and median overall survival data is shown (N=19).

Baseline and twenty-four hours post-elraglusib plasma concentrations of cytokines, chemokines, and growth factors were plotted against progression-free survival and overall survival and simple linear regressions were used to calculate significance. A heatmap was then used to visualize linear regression values. The inventors found that baseline concentrations of several analytes (IL-12, CXCL11, Fas Ligand, IL-8, VEGF, IL-1 β, M-CSF, IL-2) correlated with progression-free survival (PFS). Concentrations of several analytes (IL-12, IL-1 β, IL-21, IL-8, IFN-α, IFN-γ, M-CSF, CCL4, Fas Ligand, IL-2, IL-10, CCL11, IL-15, IL-4, Granzyme B, CXCL11) twenty-four hours post dose also correlated with progression-free survival. The inventors next analyzed overall survival (OS) data and noted that baseline concentrations of several analytes (IL-8, CXCL11, CCL11, IFN-α, TNF-α, Fas Ligand, TRAIL R2, IL-1 β) correlated with overall survival.

Twenty-four hours post-dose concentrations of several analytes (IFN-α, Fas Ligand, TRAIL R2, CCL11) also correlated with overall survival. Importantly, Fas Ligand and CCL11 were the only two analytes that were statistically significant in all four comparisons (pre-dose, post-dose, overall survival, progression-free survival) and were found to be positivity correlated with improved progression-free survival and overall survival.

Many of the analytes were upregulated at eight hours and twenty-four hours after treatment as compared to baseline. When the cytokines, chemokines, and growth factors were grouped by timepoint and raw values were visualized with a heatmap, the inventors noticed several interesting trends. The inventors then grouped the tumors by grouped by primary tumor location, including appendix, adult T cell leukemia/lymphoma (ATLL), cholangiocarcinoma, colorectal, desmoid, hepatocellular carcinoma (HCC), leiomyosarcoma, non-small cell lung cancer (NSCLC), pancreatic, the patient with a desmoid tumor had elevated expression of many of the analytes included in the panel. When cytokines were grouped by elraglusib dose (1, 2, 3.3, 5, 7, 9.3, 12.37) in milligrams per kilogram, patients receiving a 7 mg/kg dose had increased expression of many of the analytes included in the panel at both the 8-hour and 24-hour post-dose timepoints. When cytokines were grouped by cytokine, chemokine, or growth factor family, TNF family molecules (BAFF, Fas Ligand, Fas, TNF-α, TRAIL R2, TRAIL R3, TRAIL, TRANCE) has a decreased expression at the 8-hour post-dose timepoint as compared to baseline and had increased over baseline levels by the 24-hour timepoint.

To compare both murine and human circulating biomarker trends, the inventors created a table to visualize major trends. EOS analyte concentrations that positively correlated with overall survival in the mouse model included IL-1 β, CCL22, CCL4, TWEAK, GM-CSF, and IL-6. Those that negatively correlated with overall survival in the mouse model included CCL21, VEGFR2, CCL12, BAFF, and VEGF. Interestingly, the inventors observed that many of these trends held when analyzing the human data. IL-1 β, CCL22, and CCL4 all were positively correlated with progression-free survival and overall survival in the human cohort. BAFF and VEGF were negatively correlated with overall survival and progression-free survival. GM-CSF and IL-6 had opposing correlations in the human cohort as compared to the murine cohort.

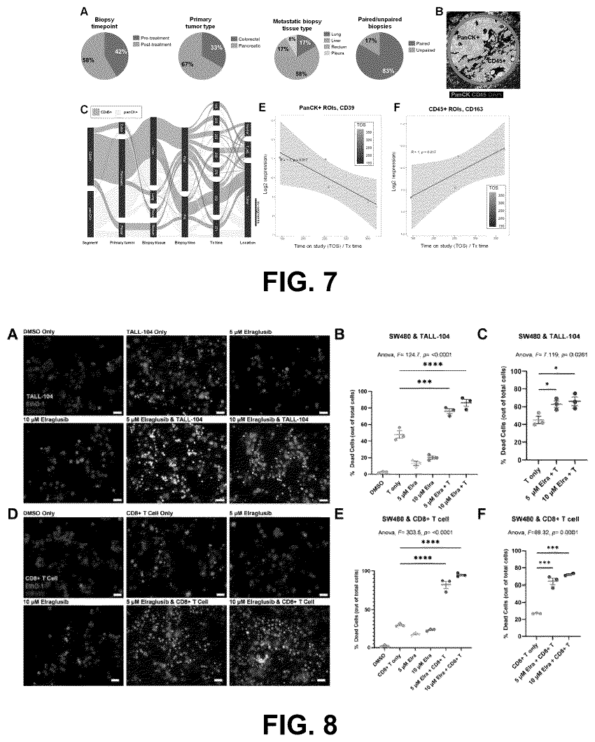

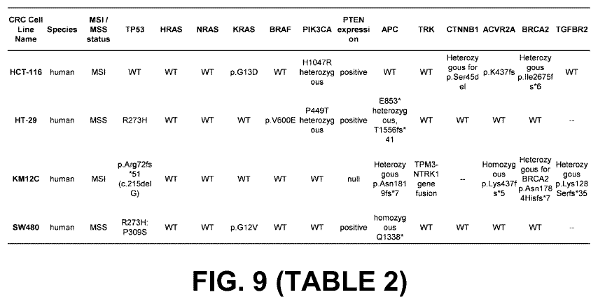

PanCK+ expression of immunosuppressive CD39 negatively correlated with time-on-treatment (Tx time) while CD45+ expression of monocyte/macrophage marker CD163 positively correlated with Tx time. The inventors used GeoMx Digital Spatial Profiling (DSP) technology to profile the expression of fifty-nine proteins in tumor biopsies (N=12) from patients treated with elraglusib (N=7). A total of 42% (N=5) of the tumor biopsies analyzed were collected near or before treatment start (pre-treatment) and 58% (N=7) of the biopsies analyzed were collected from post-treatment (average time-on-treatment (Tx time) at post-treatment biopsy: 270 days) (FIG. 7A). Primary tumor types included colorectal cancer (N=4, 33%) and pancreatic cancer (N=8, 67%), while metastatic biopsy tissue sites included lung (N=2, 17%), liver (N=7, 58%), rectum (N=2, 17%), and pleura (N=1, 8%). the inventors analyzed five paired tumor biopsies (N=slides total, 83%), and two unpaired biopsies (N=2 slides total, 17%). Half (N=6, 50%) of the tumor sections were needle biopsies and half (N=6, 50%) were tissue biopsies. All patients included in this analysis were considered responders based on the definition used in the Phase 1 trial that treatment response is equal to disease control greater than 16 weeks. Our region of interest (ROI) selection strategy focused on mixed tumor and immune cell segments within FFPE tissue. ROIs were segmented based on panCK+ and CD45+ morphology stains to compare tumor versus immune cells protein expression (FIG. 7B). the inventors utilized a PCA plot to visualize dimensionality reduction and, as expected, samples tended to cluster by tissue type (liver, lung, pleura, rectum) and further separated by segment (CD45, panCK) on PC2. The inventors used a Sankey diagram to visualize the study design, where the width of a cord in the figure represents how many segments are in common between the two annotations they connect (FIG. 7C). Approximately half of the ROIs from the entire experiment were CD45+ and the other half were panCK+. As expected, samples tended to cluster together based on patient ID, primary tumor location, biopsy timepoint, metastatic biopsy tissue site, immune cell location, or segment (CD45, panCK) type when visualized on an aggregate heatmap As the inventors were interested in the ability to predict a patient's time-on-treatment (Tx time), the inventors correlated pre-treatment protein expression levels among the responders with Tx time data and found that PanCK+ segment expression of immunosuppressive CD39 negatively correlated with Tx time (FIG. 7E), while CD45+ segment monocyte/macrophage marker CD163 expression positively correlated with Tx time (FIG. 7F). See also Borsellino et al., Blood, 110, 1225-1232 (2007).

Tumor-infiltrating immune cells have reduced inhibitory checkpoint expression and elevated expression of T cell activation markers after elraglusib treatment. When comparing all samples, CD45+ regions of post-treatment biopsies had increased protein expression of T cell activation marker OX4OL (p=0.016) and decreased protein expression of checkpoint molecules VISTA (p=2.0×10⁻²⁴), PD-L1 (p=3.2×10⁻¹³), PD-L2 (p=2.0×10⁻⁹), LAG3 (p=5.1×10⁻⁴), and PD-1 (p=5.6×10⁻⁹). CD45+ regions of post-treatment biopsies also had a decreased protein expression of myeloid/neutrophil marker CD66b (p=7.5×10⁻¹⁵), myeloid markers IDO1 (p=4.8×10⁻⁶), CD80 (p=5.4×10⁻⁶), and CD11 b (p=6.7×10⁻³), TAM/M2 macrophage marker CD68 (p=3.8×10⁻⁴), myeloid/T cell activation marker OX4OL (p=0.016), myeloid marker CD40 (p=0.020), and dendritic cell/myeloid marker CD11 c (p=0.022) as compared to pre-treatment samples (FIG. 7G). Because the inventors were interested in the differential expression of proteins based on immune cell location in relation to the tumor, the inventors annotated CD45+ ROI locations as tumor-infiltrating, tumor-adjacent, or normal tissue. When comparing tumor-infiltrating CD45+ immune cell segments in pre-treatment versus post-treatment biopsies, it was found that post-treatment tumor-infiltrating CD45+ immune cell segments had a reduced protein expression of immune checkpoints VISTA (p=1.6×10⁻¹⁴), PD-L1 (p=1.1×10⁻⁶), PD-L2 (p=7.6×10⁻⁴), and PD-1 (p=1.6×10⁻³) and elevated protein expression of T cell activation markers CTLA4 (p=3.1×10⁻⁵) and OX4OL (p=1.6×10⁻³) (FIG. 7H). Post-treatment tumor-infiltrating CD45+immune cell segments had a reduced protein expression of myeloid marker CD66b (p=1.4×10⁻¹³), antigen PTEN (p=1.5×10⁻¹¹), hematopoietic marker CD34 (p=3.3×10⁻⁹), T cell activation marker CD44 (p=3.1×10⁻⁷), antigen presentation B2M (p=2.5×10⁻⁶), immune cell activation marker HLA-DR (p=3.1×10⁻⁵), TAM/M2 macrophage marker ARG1 (p=7.6×10⁻⁵), memory T cell marker CD45R0 (p=1.1×10⁻⁴), proliferation marker Ki-67 (p=2.7×10⁻⁴), TAM/M2 macrophage marker CD68 (p=5.8×10⁻⁴), myeloid marker IDO1 (p=6.6×10⁻⁴), myeloid marker CD80 (p=2.4×10⁻³), NK cell marker CD56 (p=6.9×10⁻³), DC/myeloid marker CD11c (p=9.5×10⁻³), and T cell activation marker GITR (p=0.021) and had an elevated protein expression of immune checkpoint molecule B7-H3 (p=0.012) and Treg marker CD127 (p=0.012) as compared to pre-treatment tumor-infiltrating CD45+ immune cell segments.

Patients with a long time-on-treatment have decreased B cell and myeloid marker expression in immune cell regions and have decreased immune checkpoint expression in tumor cell regions. The inventors compared pre-treatment biopsy protein expression in CD45+ segments between patients who were undergoing treatment for a longer duration of time, called “Long Tx patients”, and patients who participated in the analysis for a shorter duration of time, called “Short Tx patients”, and observed that Long Tx patients had a reduced protein expression of B cell marker CD20 (p=0.012) and myeloid activation marker CD80 (p=0.047). Long Tx was defined as a Tx time greater than 275 days (˜39 weeks).

The inventors compared protein expression in CD45+ segments in post-treatment biopsies between Long Tx patients and Short Tx patients. the inventors observed that Long Tx patients had reduced protein expression of antigen NY-ESO-1 (p=0.021) and progesterone receptor (PR) (p=0.022). The inventors then compared pre-treatment biopsy protein expression in PanCK+ segments between Long Tx patients and Short Tx patients and observed that Long Tx patients had reduced protein expression of cytotoxic T cell marker CD8 (p=3.5×10⁻³), antigen Her2 (p=0.033), Treg marker Foxp3 (p=0.033), T cell marker CD3 (p=0.035), and B cell marker CD20 (p=0.046). Long Tx patients also had reduced immune checkpoint protein expression of LAG3 (p=0.023), PD-L2 (p=0.028), and PD-1 (p=0.046). The inventors carried out the same analysis with a focus on panCK+ segments in post-treatment biopsies. Long Tx patients had reduced protein expression of mature B cell/DC marker CD35 (p=8.5×10⁻³), antigen NY-ESO-1 (p=8.7×10⁻³), antigen Her2 (p=0.022), antigen MART1 (p=0.029), cytotoxic T cell marker CD8 (p=0.030), Treg marker Foxp3 (p=0.030), antigen PTEN (p=0.032), DC/myeloid marker CD11c (p=0.034), memory T cell marker CD45R0 (p=0.036), checkpoint PD-L1 (p=0.047), and PR (p=0.049) as compared to Short Tx patients. Several additional comparisons were made between pre-treatment and post-treatment biopsies, immune cell location in proximity to the tumor, and paired and unpaired biopsies.

Discussion. The inventors showed that the small-molecule inhibition of GSK-3 using elraglusib leads to increased natural killer and T-cell-mediated colorectal cancer cell killing in a co-culture model. elraglusib acts on tumor cells to sensitize them to immune-cell-mediated killing. This tumor cell sensitization could be resultant of drug-induced modifications in the tumor cell secretome, such as decreased VEGF expression, decreased soluble PD-L1, and increased CXCL14, as the inventors previously described by Chou, Rampanelli, Li, & Ting, Cell. Mol. Immunol., 19, 337-351 (2022) and Huntington, Zhang, Carneiro, & EI-Deiry, Cancer Res., 81, 2676 (2021).

VEGF has been shown to inhibit T cell activation. Gavalas et al., Br. J. Cancer, 107, 1869-1875 (2012). CXCL14 is a known NK cell chemoattractant. Starnes et al., Exp. Hematol., 34, 1101-1105 (2006). The soluble or shed version of PD-L1 can retain the ability to bind PD-1 and function as a decoy receptor to negatively regulate T cell function, despite being a truncated version lacking the membrane domain of the protein. Hassounah et al., Cancer Immunol. Immunother., 68, 407-420 (2019). The increase in efficacy, in combination with immune checkpoint blockade, that the inventors observed in the murine model could be due to the concomitant downregulation of sPD-L1 and upregulation of cell-surface-expressed PD-L1.

Since the inventors observed gasdermin B expression post-IFN-γ treatment in colorectal cancer cells, and because the inventors found that elraglusib treatment upregulated immune cell IFN-γ secretion, that the IFN-γ released from CD8+ T cells and NK cells should be responsible for triggering pyroptosis, which may contribute to elraglusib-mediated immunostimulation.

Another mechanism behind elraglusib-mediated immunomodulation is the suppression of inflammatory NF-κB signaling and survival pathways in the tumor cells. the inventors demonstrated that elraglusib treatment of colorectal cancer cells decreased Survivin, NF-κB p65, Bcl-2, and Mc1-1expression while increasing PD-L1 expression. This is in accordance with previous studies that have shown that GSK-3 is a positive regulator of NF-κB. Medunjanin et al., Sci. Rep., 6, 38553 (2016). Microarray data showed an increased expression of antiproliferative, proapoptotic, and NF-κB regulator genes and decreased expression of genes involved in cell cycle progression, antiapoptotic, and EMT genes in colorectal cancer cell lines. Multiplex immunoassay data showed a decreased tumor cell secretion of proteins involved in angiogenesis, EMT, and immunosuppression.

The inventors observed the opposite effect on NF-κB signaling in immune cells. Drug treatment increased NF-κB-inducing kinase (NIK) expression. NIK is the upstream kinase that regulates the activation of the non-canonical NF-κB signaling pathway and may suggest a role for non-canonical NF-κB signaling in immune cells after elraglusib treatment. The increased expression of NIK leads to an enhanced expression of chemokines and cytokines such as CCL3, TNF-α, and MCP-1, thus leading to the increased recruitment and proliferation of cytotoxic immune cells. Thu & Richmond, Cytokine Growth Factor Rev., 21, 213-226 (2010). The treatment of immune cells with elraglusib increased effector molecule secretion and increased expression of genes involved in cytotoxic granule exocytosis, cellular proliferation, and the modulation of NF-κB activity. elraglusib treatment resulted in the decreased gene expression of proapoptotic molecules and regulators of TGF signaling which may also contribute to the tumor-suppressive and anti-angiogenic effects of elraglusib that have been previously described by Park, Coveler, Cavalcante, & Saeed, Biology, 10, 610 (2021).

In a syngeneic murine colon carcinoma BALB/c murine model using MSS cell line CT-26, the inventors observed significantly improved survival in mice treated with elraglusib and anti-PD-L1 therapy. the inventors also demonstrated increased survival in mice treated with elraglusib alone as compared to the control group. The inventors also observed statistically significant improved survival in the anti-PD-1 and anti-PD-L1 alone groups as compared to the control. Responders had reduced percentages of splenic CD4+ T cells and splenic CD8+ T cells and increased percentages of CD69+ activated T cells and Foxp3+ Tregs. The increased splenic percentages of both activated and end-stage T cells in the responder groups could be indicative of an anti-tumor immune response that was mounted earlier in the course of treatment.

Compared to non-responders, responders also had more CD3+ and CD4+ tumor-infiltrating lymphocytes. Further studies could evaluate the contribution of CD4+ versus CD8+ tumor-infiltrating T cells to the observed response to elraglusib and anti-PD-L1 therapy, especially considering the recent interest in the contribution of CD4+ helper T cells to anti-tumor immunity. Tay, Richardson, & Toh, Cancer Gene Ther., 28, 5-17 (2021). The inventors did not observe many significant differences in splenic NK cell subpopulations in either the tumor or the spleen, although perhaps the timepoint the inventors chose to analyze was not representative of NK cell subpopulation changes that may have occurred earlier or later in the course of treatment.

Murine responders had reduced serum concentrations of BAFF, CCL7, CCL12, VEGF, VEGFR2, and CCL21. BAFF is a cytokine that belongs to the TNF ligand superfamily, which may promote tumorigenesis indirectly through the induction of inflammation in the tumor microenvironment (TME) and directly through the induction of EMT. Rihacek et al., BioMed. Res. Int., 792187 (2015). CCL7 enhances both cancer progression and metastasis via EMT, including in CRC cells. Lee et al., Oncotarget, 7, 36842-36853 (2016). Others have demonstrated that CXCR4 plays a critical role in the promotion of the progression of inflammatory CRC. Yu et al., J. Exp. Clin. Cancer Res., 38, 32 (2019). The expression of VEGF-1 in CRC is associated with disease localization, stage, and long-term survival. Bendardaf et al., Anticancer Res., 28, 3865-3870 (2008). The inventors previously observed the suppression of VEGF in a panel of CRC cell lines post-elraglusib treatment and saw a similar suppression of VEGF in the murine responders. the inventors noted a decrease in VEGFR2 in murine responders, a protein that is highly expressed in CRC and promotes angiogenesis. Zhong et al., Int. J. Biol. Sci., 16, 272-283 (2020). CCL21 has been shown to play a role in colon cancer metastasis. Li, Sun, Tao, & Wang, Dig. Liver Dis., 43, 40-47 (2011).

Responders had elevated serum concentrations of CCL4, TWEAK, GM-CSF, CCL22, and IL-12p70 as compared to non-responders. Others have demonstrated that CCL4 is an important chemokine in the tumor microenvironment, determining response to immune checkpoint blockade, and that a lack of CCL4 can lead to the absence of CD103+ dendritic cells (DCs). Williford et al., Sci. Adv., 5, eaay1357 (2019). DCs are an important cell population influencing the response to immune checkpoint blockade, and although the inventors did not monitor their levels in this analysis, it is conceivable that they played a role in influencing response to therapy. For this reason, further studies could monitor dendritic cell populations during the course of therapy. TWEAK is commonly expressed by peripheral blood monocytes and upregulates its expression after exposure to IFN-γ. Nakayama et al., J. Exp. Med., 192,1373-1380 (2000). TWEAK has also been shown to promote the nuclear translocation of both classical and alternative NF-κB pathway subunits. Saitoh et al., J. Biol. Chem., 278, 36005-36012 (2003). GM-CSF is a well-known immunomodulatory factor that has immunostimulatory functions but is also predictive of poor prognosis in CRC. Taghipour Fard Ardekani et al., J. Mol. Cell. Med., 3,27-34 (2014). The inventors observed increased levels of IL-12p70 in murine responders. IL-12 is a potent, pro-inflammatory cytokine that has been shown to increase the activation and cytotoxicity of both T and NK cells as well as to inhibit immunosuppressive cells such as TAMs and myeloid-derived suppressor cells (MDSCs). Watkins, Egilmez, Suttles, & Stout, J. Immunol., 178, 1357-1362 (2007); 51. Steding et al., 133,221-238 (2011).

The inventors demonstrated that GSK-3 inhibitors such as elraglusib can be a combination strategy to increase the efficacy of immune checkpoint blockade in patients with MSS CRC. The elraglusib-mediated increase in tumor surface-cell-expressed PD-L1 should make this a good small molecule to combine with anti-PD-L1 therapies.

The cytokine analysis of plasma samples from patients with refractory solid tumors of multiple tissue origins enrolled in a Phase 1 clinical trial investigating elraglusib (NCT03678883) revealed that elevated baseline plasma levels of proteins such as IL-1 β and reduced levels of proteins such as VEGF correlated with improved progression-free survival and overall survival. Progression-free survival was also found to be positively correlated with elevated plasma levels of immunostimulatory analytes such as Granzyme B, IFN-γ, and IL-2 at twenty-four hours after elraglusib treatment. Several of these secreted proteins correlated with results from the in vivo analysis, where the expression of proteins such as IL-1 β, CCL22, CCL4, and TWEAK was positively correlated with improved response to therapy while the expression of proteins such as BAFF and VEGF negatively correlated with response to therapy. Huntington et al., Abstract 4166: Small-molecule inhibition of glycogen synthase kinase-3 (GSK-3) increases the efficacy of anti-PD-L1 therapy in a murine model of microsatellite stable colorectal cancer (CRC); Therapeutic response correlates with T cell ratios and serum cytokine profiles in mice. Cancer Res., 82, 4166 (2022). These results introduce consider biomarkers for correlations with response to therapy, which could provide significant clinical utility.

DSP analysis of paired FFPE tumor biopsies from patients with CRC or pancreatic cancer before and after treatment revealed that CD39 expression in PanCK+ segments was negatively correlated with duration of treatment, while CD163 expression in CD45+ segments was positively correlated with duration of treatment and potential therapeutic benefit. CD39 can inhibit costimulatory signaling, increase immunosuppression during T cell priming, and its expression is associated with TAMs, Tregs, and inhibited cytotoxic immune cell function. Allard, Allard, & Stagg, J. Immunother. Cancer, 8, e000186 (2020). CD39 has been shown to suppress pyroptosis and impair immunogenic cell death. CD39 expression on endothelial cells regulates the migration of immune cells and promotes angiogenesis. Allard, Allard, & Stagg, J. Immunother. Cancer, 8, e000186 (2020). CD163 is a marker of cells from the monocyte/macrophage lineage. Immune cell segments showed differential protein expression based on the proximity to the tumor where tumor-infiltrating immune cells had decreased expression of immune checkpoints (PD-L1, Tim-3, PD-1) and Treg markers (CD25, CD127) as compared to tumor-adjacent immune cells, regardless of timepoint. The downregulation of immune checkpoint proteins PD-1, TIGIT, and LAG-3 by elraglusib has been previously described in melanoma models. Shaw, Cavalcante, Giles, & Taylor, J. Hematol. Oncol., 15, 134 (2022).

Interestingly, the inventors observed a downregulation of PD-L1 expression in PanCK+ segments at the post-treatment biopsy as compared to the pre-treatment biopsies. This was unexpected as we, and others, have shown that GSK-3 inhibition upregulates tumor cell PD-L1 expression. Li et al., Nature Commun., 7, 12632 (2016); Huntington et al., bioRxiv (2023). The observed decrease in PanCK+PD-L1 expression may be a result of the late timepoint of the post-treatment biopsies, as the average time-on-analysis at the post-treatment timepoint was 270 days.

When the inventors analyzed differential protein expression between Long Tx patients and Short Tx patients, the inventors found that Long Tx patients had a reduced post-treatment expression of mature B cell/DC marker CD35, antigen NY-ESO-1, antigen Her2, antigen MART1, cytotoxic T cell marker CD8, Treg marker Foxp3, antigen PTEN, dendritic cell/myeloid marker CD11c, memory T cell marker CD45RO, checkpoint PD-L1, and PR in PanCK+ segments as compared to Short Tx patients, which introduces several potential biomarkers of response to GSK-3 therapy, which should be validated in further studies. When the inventors compared post-treatment protein expression in tumor-infiltrating CD45+ immune cell segments in Long Tx patients and Short Tx patients, the inventors found that Long Tx patients had a decreased expression of antigens NY-ESO-1, PTEN, and PR as compared to Short Tx patients. Interestingly, these three antigens (NY-ESO-1, PTEN, and PR) had decreased expression in Long Tx patients post-treatment, regardless of tumor or immune cell region.

This EXAMPLE demonstrates that the small-molecule inhibition of GSK-3 using elraglusib may be a potential means to increase the efficacy of immune checkpoint blockade and improve response in patients with MSS CRC and other tumor types. This analysis is the first digital spatial analysis of tumor biopsies from patients treated with elraglusib and very few oncology drugs have been evaluated using GeoMx technology to date. The circulating biomarkers of response to GSK-3 inhibition identified using the cytokine profiling data could provide significant clinical utility and the spatial proteomics data provide us with insights into the immunomodulatory mechanisms of GSK-3 inhibition.

### LIST OF EMBODIMENTS

Specific compositions and methods of the invention have been described. The detailed description in this specification is illustrative and not restrictive or exhaustive. The detailed description does not intend to limit the disclosure to the precise form described. Other equivalents and modifications besides those already described are possible without departing from the inventive concepts described in this specification, as persons having ordinary skill in the biomedical art will recognize. When the specification or claims recite method steps or functions in an order, alternative embodiments may perform the functions in a different order or concurrently. The inventive subject matter should not be restricted except in the spirit of the disclosure.

All terms should be interpreted in the broadest possible manner consistent with the context when interpreting the disclosure. Unless otherwise defined, the technical and scientific terms used in this specification have the same meaning as commonly understood by persons having ordinary skill in the biomedical art. This invention is not limited to the particular methods, protocols, and reagents described in this specification and can vary in practice. The invention is defined only by the claims.

When a range of values is provided, each intervening value, to the tenth of the unit of the lower limit, unless the context dictates otherwise, between the upper and lower limit of that range and any other stated or intervening value in that range of values.

### REFERENCES

Persons having ordinary skill in the biomedical art can rely on these patents, patent applications, scientific books, and scientific publications for enabling methods:

### Patent Citations.

PCT Pat. Publ. WO2019/236703 (Actuate Therapeutics Inc.)
PCT Pat. Publ. WO2029/123813 (Actuate Therapeutics Inc.)
PCT Pat. Publ. WO2022/109057 (Brown University)

### Non Patent Citations.

Allard, Allard, & Stagg, On the mechanism of anti-CD39 immune checkpoint therapy. J. Immunother. Cancer, 8, e000186 (2020).
Araki et al., Clinical significance of serum soluble IL-2R levels in patients with adult T cell leukaemia (ATL) and HTLV-1 carriers. Clin. Exp. Immunol., 119, 259-263 (2000).
Augello et al., The role of GSK-3 in cancer immunotherapy: GSK-3 Inhibitors as a new frontier in cancer treatment. Cells, 9, 1427 (2020).
Bendardaf et al., VEGF-1 expression in colorectal cancer is associated with disease localization, stage, and long-term disease-specific survival. Anticancer Res., 28, 3865-3870 (2008).
Bernardo et al., An experimental model of anti-PD-1 resistance exhibits activation of TGFβ and Notch pathways and is sensitive to local mRNA immunotherapy. Oncoimmunology, 10, 1881268 (2021).
Borelli et al., Immune-checkpoint inhibitors (IC's) in metastatic colorectal cancer (mCRC) patients beyond microsatellite instability. Cancers, 14, 4974 (2022).
Borsellino et al., Expression of ectonucleotidase CD39 by Foxp3+ Treg cells: Hydrolysis of extracellular ATP and immune suppression. Blood, 110, 1225-1232 (2007).
Brandt et al., The B7 family member B7-H6 is a tumor cell ligand for the activating natural killer cell receptor NKp30 in humans. J. Exp. Med., 206, 1495-1503 (2009).
Campeau et al., A versatile viral system for expression and depletion of proteins in mammalian cells. PLoS ONE, 4, e6529 (2009).
Carneiro et al., Phase I study of 9-ing-41, a small molecule selective glycogen synthase kinase-3 beta (GSK-3β) inhibitor, as a single agent and combined with chemotherapy, in patients with refractory tumors. J. Clin. Oncol., 38, 3507 (2020).
Castro et al., Interferon-gamma at the crossroads of tumor immune surveillance or evasion. Front. Immunol., 9, 847 (2018).
Chou, Rampanelli, Li, & Ting, Impact of intracellular innate immune receptors on immunometabolism. Cell. Mol. Immunol., 19,337-351 (2022).
Cohen & Frame, The renaissance of GSK3. Nature Rev. Mol. Cell Biol., 2, 769-776 (2001).
Ding et al. Glycogen synthase kinase-3 inhibition sensitizes pancreatic cancer cells to chemotherapy by abrogating the TopBP1/ATR-mediated DNA damage response. Clin. Cancer Res., 25,6452-6462 (2019).
EI-Deiry et al., Abstract e15138. GSK-3 inhibitor elraglusib (9-ING-41) to enhance tumor-infiltrating immune cell activation in tumor biopsies and synergize with anti-PD-L1 in a murine model of colorectal cancer. Journal of Clinical Oncology (May 31, 2023) (2023 ASCO Annual Meeting).
Fischer, Census and evaluation of p53 target genes. Oncogene, 36, 3943-3956 (2017).
Force & Woodgett, Unique and overlapping functions of GSK-3 isoforms in cell differentiation and proliferation and cardiovascular development. J. Biol. Chem., 284,9643-9647 (2009).
Fuchs, Spiegelman, & Suresh Kumar, The many faces of β-TrCP E3 ubiquitin ligases: Reflections in the magic mirror of cancer. Oncogene 2004, 23, 2028-2036.
Gavalas et al., VEGF directly suppresses activation of T cells from ascites secondary to ovarian cancer via VEGF receptor type 2. Br. J. Cancer, 107,1869-1875 (2012).
Gupta, Sinha, & Paul, The impact of microsatellite stability status in colorectal cancer. Curr. Probl. Cancer, 42,548-559 (2018).
Hassounah et al., Identification and characterization of an alternative cancer-derived PD-L1 splice variant. Cancer Immunol. Immunother. 2019, 68, 407-420.
Honey, CCL3 and CCL4 actively recruit CD8+ T cells. Nature Rev. Immunol., 6, 427 (2006).
Huntington et al., Abstract 4166: Small-molecule inhibition of glycogen synthase kinase-3 (GSK-3) increases the efficacy of anti-PD-L1 therapy in a murine model of microsatellite stable colorectal cancer (CRC); Therapeutic response correlates with T cell ratios and serum cytokine profiles in mice. Cancer Research, 82 (12 Supplement), 4166 (Jun. 15, 2022).
Huntington et al., Abstract 5636: Multiplex digital spatial profiling (DSP) of proteins in the tumor microenvironment in response to GSK-3 inhibition by 9-ING-41 (elraglusib) correlates with immunostimulatory effects observed in vivo. In: Proceedings of the American Association for Cancer Research Annual Meeting 2022; 2022 Apr. 8-13. Philadelphia (PA): AACR; Cancer Research, 83 (7 Supplement): 5636 (Apr. 4, 2023).
Huntington et al., Chemotherapy-induced cytokines and prognostic gene signatures vary across breast and colorectal cancer. Am J Cancer Res., 11(12), 6086-6106 (2021).
Huntington et al., GSK-3 inhibitor elraglusib enhances tumor-infiltrating immune cell activation in tumor biopsies and synergizes with anti-PD-L1 in a murine model of colorectal cancer, International Journal of Medical Sciences, 24(13), 10870 (Jun. 29, 2023); bioRxiv (2023).
Huntington, Louie, Zhou & EI-Deiry, A high-throughput customized cytokinome screen of colon cancer cell responses to small-molecule oncology drugs. Oncotarget, 12, 1980-1991 (2021).
Huntington, Zhang, Carneiro, & EI-Deiry, Abstract 2676: GSK3β inhibition by small molecule 9-ING-41 decreases VEGF and other cytokines, and boosts NK and T cell-mediated killing of colorectal tumor cells. Cancer Res., 81, 2676 (2021); TB06 Tumor Microenvironment (Nov. 18, 2020).
Kaler, Augenlicht, & Klampfer, Activating mutations in β-Catenin in colon cancer cells alter their interaction with macrophages; The role of Snail. PLoS ONE, 7, e45462 (2012).
Kim et al., Death induction by recombinant native TRAIL and its prevention by a caspase 9 inhibitor in primary human esophageal epithelial cells. J. Biol. Chem., 279, 40044-40052 (2004).
Lee et al., Crosstalk between CCL7 and CCR3 promotes metastasis of colon cancer cells via ERK-JNK signaling pathways. Oncotarget, 7, 36842-36853 (2016).
Li et al., Glycosylation and stabilization of programmed death ligand-1 suppresses T-cell activity. Nature Commun., 7, 12632 (2016).
Li, Sun, Tao, & Wang, The CCL21/CCR7 pathway plays a key role in human colon cancer metastasis through regulation of matrix metalloproteinase-9. Dig. Liver Dis., 43, 40-47 (2011).
Lu, Guo, & Zhang, Pyroptosis in cancer: Friend or foe? Cancers, 13, 3620 (2021).
Mancinelli et al., Multifaceted Roles of GSK-3 in Cancer and Autophagy-Related Diseases. Oxid. Med. Cell Longev. 2017, 2017, 4629495.
Maraskovsky, Chen, & Shortman, IL-2 and IFN-gamma are two necessary lymphokines in the development of cytolytic T cells. J. Immunol., 143, 1210-1214 (1989).
McClanahan et al., PD-L1 checkpoint blockade prevents immune dysfunction and leukemia development in a mouse model of chronic lymphocytic leukemia. Blood, 126,203-211 (2015).
Medunjanin et al., GSK-36 controls NF-kappaB activity via IKKγ/NEMO. Sci. Rep., 6,38553 (2016).
Mezzadra et al., Identification of CMTM6 and CMTM4 as PD-L1 protein regulators. Nature, 549, 106-110 (2017).
Middha et al., Majority of B2M-mutant and -deficient colorectal carcinomas achieve clinical benefit from immune checkpoint inhibitor therapy and are microsatellite instability-high. JCO Precis. Oncol., 3, 1-14 (2019).
Nakayama et al., Involvement of TWEAK in interferon gamma-stimulated monocyte cytotoxicity. J. Exp. Med., 192, 1373-1380 (2000).
Quesada-Romero et al. Insights into the Interactions between maleimide derivates and GSK3b combining molecular docking and QSAR,” PLoS One, 9(7), 1-11 (Jul. 10, 2014).
Pal et al., Inhibition of GSK-3 induces differentiation and impaired glucose metabolism in renal cancer. Molecular Cancer Therapeutics, 13(2), 285-296 (Feb. 1, 2014).
Park, Coveler, Cavalcante, & Saeed, GSK-3β in Pancreatic Cancer: Spotlight on 9-ING-41, Its Therapeutic Potential and Immune Modulatory Properties. Biology, 10, 610 (2021).
Proietti, Carneiro-Proietti, Catalan-Soares, & Murphy, Global epidemiology of HTLV-I infection and associated diseases. Oncogene, 24, 6058-6068 (2005).
Rihacek et al., B-Cell activating factor as a cancer biomarker and its implications in cancer-related cachexia. BioMed. Res. Int., 792187 (2015).
Rowan et al., APC mutations in sporadic colorectal tumors: A mutational “hotspot” and interdependence of the “two hits”. Proc. Natl. Acad. Sci. USA, 97, 3352 (2000).
Rudd, Chanthong, & Taylor, Small molecule inhibition of GSK-3 specifically inhibits the transcription of inhibitory co-receptor LAG-3 for enhanced anti-tumor immunity. Cell Rep., 30, 2075-2082.e2074 (2020).
Sahin et al., Glycogen synthase kinase-3 beta inhibitors as novel cancer treatments and modulators of antitumor immune responses,” Cancer Biology & Therapy (Apr. 12, 2019).
Saitoh et al., TWEAK induces NF-kappaB2 p100 processing and long lasting NF-kappaB activation. J. Biol. Chem., 278,36005-36012 (2003).
Shaw, Cavalcante, Giles, & Taylor, Elraglusib (9-ING-41), a selective small-molecule inhibitor of glycogen synthase kinase-3 beta, reduces expression of immune checkpoint molecules PD-1, TIGIT and LAG-3 and enhances CD8(+) T cell cytolytic killing of melanoma cells. J. Hematol. Oncol., 15, 134 (2022).
Sideras et al., Prognostic value of intra-tumoral CD8+/FoxP3+ lymphocyte ratio in patients with resected colorectal cancer liver metastasis. J. Surg. Oncol., 118, 68-76 (2018).
Starnes et al., The chemokine CXCL14 (BRAK) stimulates activated NK cell migration: Implications for the downregulation of CXCL14 in malignancy. Exp. Hematol., 34, 1101-1105 (2006).
Steding et al., The role of interleukin-12 on modulating myeloid-derived suppressor cells, increasing overall survival and reducing metastasis. Immunology, 133, 221-238 (2011).
Taghipour Fard Ardekani et al., Evaluation of pre-treatment serum levels of IL-7 and GM-CSF in colorectal cancer patients. Int. J. Mol. Cell. Med., 3, 27-34 (2014).
Tang et al., Effects of Glycogen Synthase Kinase-3β Inhibitor TWS119 on Proliferation and Cytokine Production of TILs From Human Lung Cancer. J. Immunother., 41, 319-328 (2018).
Tay, Richardson, & Toh, Revisiting the role of CD4+ T cells in cancer immunotherapy—New insights into old paradigms. Cancer Gene Ther., 28, 5-17 (2021).
Taylor & Rudd, Glycogen synthase kinase 3 (GSK-3) controls T-cell motility and interactions with antigen presenting cells. BMC Res. Notes, 13, 163 (2020).
Taylor et al., Glycogen synthase kinase 3 inactivation drives T-bet-mediated downregulation of co-receptor PD-1 to enhance CD8(+) cytolytic T cell responses. Immunity 2016, 44, 274-286.
Thu & Richmond, NF-κB inducing kinase: A key regulator in the immune system and in cancer. Cytokine Growth Factor Rev., 21, 213-226 (2010).
Tsai et al., Glycogen synthase kinase-36 facilitates cytokine production in 12-O-tetradecanoylphorbol-13-acetate/ionomycin-activated human CD4(+) T lymphocytes. Cells, 9, 1424 (2020).
Ugolkov et al., 9-ING-41, a small-molecule glycogen synthase kinase-3 inhibitor, is active in neuroblastoma. Anticancer Drugs, 29, 717-724 (2018).
Watkins, Egilmez, Suttles, & Stout, IL-12 rapidly alters the functional profile of tumor-associated and tumor-infiltrating macrophages in vitro and in vivo. J. Immunol., 178, 1357-1362 (2007).
Weiskittel et al., Plasma cytokine profiles and survival outcomes in the 1801 phase 1/2 clinical trial of 9-ING-41 (elraglusib) in patients with advanced cancer. e14555, 2023 by American Society of Clinical Oncology.
Williford et al., Recruitment of CD103(+) dendritic cells via tumor-targeted chemokine delivery enhances efficacy of checkpoint inhibitor immunotherapy. Sci. Adv., 5, eaay 1357 (2019).
Yu et al., CXCL12/CXCR4 promotes inflammation-driven colorectal cancer progression through activation of RhoA signaling by sponging miR-133a-3p. J. Exp. Clin. Cancer Res., 38, 32 (2019).
Zeng et al., Effect of IPP5, a novel inhibitor of PP1, on apoptosis and the

underlying mechanisms involved. Biotechnol. Appl. Biochem., 54, 231-238 (2009).
Zhang et al. Differential activity of GSK-3 isoforms regulates NF-κB and TRAIL- or TNFα induced apoptosis in pancreatic cancer cells. Cell Death Dis., 5, e1142 (2014).
Zhang et al., NEK2 inhibition triggers anti-pancreatic cancer immunity by targeting PD-L1. Nature Commun., 12, 4536 (2021).
Zhong et al., TIPE regulates VEGFR2 expression and promotes angiogenesis in colorectal cancer. Int. J. Biol. Sci., 16, 272-283 (2020).
Zhou et al., Granzyme A from cytotoxic lymphocytes cleaves GSDMB to trigger pyroptosis in target cells. Science, 368, 2-7 (2020).

### Textbooks and Technical References.

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB), (2014). Frederick M. Ausubel (ed.), John Wiley and Sons (ISBN 047150338X, 9780471503385).
Current Protocols in Protein Science (CPPS), (2005). John E. Coligan (ed.), John Wiley and Sons, Inc.
Immunology (2006). Werner Luttmann, published by Elsevier.
Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
The Encyclopedia of Molecular Cell Biology and Molecular Medicine, Robert S. Porter et al., (eds.), published by Blackwell Science Ltd., 1999-2012 (ISBN 9783527600908).
The Merck Manual of Diagnosis and Therapy, 19^{th} edition (Merck Sharp & Dohme Corp., 2018).
Pharmaceutical Sciences 23^{rd} edition (Elsevier, 2020).

All patents and publications cited throughout this specification are incorporated by reference to disclose and describe the materials and methods that might be used with the technologies described in this specification. The publications discussed are provided only for their disclosure before the filing date. They should not be construed as an admission that the inventors may not antedate such disclosure under prior invention or for any other reason. If there is an apparent discrepancy between a prior patent or publication and the description provided in this specification, the specification (including any definitions) and claims shall control. The statements about the date or contents of these documents are based on the information available to the applicants. These statements constitute no admission to the correctness of the dates or contents of these documents. The publication dates provided in this specification may differ from the actual publication dates. If there is an apparent discrepancy between a publication date provided in this specification and the actual publication date supplied by the publisher, the actual publication date shall control.

We claim:

1. A method of treating cancer, comprising the steps of:
(a) identifying a subject with likelihood of having a cancer, wherein the subject has one or more of the symptoms of cancer
(b) obtaining a sample from the subject
(c) analyzing biomarkers
(d) administering an anti-cancer treatment;
wherein the biomarkers are selected from the group consisting of:
elevated pre-pharmacokinetic (PK) plasma concentrations of IL-12, Fas Ligand, IL-8, M-CSF, IL-2, IL-15, CCL7, and CCLII correlated with improved progression-free survival (PFS) in days;
decreased plasma concentrations of CXCLII and VEGF correlated with improved progression-free survival;
at the 8-hour post-PK timepoint, increased IL-8 concentrations correlated with improved progression-free survival;
at the 24-hour post-PK timepoint, increased levels of IL-12, IL-1 beta, IL-21, IL-8, IFN-alpha, IFN-gamma, M-CSF, CCL4, Fas Ligand, IL-2, IL-10, CCLII, IL-15, IL-4, and Granzyme B were correlated with improved progression-free survival;
reduced CXCLII plasma concentrations correlated with worsened progression-free survival;
elevated IL-8, CCLII, IFN-alpha, Fas Ligand, TRAIL R2, and IL-1 beta were correlated with improved overall survival;
decreased levels of CXCLII and TNF-alpha were correlated with improved overall survival.
at the 8-hour post-PK timepoint CCL22 and IL-8 levels were positively correlated with overall survival;
at the 24-hour post-PK timepoint IFN-alpha, Fas Ligand, TRAIL R2, and CCLII levels were positively correlated with overall survival;
complete and partial responders, regardless of treatment group, are more likely to have lower serum concentrations of BAFF, CCL7, CCL12, VEGF, VEGFR2, and CCL21 compared to non-responders; and
complete and partial responders have higher serum concentrations of CCL4, TWEAK, GM-CSF, CCL22, and IL-12p70 compared to non-responders.

2. The method of claim 1, wherein the biomarkers are circulating biomarkers.

3. The method of claim 1, wherein the cancer is microsatellite stable (MSS) colorectal cancer.

4. The method of claim 1, wherein the treatment is a combination of immune checkpoint blockade with small molecules in oncology.

5. The method of claim 1, wherein the treatment is a use of anti-PD-L1 treatment.

6. The method of claim 1, wherein key biomarkers of response are evaluated at baseline in treatment-naïve patients and monitored longitudinally and assist in the evaluation of tumor response to treatment and guide therapeutic decisions. These biomarkers are response markers to immune checkpoint blockade and GSK-3 inhibition.
